(19) United States
(12) Patent Application Publication
(10) Pub. No.: US 2026/0183414 A1
(43) Pub. Date: Jul. 2, 2026

(54) Methods of Treating Cancer Using Anti-HER2 Antibody-Drug Conjugates and Anti-PD-1 Antibodies

(71) Applicant: RemeGen Co., Ltd., Yantai (CN)

(72) Inventors: Jianmin Fang, Yantai (CN); Xiaohong Su, Yantai (CN)

(73) Assignee: RemeGen Co., Ltd., Yantai (CN)

(21) Appl. No.: 19/564,737

(22) Filed: Mar. 12, 2026

Related U.S. Application Data
(63) Continuation of application No. PCT/CN2024/110142, filed on Aug. 6, 2024.

(30) Foreign Application Priority Data
Sep. 14, 2023 (WO) PCT/CN23/118900

(51) Int. Cl. A61K 47/68; A61K 39/00; A61K 39/395; A61P 35/04

(52) U.S. Cl. CPC A61K 47/6851; A61K 39/3955; A61K 47/68031; A61P 35/04; A61K 2039/505

(57) ABSTRACT

Provided herein are methods for treating or preventing progression of cancer in an individual, comprising administering to the individual an effective amount of an anti-PD-1 antibody, e.g., toripalimab, and an anti-human epidermal growth factor receptor 2 (HER2) antibody-drug conjugate that comprises an anti-HER2 antibody and a cytotoxic molecule. Compositions, uses, and kits related thereto are also provided.

### CROSS-REFERENCE TO RELATED APPLICATIONS

This application is a continuation application of International Application No. PCT/CN2024/110142, filed Aug. 6, 2024, which claims the benefit of priority of International Application No. PCT/CN2023/118900, filed on Sep. 14, 2023, the contents of each of which are incorporated herein by reference in their entirety.

### REFERENCE TO AN ELECTRONIC SEQUENCE LISTING

This application contains an electronic sequence listing, which has been submitted electronically in.xml format. The contents of the electronic sequence listing (file name: 761682010741seqlist.xml; size: 22,262 bytes; and date of creation: Aug. 1, 2024) are herein incorporated by reference in their entirety.

### FIELD

The present disclosure relates to methods for treating or preventing progression of cancer in an individual, as well as compositions, uses, and kits related thereto. In some embodiments, the methods comprise administering to the individual an effective amount of an anti-PD-1 antibody, e.g., toripalimab, and an anti-human epidermal growth factor receptor 2 (HER2) antibody-drug conjugate that comprises an anti-HER2 antibody and a cytotoxic molecule.

### BACKGROUND

ErbB2, also known as HER2/neu, is a member of a family of tyrosine kinases that regulate cell growth and survival (Lengyel, C. G. et al. (2021) Gastrointest Disord 3(1): 1-22). Overexpression and/or amplification of HER2 is seen in many malignancies including breast, gastric, ovarian, pancreatic, colorectal, and endometrial cancer (Neve, R. M. et al. (2001) Ann Oncol 12 (Suppl1): S9-S13; Menard, S. et al. (2003) Oncogene 22 (42): 6570-6578; Moasser, M. M. (2007) Oncogene 26 (45): 6469-6487; Iqbal, N. et al. (2014) Mol Biol Int 2014:852748). Tumors that overexpress HER2 are thought to be more aggressive and associated with poorer overall survival (OS) compared to HER2-negative cancers. Cancers characterized by overexpression of HER2 (e.g., HER2-positive cancers) are often correlated with poor prognosis and/or are resistant to many standard therapies. The introduction of HER2-targeted therapy using either antibody-based therapies has led to significant and ongoing improvements in disease-free survival (DFS), progression-free survival (PFS), and OS in both the neoadjuvant/adjuvant and metastatic settings (Slamon, D. J. et al. (2001) N Engl J Med 344 (11): 783-792; Geyer, C. E. et al. (2006) N Engl J Med 355 (26): 2733-2743; Baselga 2012; Verma 2012). HER2 is an effective therapeutic target in multiple solid tumors with anti-HER2 biologic agents, antibody-drug conjugates (ADC), and small molecule drugs approved for patients with HER2 overexpressing/amplified (hereafter HER2+) breast and gastric cancers. As such, a handful of therapies targeting HER2 have been approved for treatment of HER2-positive cancer, including antibodies such as trastuzumab and pertuzumab, antibody-drug conjugates (ADCs) such as trastuzumab maytansine (T-DM1).

Among the multiplicity of anticancer agents that have been developed in the last few decades, antibody-drug conjugates (ADCs) stand out in their ability to selectively deliver highly potent cytotoxic agents to target cancer cells, thus improving efficacy and limiting toxicities. ADCs are composed of three main components: an antibody, a linker, and a payload. Their mechanism of action can be summarized as follows: upon the binding of the antibody (Ab) to the antigen expressed on the surface of target cell, the ADC is internalized and processed to release the payload, which exerts its effects leading to cell death. Simultaneously with the development of this drug class, a paradigm shift occurred in a wide variety of cancer types with the evaluation and implementation of immunotherapy drugs becoming increasingly prominent. Immune checkpoint inhibitors (ICIs) targeting the PD-1/PD-L1 axis have shown unprecedented clinical activity in multiple cancer types. These agents, reversing the tumor-mediated immune-cell suppression, have the potential to achieve durable responses. Unfortunately, only a limited fraction of patients experiences long-term benefit when treated with single-agent immunotherapies due to development of resistance to ICIs and/or limited clinical utility of ICIs. Combination treatments with other immunotherapies or different treatment modalities are needed to overcome these issues. Evidence in phase III clinical trials already supports combinations of immunotherapy with standard-of-care chemotherapy for a number of cancers. However, concerns related to possible increased toxicity or impaired efficacy, arise with combination treatments. In this regard, ADCs have the potential to induce less off-target toxicities and appear as a promising companion for combination strategies with ICIs. Available clinical evidence for combination treatment with ADCs and immunotherapy is reviewed in Nicolò, E. et al. (2022), Cancer Treat Rev. 106:102395.

Gastric and gastroesophageal junction (GEJ) cancer is one of the most common malignancies worldwide. In unresectable or metastatic disease, the prognosis is poor and is generally less than a year. Standard front-line chemotherapy includes two- or three-drug regimens with the addition of trastuzumab in HER2-positive disease. With an increased understanding of the biology of cancer over the past few decades, targeted therapies have made their way into the treatment paradigm of many cancers. They have been examined in the first- and second-line settings in the treatment of GEJ cancer, but such studies have yielded viable treatment options. One success is ramucirumab either as monotherapy or in combination with paclitaxel, which is the preferred choice in second-line therapy. While immunotherapy has been considered a breakthrough in oncology over the past decade, the response rates in gastric and GEJ cancers have been relatively low compared to other cancers, resulting in its limited approval and mostly reserved for second-line therapy or beyond. Pembrolizumab, a monoclonal antibody targeting programmed cell death protein-1 (PD-1), is the most studied ICI in GEJ and gastric cancers. See, Hsu, A. et al. (2020) Ann Transl Med 8 (17): 1109. Over the last decades, targeted therapy and immunotherapy emerge as revolutionary breakthroughs in cancer treatment. The success of the ToGA trial shed light on HER2-targeted therapy in gastric cancer. (Deng, T. et al. (2023) Front Oncol 13:1166040) In 2019, KEYNOTE-062 examined the use of pembrolizumab monotherapy versus pembrolizumab with cisplatin and fluorouracil/capecitabine versus cisplatin and fluorouracil/capecitabine for patients with newly diagnosed advanced or metastatic GEJ or gastric cancer whose tumors expressed PD-L1 [defined as Combined Positive Score (CPS) ≥1]. In patients with PD-L1 expression, the addition of pembrolizumab to chemotherapy did not result in an improved mOS when compared to chemotherapy alone (12.5 vs. 11.1 months; HR 0.85, 95% CI, 0.70-1.03) or mPFS (6.9 vs. 6.4 months; HR 0.84, 95% CI, 0.70-1.02) (Tabernero J. et al. (2019) J Clin Oncol 37).

Cancer of the endometrium of the uterus is the most common gynecologic malignancy in resource-abundant countries and the second most common in resource-limited countries (cervical cancer is more common). Endometrioid carcinoma (EMC) is the most common histologic type of endometrial carcinoma and of uterine malignancy overall. Endometrioid tumors tend to have a favorable prognosis and typically present at an early stage with abnormal uterine bleeding. Other histologic types of endometrial carcinoma (e.g., serous, clear cell) as well as other types of uterine cancer are associated with a poor prognosis. For patients presenting with advanced disease, treatment options are limited with no consensus on a standard regimen. Chemotherapy has been the standard of care in the first-line treatment of EMC, with platinum compounds, anthracyclines, and taxanes being the most commonly used, alone and in combination (Colombo, P. E. et al. (2013) Ann Oncol. 2013; 24 (Suppl 6): vi33-8.). Cytotoxic therapy is a treatment option in second-line treatment; however, response rates are low, PFS and OS are short. There are limited subsequent treatment options in relapsed/refractory patients, including: single agent doxorubicin, paclitaxel, pegylated liposomal doxorubicin, and bevacizumab. The median OS in clinical trials after first- or second-line agents is generally 12 months or less.

Despite recent advancements in the field, prognosis and treatment options remain poor for many patients with solid tumors, e.g., gastric cancer, gastroesophageal junction cancer (GEJC), endometrial cancer. As such, there remains a need for safe and effective HER2-targeted combination therapies, particularly for treatment of gastric cancer, gastroesophageal junction cancer (GEJC), and endometrial cancer.

All references cited herein, including patent applications, patent publications, and UniProtKB/Swiss-Prot Accession numbers are herein incorporated by reference in their entirety, as if each individual reference were specifically and individually indicated to be incorporated by reference.

### SUMMARY

In some aspects, provided herein are methods for treating or preventing progression of cancer in an individual, comprising administering to the individual an effective amount of an anti-PD-1 antibody and an anti-human epidermal growth factor receptor 2 (HER2) antibody-drug conjugate that comprises an anti-HER2 antibody and a cytotoxic molecule; wherein the anti-HER2 antibody comprises a heavy chain comprising a heavy chain variable (VH) domain and a light chain comprising a light chain variable (VL) domain; wherein
a) the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence NYLFDH (SEQ ID NO:3), or
b) the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence ARNYLFDHW (SEQ ID NO:12);
   wherein the VL domain comprises a CDR-L1 comprising the amino acid sequence KASQDVGTAVA (SEQ ID NO:4), a CDR-L2 comprising the amino acid sequence WASIRHT (SEQ ID NO:5), and a CDR-L3 comprising the amino acid sequence HQFATYT (SEQ ID NO:6), wherein the cancer is gastric cancer, gastroesophageal junction cancer (GEJC), breast cancer, or endometrial cancer.

In some embodiments, the VH domain comprises the amino acid sequence of SEQ ID NO: 7. In some embodiments according to any of the embodiments described herein, the VL domain comprises the amino acid sequence of SEQ ID NO:8. In some embodiments according to any of the embodiments described herein, the heavy chain comprises the amino acid sequence of SEQ ID NO:9 or SEQ ID NO:10. In some embodiments according to any of the embodiments described herein, the light chain comprises the amino acid sequence of SEQ ID NO: 11.

In some embodiments according to any of the embodiments described herein, the cytotoxic molecule comprises a tubulin inhibitor or DNA damaging agent. In some embodiments, the tubulin inhibitor comprises a dolastatin or derivative thereof, auristatin or derivative thereof, or maytansinoid or derivative thereof. In some embodiments, the tubulin inhibitor comprises monomethyl auristatin E (MMAE), monomethyl auristatin F (MMAF), or auristatin F (AF). In some embodiments, the tubulin inhibitor comprises emtansine (DM1), maytansine (DM3), or ravtansine (DM4). In some embodiments, the DNA damaging agent comprises a calicheamicin, duocarmycin, pyrrolobenzodiazepine (PBD), or SN-38. In some embodiments according to any of the embodiments described herein, the cytotoxic molecule comprises an amanitin, anthracycline, baccatin, camptothecin, cemadotin, colchicine, colcimid, combretastatin, cryptophycin, dicodermolide, docetaxel, doxorubicin, echinomycin, eleutherobin, epothilone, estramustine, lexitropsin, maytansine, methotrexate, netropsin, puromycin, rhizoxins, taxane, tubulysin, or vinca alkaloid. In some embodiments according to any of the embodiments described herein, the antibody-drug conjugate is represented by formula Ab-(L-U) n, wherein Ab is the anti-HER2 antibody, L is a linker between the cytotoxic molecule and the anti-HER2 antibody, U is the conjugated cytotoxic molecule, and n is an integer from 1 to 8, representing the number of cytotoxic molecules bound to the antibody. In some embodiments, the linker is attached to the anti-HER2 antibody via a thiol or amino moiety. In some embodiments, the linker is selected from the group consisting of maleimidocaproyl valine citrulline p-amino-benzyl (mc-vc-pAB), maleimidocaproyl (mc), triglycyl peptide linker, 3-maleimido-propionic acid, Mal-di-EG-OPFP (perfluorophenyl 3-(2-(2-(3-(2,5-dioxo-2,5-dihydro-1H-pyrrol-1-yl) propanamido) ethoxy) ethoxy) propanoate), Mal-di-EG-OSu (2,5-dioxopyrrolidin-1-yl 3-(2-(2-(2,5-dioxo-2,5-dihydro-1H-pyrrol-1-yl) ethoxy) ethoxy) propanoate), Mal-Tri-EG-OSu (2,5-dioxopyrrolidin-1-yl 3-(2-(2-(2-(2,5-dioxo-2,5-dihydro-1H-pyrrol-1-yl) ethoxy) ethoxy) ethoxy) ethoxypropanoate), Mal-Tetra-EG-OSu (2,5-dioxopyrrolidin-1-yl 1-(2,5-dioxo-2,5-dihydro-1H-pyrrol-1-yl)-3-oxo-7,10,13,16-tetraoxa-4-azanonadecan-19-oate), Br-di-EG-OSu (2,5-dioxopyrrolidin-1-yl 3 (2-(2-(2-bromoacetamido) ethoxy) ethoxy) propanoate), Py-ds-prp-OSu (2-5-dioxopyrrolidin-1-yl 3-(pyridine-2-yldisulfanyl) propanoate), Py-ds-Prp-OPEP (perfluorophenyl 3-(pyridine-2-yldisulfanyl) propanoate), Py-ds-dmBut-OSu (2,5-dioxopyrrolidin-1-yl 4-methyl-4-(pyridine-2-yldisulfanyl) pentanoate, Py-ds-dmBut-OPF (perfluorophenyl 4-methyl-4-(pyridine-2-yldisulfanyl) pentanoate), SMCC (N-succinimidyl 4-(maleimidomethyl) cyclohexanecarboxylate), MBS (3-maleimidobenzoic acid N-hydroxysuccinimide ester), SATA (S-(N-succinimidyl)thioacetate), SPDP ((N-succinimidyl 3-(2-pyridyldithio) propionate), and SMPT ((N-succinimidyloxy carbonyl)-1-methyl-1-(2-pyridyldithio) toluene). In some embodiments, the antibody-drug conjugate is disitamab vedotin.

In some embodiments according to any of the embodiments described herein, the anti-PD-1 antibody is toripalimab. In some embodiments according to any of the embodiments described herein, the anti-PD-1 antibody comprises a heavy chain comprising the amino acid sequence of SEQ ID NO:21 and a light chain comprising the amino acid sequence of SEQ ID NO: 22. In some embodiments according to any of the embodiments described herein, comprises a VH domain comprising the amino acid sequence of SEQ ID NO: 19 and a VL domain comprises the amino acid sequence of SEQ ID NO:20.

In some embodiments according to any of the embodiments described herein, the cancer has previously been treated. In some embodiments according to any of the embodiments described herein, (a) prior to administration of anti-PD-1 antibody and the antibody-drug conjugate, the individual has experienced progression on or after one or more standard of care therapies; (b) the individual is intolerant to one or more standard of care therapies; (c) the individual is unable to received treatment; or (d) the standard of care treatment is unavailable.

In some embodiments according to any of the embodiments described herein, the cancer is locally advanced or metastatic.

In some embodiments according to any of the embodiments described herein, cells of the cancer express HER2.

In some embodiments according to any of the embodiments described herein, the cancer is gastric cancer and the method does not comprise administering three additional therapies, wherein the therapies are radiotherapy, GM-CSF, and IL-2.

In some embodiments, the cancer is gastric cancer or gastroesophageal junction cancer (GEJC). In some embodiments, the cancer is gastric adenocarcinoma or gastroesophageal junction adenocarcinoma.

In some embodiments, the cancer is endometrial cancer.

In some embodiments, the cancer is breast cancer.

In some embodiments according to any of the embodiments described herein, the administration of the anti-PD-1 antibody and the antibody-drug conjugate is a second-line (2L) treatment. In some embodiments according to any of the embodiments described herein, the administration of the anti-PD-1 antibody and the antibody-drug conjugate is a third-line or higher (3L+) treatment.

In some embodiments, a sample obtained from the individual comprises cancer cells that show HER2 gene amplification, as measured by in situ hybridization (ISH) assay (ISH-positive).

In some embodiments, the cancer is a HER2-low cancer. In some embodiments, a sample obtained from the individual comprises cancer cells that express HER2 on their cell surface at a level of IHC1+, as measured by immunohistochemistry (IHC) assay. In some embodiments, a sample obtained from the individual comprises cancer cells that express HER2 on their cell surface at a level of IHC2+, as measured by IHC assay; and the sample obtained from the individual comprises cancer cells that do not exhibit HER2 gene amplification, as measured by in situ hybridization (ISH) assay (ISH-negative).

In some embodiments, the cancer is a HER2-positive cancer. In some embodiments, a sample obtained from the individual comprises cancer cells that express HER2 on their cell surface at a level of IHC3+, as measured by IHC assay. In some embodiments, a sample obtained from the individual comprises cancer cells that express HER2 on their cell surface at a level of IHC2+, as measured by IHC assay; and the sample obtained from the individual comprises cancer cells that exhibit HER2 gene amplification, as measured by ISH assay (ISH-positive).

In some embodiments, the cancer is a HER2-positive/HER2-low, locally advanced or metastatic gastric cancer, or a HER2-positive/HER2-low locally advanced or metastatic gastroesophageal junction cancer.

In some embodiments, the cancer is a hormone receptor (HR)-negative, HER2-low expressing, unresectable locally advanced or metastatic breast cancer; a standard therapy progressed HR-positive, HER2-low expressing, unresectable locally advanced or metastatic breast cancer; a HR+HER2-low expressing breast cancer wherein the individual had received at least one line of chemotherapy; or a HR-HER2-low expressing breast cancer wherein the individual had received at least one line of chemotherapy.

In some embodiments according to any of the embodiments described herein, the individual has a Combined Positive Score (CPS) ≥1.

In some embodiments according to any of the embodiments described herein, the antibody-drug conjugate is administered to the individual at a dose of 2.0 mg/kg. In some embodiments according to any of the embodiments described herein, the antibody-drug conjugate is administered to the individual at a dose of 2.5 mg/kg. In some embodiments, the dose is measured using the bovine serum albumin (BSA)-based Extinction Coefficient (EC) method.

In some embodiments according to any of the embodiments described herein, the antibody-drug conjugate is administered intravenously to the individual. In some embodiments according to any of the embodiments described herein, the antibody-drug conjugate is administered to the individual every 2 weeks or every 14 days. In some embodiments according to any of the embodiments described herein, the anti-PD-1 antibody is administered to the individual at a dose of 3.0 mg/kg.

In some embodiments according to any of the embodiments described herein, anti-PD-1 antibody is administered intravenously to the individual. In some embodiments according to any of the embodiments described herein, the anti-PD-1 antibody is administered to the individual every 2 weeks or every 14 days.

In some embodiments according to any of the embodiments described herein, the antibody-drug conjugate is administered sequentially with the anti-PD-1 antibody. In some embodiments according to any of the embodiments described herein, the antibody-drug conjugate is administered prior to the anti-PD-1 antibody. In some embodiments according to any of the embodiments described herein, the interval between administration of the antibody-drug conjugate and the anti-PD-1 antibody is 30 to 60 minutes. In some embodiments, the method comprises intravenously administering disitamab vedotin to the individual at 2.5 mg/kg and intravenously administering toripalimab to the individual at 3.0 mg/kg, every 2 weeks or every 14 days, on the same day of each cycle, wherein the disitamab vedotin is administered prior to toripalimab, the interval between administration is 30 to 60 minutes, and the dose of disitamab vedotin is measured using the bovine serum albumin (BSA)-based Extinction Coefficient (EC) method.

In some embodiments according to any of the embodiments described herein, administration of the anti-PD-1 antibody and the antibody-drug conjugate results in a complete response (CR) or partial response (PR) in the individual. In some embodiments, administration of the anti-PD-1 antibody and the antibody-drug conjugate to an individual with gastric cancer or GEJC results in an overall survival (OS) of 14 months or longer. In some embodiments, administration of the anti-PD-1 antibody and the antibody-drug conjugate to an individual with gastric cancer or GEJC results in progression-free survival (PFS) of 5 months or longer.

In some embodiments, the administration of the anti-PD-1 antibody and the antibody-drug conjugate to an individual with breast cancer results in progression-free survival (PFS) of 9 months or longer. In some embodiments, the breast cancer is hormone receptor (HR)+HER2-low expressing breast cancer, and the method comprises intravenously administering disitamab vedotin to the individual at 2.5 mg/kg and intravenously administering toripalimab to the individual at 3.0 mg/kg, every 2 weeks or every 14 days, on the same day of each cycle.

In some embodiments, the administration of the anti-PD-1 antibody and the antibody-drug conjugate to an individual with breast cancer results in progression-free survival (PFS) of 4 months or longer. In some embodiments, the breast cancer is hormone receptor (HR)+HER2-low expressing breast cancer, and the method comprises intravenously administering disitamab vedotin to the individual at 2.0 mg/kg and intravenously administering toripalimab to the individual at 3.0 mg/kg, every 2 weeks or every 14 days, on the same day of each cycle.

In some embodiments, the method described herein for treating cancer comprises administering an anti-PD-1 antibody, HER2 antibody-drug conjugate, and a chemotherapy. In some embodiments, the chemotherapy comprises oxaliplatin and capecitabine (CAPOX). In some embodiments, the HER2 antibody-drug conjugate is disitamab vedotin. In some embodiments, the anti-PD-1 antibody is toripalimab, or an antibody with the VH and VL sequences of toripalimab. In some embodiments, disitamab vedotin, toripalimab, oxaliplatin, and capecitabine are administered in a 6-week or 42-day cycle. In some embodiments, disitamab vedotin, toripalimab, oxaliplatin, and capecitabine are administered starting on day 1 of the 6-week or 42-day cycle. In some embodiments, disitamab vedotin is administered at a dose of 2.5 mg/kg intravenously once every 2 weeks (Q2W). In some embodiments, disitamab vedotin is administered at a dose of 2.0 mg/kg intravenously once every 2 weeks (Q2W). In some embodiments, toripalimab is intravenously administered at a dose of 3 mg/kg once every 2 weeks (Q2W). In some embodiments, oxaliplatin is administered at a dose of 100 mg/m² intravenously once every 3 weeks or every 21 days (Q3W). In some embodiments, the capecitabine is administered at a dose of 750 mg/m² orally once daily on days 1, 15, 22, and 36 of the 6-week or 42-day cycle; and the capecitabine is administered at a dose of 750 mg/m² orally twice daily on days 2-14 and 23-35 of the 6-week or 42-day cycle. In some embodiments, the capecitabine is administered at a dose of 1000 mg/m² orally once daily on days 1, 15, 22, and 36 of the 6-week or 42-day cycle; and the capecitabine is administered at a dose of 1000 mg/m² orally twice daily on days 2-14 and 23-35 of the 6-week or 42-day cycle. In some embodiments, disitamab vedotin, toripalimab, oxaliplatin, and capecitabine are administered sequentially on day 1 of the 6-week or 42-day cycle. In some embodiments, the method comprises administering the treatment on day 1 in the order: disitamab vedotin, then toripalimab, then oxaliplatin, then capecitabine. In some embodiments, the interval between end of the administration of toripalimab and start of the administration of oxaliplatin is 60 minutes. In some embodiments, the interval between two consecutive administrations of capecitabine is between about 10 hours and about 12 hours. In some embodiments, the dose of disitamab vedotin is measured using the bovine serum albumin (BSA)-based Extinction Coefficient (EC) method. In some embodiments, disitamab vedotin, toripalimab, oxaliplatin, and capecitabine are administered in a 6-week or 42-day cycle; wherein disitamab vedotin, toripalimab, oxaliplatin, and capecitabine are administered starting on day 1 of a 6-week or 42-day cycle; wherein disitamab vedotin is administered at a dose of 2.5 mg/kg intravenously once every 2 weeks (Q2W); wherein toripalimab is intravenously administered at a dose of 3 mg/kg once every 2 weeks (Q2W); wherein oxaliplatin is administered at a dose of 100 mg/m² intravenously once every 3 weeks or every 21 days (Q3W); wherein the capecitabine is administered at a dose of 750 mg/m² orally once on days 1, 15, 22, and 36 of the 6-week or 42-day cycle; wherein the capecitabine is administered at a dose of 750 mg/m² orally twice daily on days 2-14 and 23-35 of the 6-week or 42-day cycle; and wherein the dose of disitamab vedotin is measured using the bovine serum albumin (BSA)-based Extinction Coefficient (EC) method. In some embodiments, disitamab vedotin, toripalimab, oxaliplatin, and capecitabine are administered in a 6-week or 42-day cycle; wherein disitamab vedotin, toripalimab, oxaliplatin, and capecitabine are administered starting on day 1 of a 6-week or 42-day cycle; wherein disitamab vedotin is administered at a dose of 2.5 mg/kg intravenously once every 2 weeks (Q2W); wherein toripalimab is intravenously administered at a dose of 3 mg/kg once every 2 weeks (Q2W); wherein oxaliplatin is administered at a dose of 100 mg/m² intravenously once every 3 weeks or every 21 days (Q3W); wherein the capecitabine is administered at a dose of 1000 mg/m² orally once on days 1, 15, 22, and 36 of the 6-week or 42-day cycle; wherein the capecitabine is administered at a dose of 1000 mg/m² orally twice daily on days 2-14 and 23-35 of the 6-week or 42-day cycle; and wherein the dose of disitamab vedotin is measured using the bovine serum albumin (BSA)-based Extinction Coefficient (EC) method. In some embodiments, disitamab vedotin, toripalimab, oxaliplatin, and capecitabine are administered starting in a 6-week or 42-day cycle; wherein disitamab vedotin, toripalimab, oxaliplatin, and capecitabine are administered starting on day 1 of a 6-week or 42-day cycle; wherein disitamab vedotin is administered at a dose of 2.0 mg/kg intravenously once every 2 weeks (Q2W); wherein toripalimab is intravenously administered at a dose of 3 mg/kg once every 2 weeks (Q2W); wherein oxaliplatin is administered at a dose of 100 mg/m² intravenously once every 3 weeks or every 21 days (Q3W); wherein the capecitabine is administered at a dose of 750 mg/m² orally once on days 1, 15, 22, and 36 of the 6-week or 42-day cycle; wherein the capecitabine is administered at a dose of 750 mg/m² orally twice daily on days 2-14 and 23-35 of the 6-week or 42-day cycle; and wherein the dose of disitamab vedotin is measured using the bovine serum albumin (BSA)-based Extinction Coefficient (EC) method. In some embodiments, disitamab vedotin, toripalimab, oxaliplatin, and capecitabine are administered in a 6-week or 42-day cycle; wherein disitamab vedotin, toripalimab, oxaliplatin, and capecitabine are administered starting on day 1 of a 6-week or 42-day cycle; wherein disitamab vedotin is administered at a dose of 2.0 mg/kg intravenously once every 2 weeks (Q2W); wherein toripalimab is intravenously administered at a dose of 3 mg/kg once every 2 weeks (Q2W); wherein oxaliplatin is administered at a dose of 100 mg/m² intravenously once every 3 weeks or every 21 days (Q3W); wherein the capecitabine is administered at a dose of 1000 mg/m² orally once on days 1, 15, 22, and 36 of the 6-week or 42-day cycle; wherein the capecitabine is administered at a dose of 1000 mg/m² orally twice daily on days 2-14 and 23-35 of the 6-week or 42-day cycle; and wherein the dose of disitamab vedotin is measured using the bovine serum albumin (BSA)-based Extinction Coefficient (EC) method. In some embodiments, disitamab vedotin, toripalimab, oxaliplatin, and capecitabine are administered at the dose and intervals as described in Example 6.

In some embodiments, the administration of the anti-PD-1 antibody, the antibody-drug conjugate, and the chemotherapy is a first-line (1L) treatment. In some embodiments, the cancer is a HER2-zero cancer. In some embodiments, a sample obtained from the individual comprises cancer cells that do not express HER2; or a sample obtained from the individual expresses HER2 on their cell surface at a level of IHC-0, as measured by IHC assay. In some embodiments, a sample obtained from the individual comprises cancer cells that show HER2 gene amplification, as measured by in situ hybridization (ISH) assay (ISH-positive). In some embodiments, the cancer is a HER2-low cancer. In some embodiments, a sample obtained from the individual comprises cancer cells that express HER2 on their cell surface at a level of IHC1+, as measured by immunohistochemistry (IHC) assay. In some embodiments, a sample obtained from the individual comprises cancer cells that express HER2 on their cell surface at a level of IHC2+, as measured by IHC assay; and wherein the sample obtained from the individual comprises cancer cells that do not exhibit HER2 gene amplification, as measured by in situ hybridization (ISH) assay (ISH-negative). In some embodiments, the cancer is a HER2-positive cancer. In some embodiments, a sample obtained from the individual comprises cancer cells that express HER2 on their cell surface at a level of IHC3+, as measured by IHC assay. In some embodiments, a sample obtained from the individual comprises cancer cells that express HER2 on their cell surface at a level of IHC2+, as measured by IHC assay; and wherein the sample obtained from the individual comprises cancer cells that exhibit HER2 gene amplification, as measured by ISH assay (ISH-positive). In some embodiments, the cancer is a HER2-positive, HER2-low, or HER2-zero locally advanced or metastatic gastric cancer; or a HER2-positive, HER2-low, or HER2-zero locally advanced or metastatic gastroesophageal junction adenocarcinoma. In some embodiments, the individual has a PD-L1 Combined Positive Score (CPS) ≥1 or CPS <1.

In some embodiments, administration of the anti-PD-1 antibody, the antibody-drug conjugate, and the chemotherapy to a population of individuals with HER2-positive gastric cancer or GEJC results in an overall response rate of 73% or greater. In some embodiments, administration of the anti-PD-1 antibody, the antibody-drug conjugate, and the chemotherapy to a population of individuals with HER2-low gastric cancer or GEJC results in an overall response rate of 67% or greater.

In some embodiments, the method described herein for treating cancer comprises administering an anti-PD-1 antibody, a HER2 antibody-drug conjugate, and an anti-HER2 antibody. In some embodiments, the ani-HER2 antibody is trastuzumab, or an antibody with the VH and VL sequences of trastuzumab. In some embodiments, the HER2 antibody-drug conjugate is disitamab vedotin. In some embodiments, the anti-PD-1 antibody is toripalimab. In some embodiments, disitamab vedotin, toripalimab, and trastuzumab are administered in a 6-week or 42-day cycle. In some embodiments, disitamab vedotin, toripalimab, and trastuzumab are administered on day 1 of a 6-week or 42-day cycle. In some embodiments, disitamab vedotin is administered at a dose of 2.5 mg/kg intravenously once every 2 weeks (Q2W). In some embodiments, toripalimab is administered at a dose of 3 mg/kg intravenously once every 2 weeks (Q2W). In some embodiments, trastuzumab is administered at an initial dose of 8 mg/kg on day 1 of the 6-week or 42-day cycle; and trastuzumab is administered at a dose of about 6 mg/kg every 3 weeks (Q3W) starting about week 3 of the 6-week or 42-day cycle. In some embodiments, the dose of disitamab vedotin is measured using the bovine serum albumin (BSA)-based Extinction Coefficient (EC) method. In some embodiments, disitamab vedotin, toripalimab, and trastuzumab are administered sequentially on day 1 of the 6-week or 42-day cycle. In some embodiments, the method comprises administering the treatment on day 1 in the order: disitamab vedotin, then toripalimab, then trastuzumab. In some embodiments, the interval between end of the administration of toripalimab and start of the administration of oxaliplatin is 60 minutes. In some embodiments, the interval between two consecutive administrations of capecitabine is between about 10 hours and about 12 hours. In some embodiments, disitamab vedotin, toripalimab, and trastuzumab are administered in a 6-week or 42-day cycle; wherein disitamab vedotin, toripalimab, and trastuzumab are administered starting on day 1 of a 6-week or 42-day cycle; wherein disitamab vedotin is administered at a dose of 2.5 mg/kg intravenously once every 2 weeks (Q2W); wherein toripalimab is administered at a dose of 3 mg/kg intravenously once every 2 weeks (Q2W); wherein trastuzumab is administered at an initial dose of 8 mg/kg on day 1 of the 6-week or 42-day cycle; wherein trastuzumab is administered at a dose of about 6 mg/kg every 3 weeks (Q3W) starting about week 3 of the 6-week or 42-day cycle; and wherein the dose of disitamab vedotin is measured using the bovine serum albumin (BSA)-based Extinction Coefficient (EC) method. In some embodiments, disitamab vedotin, toripalimab, and trastuzumab are administered at the dose and intervals as described in Example 6.

In some embodiments, the administration of the anti-PD-1 antibody, the antibody-drug conjugate, and the anti-HER2 antibody is a first-line (1L) treatment. In some embodiments, a sample obtained from the individual comprises cancer cells that show HER2 gene amplification, as measured by in situ hybridization (ISH) assay (ISH-positive). In some embodiments, the cancer is a HER2-positive cancer. In some embodiments, a sample obtained from the individual comprises cancer cells that express HER2 on their cell surface at a level of IHC3+, as measured by IHC assay. In some embodiments, a sample obtained from the individual comprises cancer cells that express HER2 on their cell surface at a level of IHC2+, as measured by IHC assay; and wherein the sample obtained from the individual comprises cancer cells that exhibit HER2 gene amplification, as measured by ISH assay (ISH-positive). In some embodiments, the cancer is a HER2-positive, locally advanced or metastatic gastric cancer; or a HER2-positive locally advanced or metastatic gastroesophageal junction adenocarcinoma. In some embodiments, the individual has a PD-L1 Combined Positive Score (CPS) ≥1 or CPS <1.

In some embodiments, administration of the anti-PD-1 antibody, the antibody-drug conjugate, and the anti-HER2 antibody to a population of individuals with HER2-positive gastric cancer or GEJC results in an overall response rate of 94% or greater.

In some embodiments according to any of the embodiments described herein, the individual is a human.

In some aspects, provided herein are compositions comprising an anti-HER2 antibody-drug conjugate for use in a method of treating or preventing progression of cancer in an individual, wherein the method comprises administering an effective amount of an anti-PD-1 antibody and the anti-HER2 antibody-drug conjugate according to the methods of any of the embodiments described herein. In some embodiments, the method further comprises administering an effective amount of an anti-HER2 antibody. In some embodiments, the anti-HER2 antibody is trastuzumab. In some embodiments, the method further comprises administering an effective amount of a chemotherapy. In some embodiments, the chemotherapy comprises oxaliplatin and capecitabine.

In some aspects, provided herein are combinations comprising an anti-HER2 antibody-drug conjugate and an anti-PD-1 antibody for use in a method of treating or preventing progression of cancer in an individual, wherein the method comprises administering an effective amount of the combination according to the methods of any of the embodiments described herein. In some embodiments, the combination further comprises an anti-HER2 antibody. In some embodiments, the anti-HER2 antibody is trastuzumab. In some embodiments, the combination further comprises a chemotherapy. In some embodiments, the chemotherapy is oxaliplatin and capecitabine.

In some aspects, provided herein are kits comprising an anti-PD-1 antibody and an anti-HER2 antibody-drug conjugate that comprises an anti-HER2 antibody and a cytotoxic molecule; wherein the anti-HER2 antibody comprises a heavy chain comprising a heavy chain variable (VH) domain and a light chain comprising a light chain variable (VL) domain; wherein (i) the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence NYLFDH (SEQ ID NO:3), or (ii) the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence ARNYLFDHW (SEQ ID NO:12); wherein the VL domain comprises a CDR-L1 comprising the amino acid sequence KASQDVGTAVA (SEQ ID NO:4), a CDR-L2 comprising the amino acid sequence WASIRHT (SEQ ID NO:5), and a CDR-L3 comprising the amino acid sequence HQFATYT (SEQ ID NO:6). In some embodiments, the kit further comprises instructions for administering an effective amount of an anti-PD-1 antibody and the antibody-drug conjugate to an individual in need thereof according to the methods of any of the embodiments described herein. In some embodiments, the kit further comprises an anti-HER2 antibody. In some embodiments, the anti-HER2 antibody is trastuzumab. In some embodiments, the kit further comprises a chemotherapy. In some embodiments, the chemotherapy comprises oxaliplatin and capecitabine.

In some aspects, provided herein are kits comprising (a) an anti-HER2 antibody-drug conjugate that comprises an anti-HER2 antibody and a cytotoxic molecule; wherein the anti-HER2 antibody comprises a heavy chain comprising a heavy chain variable (VH) domain and a light chain comprising a light chain variable (VL) domain; wherein (i) the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence NYLFDH (SEQ ID NO:3), or (ii) the VH domain comprises a CDR-H1 comprising the amino acid sequence DYYIH (SEQ ID NO:1), a CDR-H2 comprising the amino acid sequence RVNPDHGDSYYNQKFKD (SEQ ID NO: 2), and a CDR-H3 comprising the amino acid sequence ARNYLFDHW (SEQ ID NO: 12); wherein the VL domain comprises a CDR-L1 comprising the amino acid sequence KASQDVGTAVA (SEQ ID NO:4), a CDR-L2 comprising the amino acid sequence WASIRHT (SEQ ID NO:5), and a CDR-L3 comprising the amino acid sequence HQFATYT (SEQ ID NO:6); and (b) instructions for administering an effective amount of the antibody-drug conjugate and an anti-PD-1 antibody to an individual in need thereof according to the methods of any of the embodiments described herein. In some embodiments, the instructions further instruct administering an effective amount of an anti-HER2 antibody. In some embodiments, the anti-HER2 antibody is trastuzumab. In some embodiments, the instructions further instruct administering an effective amount of a chemotherapy. In some embodiments, the chemotherapy comprises oxaliplatin and capecitabine.

In some aspects, provided herein are methods for treating or preventing progression of cancer in an individual, comprising administering to the individual an effective amount of an anti-PD-1 antibody and an anti-human epidermal growth factor receptor 2 (HER2) antibody-drug conjugate that comprises an anti-HER2 antibody and a cytotoxic molecule; wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO:1), a CDR-H2 comprising the amino acid sequence RVNPDHGDSYYNQKFKD (SEQ ID NO: 2), and a CDR-H3 comprising the amino acid sequence ARNYLFDHW (SEQ ID NO: 12); and wherein the VL domain comprises a CDR-L1 comprising the amino acid sequence KASQDVGTAVA (SEQ ID NO:4), a CDR-L2 comprising the amino acid sequence WASIRHT (SEQ ID NO:5), and a CDR-L3 comprising the amino acid sequence HQFATYT (SEQ ID NO:6), wherein the cancer is gastric cancer, gastroesophageal junction cancer (GEJC), or endometrial cancer.

In some aspects, provided herein are kits comprising an anti-PD-1 antibody and an anti-HER2 antibody-drug conjugate that comprises an anti-HER2 antibody and a cytotoxic molecule; wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence ARNYLFDHW (SEQ ID NO:12); wherein the VL domain comprises a CDR-L1 comprising the amino acid sequence KASQDVGTAVA (SEQ ID NO:4), a CDR-L2 comprising the amino acid sequence WASIRHT (SEQ ID NO:5), and a CDR-L3 comprising the amino acid sequence HQFATYT (SEQ ID NO:6).

In some aspects, provided herein are kits comprising (a) an anti-HER2 antibody-drug conjugate that comprises an anti-HER2 antibody and a cytotoxic molecule; wherein the anti-HER2 antibody comprises a heavy chain comprising a heavy chain variable (VH) domain and a light chain comprising a light chain variable (VL) domain; wherein the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence ARNYLFDHW (SEQ ID NO:12); wherein the VL domain comprises a CDR-L1 comprising the amino acid sequence KASQDVGTAVA (SEQ ID NO:4), a CDR-L2 comprising the amino acid sequence WASIRHT (SEQ ID NO:5), and a CDR-L3 comprising the amino acid sequence HQFATYT (SEQ ID NO:6); and (b) instructions for administering an effective amount of the antibody-drug conjugate and an anti-PD-1 antibody to an individual in need thereof according to the methods of any of the embodiments described herein.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1 shows a schematic of the dose-limiting evaluation design, per the clinical trial described in Examples 1, 2, and 5. RC48-ADC: DV.

FIG. 2 shows the overall design of the phase II clinical study described in Examples 5 and 6, evaluating the efficacy and safety of RC48-ADC in combination with toripalimab and chemotherapy or RC48-ADC in combination with toripalimab and trastuzumab in first-line treatment of patients with HER2-expressing locally advanced or metastatic gastric cancer (including adenocarcinoma of the gastroesophageal junction).

FIG. 3 shows the overall design of the phase III clinical study described in Examples 5 and 6, evaluating the efficacy and safety of RC48-ADC in combination with toripalimab and chemotherapy or RC48-ADC in combination with toripalimab and trastuzumab in first-line treatment of patients with HER2-expressing locally advanced or metastatic gastric cancer (including adenocarcinoma of the gastroesophageal junction).

### DETAILED DESCRIPTION

### I. Definitions

Before describing the invention in detail, it is to be understood that this invention is not limited to particular compositions or biological systems, which can, of course, vary. It is also to be understood that the terminology used herein is for the purpose of describing particular embodiments only, and is not intended to be limiting.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure. For purposes of the present disclosure, the following terms are defined.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “about” and “approximately” as used herein shall generally mean an acceptable degree of error for the quantity measured given the nature or precision of the measurements. Typical, exemplary degrees of error are within 20 percent (%), preferably within 10%, and more preferably within 5% of a given value or range of values. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.” The terms “about” and “approximately,” particularly in reference to a given quantity, encompass and describe the given quantity itself.

Alternatively, in biological systems, the terms “about” and “approximately” may mean values that are within an order of magnitude, preferably within 5-fold, and more preferably within 2-fold of a given value. Numerical quantities given herein are approximate unless stated otherwise, meaning that the term “about” or “approximately” can be inferred when not expressly stated.

When “about” is applied to the beginning of a numerical range, it applies to both ends of the range. Thus, “from about 5 to 20%” is equivalent to “from about 5% to about 20%” When “about” is applied to the first value of a set of values, it applies to all values in that set. Thus, “about 7, 9, or 11 mg/kg” is equivalent to “about 7, about 9, or about 11 mg/kg.”

“Administering” or “administration” refer to the physical introduction of a therapeutic agent to a subject, using any of the various methods and delivery systems known to those skilled in the art. Exemplary routes of administration include oral, intravenous, intramuscular, subcutaneous, intraperitoneal, spinal or other parenteral routes of administration, for example by injection or infusion (e.g intravenous infusion). The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administration can also be performed, for example, once, a plurality of times, and/or over one or more extended periods.

As used herein, the term “administering” when referring to two or more components (e.g., an anti-PD-1 antibody and an anti-HER2 antibody-drug conjugate) includes sequential or simultaneous administration of the anti-PD-1 antibody and the anti-HER2 antibody-drug conjugate. In some instances, the co-administered compounds are administered via different routes. For example, one or two compounds can be administered orally, and the other compound(s) can be administered, e.g, sequentially or simultaneously, via intravenous, intramuscular, subcutaneous, or intraperitoneal injection. The simultaneously or sequentially administered compounds or compositions can be administered such that the anti-HER2 antibody-drug conjugate and the anti-PD-1 antibody are simultaneously present in a subject or in a cell at an effective concentration.

“Simultaneous administration,” as used herein, means that the two or more therapies (e.g., in a combination therapy) are administered with a time separation of no more than about 15 minutes, such as no more than about any of 10, 5, or 1 minutes. When the two or more therapies are administered simultaneously, the two or more therapies can be contained in the same composition (e.g., a composition comprising both a first and second therapy) or in separate compositions (e.g., a first therapy in one composition and a second therapy is contained in another composition).

As used herein, the term “sequential administration” means that the two or more therapies (e.g., in a combination therapy) are administered with a time separation of more than about 15 minutes, such as more than about any of 20, 30, 40, 50, 60, or more minutes. Any of the two or more therapies may be administered first. The two or more therapies are contained in separate compositions, which may be contained in the same or different packages or kits.

As used herein, the term “concurrent administration” means that the administration of two or more therapies (e.g., in a combination therapy) overlap with each other. For example, the two or more therapies may be administered in the same day, or with a time separation of within one day, within two days, within three days, within four days, within five days, within six days, within seven days, within ten days, within fourteen days, or within twenty-one days.

A “cancer” refers to a broad group of various diseases characterized by the uncontrolled growth of abnormal cells in the body. A “cancer” or “cancer tissue” can include a tumor.

The term “metastasis” is an art known term that refers to the spread of cancer cells from the place where they first formed (the primary site) to one or more other sites in a subject (one or more secondary sites). In metastasis, cancer cells break away from the original (primary) tumor, travel through the blood or lymph system, and form a new tumor (a metastatic tumor) in other organs or tissues of the body. The new, metastatic tumor includes the same or similar cancer cells as the primary tumor. At the secondary site, the tumor cell may proliferate and begin the growth or colonization of a secondary tumor at this distant site.

The term “HER2” (also known as also known as HER2/neu, ERBB2, CD340, receptor tyrosine-protein kinase erbB-2, proto-oncogene Neu, and human epidermal growth factor receptor 2) refers to a member of the human epidermal growth factor receptor (HER/EGFR/ERBB) family of receptor tyrosine kinases. Amplification or overexpression of HER2 plays a significant role in the development and progression of certain aggressive types of cancer, including colorectal cancer, gastric cancer, lung cancer (e.g non-small cell lung cancer (NSCLC)), biliary cancers (e.g., cholangiocarcinoma, gallbladder cancer), bladder cancer, esophageal cancer, melanoma, ovarian cancer, liver cancer, prostate cancer, pancreatic cancer, small intestine cancer, head and neck cancer, uterine cancer, cervical cancer, and breast cancer. Non-limiting examples of HER2 nucleotide sequences are set forth in GenBank reference numbers NP_001005862, NP_001289936, NP_001289937, NP_001289938, and NP_004448. Non-limiting examples of HER2 peptide sequences are set forth in GenBank reference numbers NP OO 1005862, NP_001276865, NP_001276866, NP_001276867, and NP_004439.

The term “anti-HER2 antibody-drug conjugate” refers to an anti-HER2 antibody conjugated to a therapeutic agent {i.e., a drug or cytotoxic molecule) optionally via a linker.

An “anti-HER2 antibody”, as used herein, refers to an antibody that binds to the HER2 protein. Anti-HER2 antibodies used for the treatment of cancer are typically monoclonal, although polyclonal antibodies are not excluded by the term. Anti-HER2 antibodies inhibit HER2 activation or downstream signaling by various mechanisms. As non-limiting examples, anti-HER2 antibodies can prevent ligand binding, receptor activation or receptor signal propagation, result in reduced HER2 expression or localization to the cell surface, inhibit HER2 cleavage, or induce antibody-mediated cytotoxicity. Non-limiting examples of anti-HER2 antibodies that are suitable for use in the methods and compositions of the present invention include hertuzumab, trastuzumab, pertuzumab, margetuximab, and combinations thereof.

“Treatment” or “therapy” of a subject refers to any type of intervention or process performed on, or the administration of an active agent to, the subject with the objective of reversing, alleviating, ameliorating, inhibiting, slowing down, or preventing the onset, progression, development, severity, or recurrence of a symptom, complication, condition, or biochemical indicia associated with a disease. In some embodiments, the disease is cancer. As used herein, the terms “treatment” and “treating” when referring, e.g., to the treatment of a cancer, are not intended to be absolute terms. For example, “treatment of cancer” and “treating cancer”, as used in a clinical setting, is intended to include obtaining beneficial or desired clinical results and can include an improvement in the condition of a subject having cancer. Beneficial or desired clinical results include, but are not limited to, one or more of the following: reducing the proliferation of (or destroying) neoplastic or cancerous cells, inhibiting metastasis of neoplastic cells, a decrease in metastasis in a subject, shrinking or decreasing the size of a tumor, change in the growth rate of one or more tumor(s) in a subject, an increase in the period of remission for a subject (e.g., as compared to the one or more metric(s) in a subject having a similar cancer receiving no treatment or a different treatment, or as compared to the one or more metric(s) in the same subject prior to treatment), decreasing symptoms resulting from a disease, increasing the quality of life of those suffering from a disease (e.g., assessed using FACT-G or EORTC-QLQC30), decreasing the dose of other medications required to treat a disease, delaying the progression of a disease, and/or prolonging survival of subjects having a disease. The term “prophylactic” or “prophylactically” refers to any type of intervention or process performed on, or the administration of an active agent to, the subject with the objective of protecting or preventing a disease or condition from developing or at least not developing fully (e.g., to reduce the symptoms or severity of the disease or condition) such as in the development of a side effect (e.g., diarrhea).

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human. The terms “subject” and “patient” and “individual” are used interchangeably herein.

An “effective amount” or “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example, an “anti-cancer agent” promotes cancer regression in a subject. In some embodiments, a therapeutically effective amount of the drug promotes cancer regression to the point of eliminating the cancer. “Promoting cancer regression” means that administering an effective amount of the drug, alone or in combination with an anti-cancer agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity or other adverse physiological effects at the cellular, organ and/or organism level (adverse effects) resulting from administration of the drug.

The phrase “pharmaceutically acceptable” indicates that the substance or composition must be compatible chemically and/or toxicologically, with the other ingredients comprising a formulation, and/or the mammal being treated therewith.

As used herein, the term “pharmaceutically acceptable carrier” refers to a substance that aids the administration of an active agent to a cell, an organism, or a subject. “Pharmaceutically acceptable carrier” refers to a carrier or excipient that can be included in the compositions of the disclosure and that causes no significant adverse toxicological effect on the subject. Non-limiting examples of pharmaceutically acceptable carriers include water, NaCl, normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also be substances for providing the formulation with stability, sterility and isotonicity (e.g., antimicrobial preservatives, antioxidants, chelating agents and buffers), for preventing the action of microorganisms (e.g. antimicrobial and antifungal agents, such as parabens, chlorobutanol, phenol, sorbic acid and the like) or for providing the formulation with an edible flavor etc. In some instances, the carrier is an agent that facilitates the delivery of a small molecule drug or antibody to a target cell or tissue. One of skill in the art will recognize that other pharmaceutical carriers are useful in the present disclosure.

The phrase “pharmaceutically acceptable salt” as used herein, refers to pharmaceutically acceptable organic or inorganic salts of a compound of the disclosure. Exemplary salts include, but are not limited, to sulfate, citrate, acetate, oxalate, chloride, bromide, iodide, nitrate, bi sulfate, phosphate, acid phosphate, isonicotinate, lactate, salicylate, acid citrate, tartrate, oleate, tannate, pantothenate, bitartrate, ascorbate, succinate, maleate, gen isinate, fumarate, gluconate, glucuronate, saccharate, formate, benzoate, glutamate, methanesulfonate “mesylate”, ethanesulfonate, benzenesulfonate, p-toluenesulfonate, pamoate (i.e., 4,4′-methyl ene-bis-(2-hydroxy-3-napbthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

### II. Methods of Treating Cancer

Provided herein is a method of treating or preventing progression of cancer (e.g., gastric cancer, gastroesophageal junction cancer (GEJC), breast cancer, endometrial cancer) in an individual, comprising administering to the individual an effective amount of an anti-PD-1 antibody and an anti-human epidermal growth factor receptor 2 (HER2) antibody-drug conjugate. In some embodiments, the cancer is gastric cancer, gastroesophageal junction cancer (GEJC), or endometrial cancer. In some embodiments, the cancer has previously been treated. In some embodiments, the anti-PD-1 antibody is toripalimab. In some embodiments, the HER2 antibody-drug conjugate is disitamab vedotin. In some embodiments, the cancer has previously been treated. In some embodiments, the administration of the anti-PD-1 antibody and the antibody-drug conjugate is a second-line (2L) treatment. In some embodiments, the administration of the anti-PD-1 antibody and the antibody-drug conjugate is a third-line or higher (3L+) treatment. In some embodiments, the cancer is endometrial cancer. In some embodiments, the cancer is a HER2-positive/HER2-low, locally advanced or metastatic gastric cancer; or a HER2-positive/HER2-low locally advanced or metastatic gastroesophageal junction cancer. In some embodiments, the cancer is HER2-low. In some embodiments, the cancer is a hormone receptor (HR)-negative, HER2-low expressing, unresectable locally advanced or metastatic breast cancer; or a standard therapy progressed HR-positive, HER2-low expressing, unresectable locally advanced or metastatic breast cancer. In some embodiments, the cancer is HER2-positive. In some embodiments, the cancer is gastric cancer, and the method does not comprise administering three additional therapies, wherein the therapies are radiotherapy, GM-CSF, and IL-2. In some embodiments, the method does not comprise administering chemotherapy, e.g., oxaliplatin or capecitabine. In some embodiments, the anti-HER2 antibody-drug conjugate comprises an anti-HER2 antibody and a cytotoxic molecule (e.g., wherein one or more cytotoxic molecule(s) is/are linked the anti-HER2 antibody directly or via linker).

Provided herein is a method of treating or preventing progression of cancer, e.g., gastric cancer or GEJC (e.g., gastroesophageal junction adenocarcinoma) wherein the method comprises administering to the individual an effective amount of an anti-PD-1 antibody and an HER2 antibody-drug conjugate and a chemotherapy. In some embodiments, the chemotherapy comprises capecitabine and oxaliplatin (CAPOX). In some embodiments, the anti-PD-1 antibody is toripalimab. In some embodiments, the HER2 antibody-drug conjugate is disitamab vedotin. In some embodiments, the anti-HER2 antibody-drug conjugate comprises an anti-HER2 antibody and a cytotoxic molecule (e.g., wherein one or more cytotoxic molecule(s) is/are linked the anti-HER2 antibody directly or via linker). In some embodiments, the administration of the anti-PD-1 antibody, the antibody-drug conjugate, and the chemotherapy is a first-line (1L) treatment. In some embodiments, the cancer is HER2-low. In some embodiments, the cancer is HER2-positive.

Provided herein is a method of treating or preventing progression of cancer, e.g., gastric cancer or GEJC (e.g., gastroesophageal junction adenocarcinoma) wherein the method comprises administering to the individual an effective amount of an anti-PD-1 antibody and an HER2 antibody-drug conjugate and a HER2 antibody. In some embodiments, the anti-HER2 antibody is trastuzumab. In some embodiments, the anti-PD-1 antibody is toripalimab. In some embodiments, the HER2 antibody-drug conjugate is disitamab vedotin. In some embodiments, the anti-HER2 antibody-drug conjugate comprises an anti-HER2 antibody and a cytotoxic molecule (e.g., wherein one or more cytotoxic molecule(s) is/are linked the anti-HER2 antibody directly or via linker). In some embodiments, the administration of the anti-PD-1 antibody, the antibody-drug conjugate, and the anti-HER2 antibody is a first-line (1L) treatment. In some embodiments, the cancer is HER2-positive.

### Antibody-Drug Conjugates

In some embodiments, the anti-HER2 antibody comprises a heavy chain comprising a heavy chain variable (VH) domain and a light chain comprising a light chain variable (VL) domain. In some embodiments, the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence NYLFDH (SEQ ID NO:3) or ARNYLFDHW (SEQ ID NO: 12); and/or the VL domain comprises a CDR-L1 comprising the amino acid sequence KASQDVGTAVA (SEQ ID NO:4), a CDR-L2 comprising the amino acid sequence WASIRHT (SEQ ID NO:5), and a CDR-L3 comprising the amino acid sequence HQFATYT (SEQ ID NO:6). In some embodiments, the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence NYLFDH (SEQ ID NO:3); and the VL domain comprises a CDR-L1 comprising the amino acid sequence KASQDVGTAVA (SEQ ID NO:4), a CDR-L2 comprising the amino acid sequence WASIRHT (SEQ ID NO:5), and a CDR-L3 comprising the amino acid sequence HQFATYT (SEQ ID NO:6). In some embodiments, the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence ARNYLFDHW (SEQ ID NO:12); and the VL domain comprises a CDR-L1 comprising the amino acid sequence KASQDVGTAVA (SEQ ID NO: 4), a CDR-L2 comprising the amino acid sequence WASIRHT (SEQ ID NO:5), and a CDR-L3 comprising the amino acid sequence HQFATYT (SEQ ID NO:6). In some embodiments, the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence ARNYLFDHW (SEQ ID NO:12); and/or the VL domain comprises a CDR-L1 comprising the amino acid sequence KASQDVGTAVA (SEQ ID NO:4), a CDR-L2 comprising the amino acid sequence WASIRHT (SEQ ID NO:5), and a CDR-L3 comprising the amino acid sequence HQFATYT (SEQ ID NO:6).

In some embodiments, the anti-HER2 antibody comprises a VH domain that comprises the amino acid sequence of SEQ ID NO:7 and/or a VL domain that comprises the amino acid sequence of SEQ ID NO:8. In some embodiments, the anti-HER2 antibody comprises a VH domain that comprises the amino acid sequence of SEQ ID NO:7 and a VL domain that comprises the amino acid sequence of SEQ ID NO:8.

In some embodiments, the anti-HER2 antibody comprises a heavy chain that comprises the amino acid sequence of SEQ ID NO:9 or SEQ ID NO:10 and/or a light chain that comprises the amino acid sequence of SEQ ID NO:11. In some embodiments, the anti-HER2 antibody comprises a heavy chain that comprises the amino acid sequence of SEQ ID NO: 9 or SEQ ID NO:10 and a light chain that comprises the amino acid sequence of SEQ ID NO: 11.

In some embodiments, the anti-HER2 antibody comprises one, two, three, four, five, or six CDR sequences shown in Table 1 below. In some embodiments, the anti-HER2 antibody comprises one, two, or all three CDR sequences from a VH domain or heavy chain sequence shown in Table 1 below and/or one, two, or all three CDR sequences from a VL domain or light chain sequence shown in Table 1 below. In some embodiments, the anti-HER2 antibody comprises a VH domain sequence and/or VL domain sequence shown in Table 1 below. In some embodiments, the anti-HER2 antibody comprises a heavy chain and/or a light chain sequence shown in Table 1 below.

TABLE 1
Anti-HER2 antibody sequences

| Description | Amino acid sequence | SEQ ID NO |
|---|---|---|
| Disitamab | DYYIH | 1 |
| CDR-H1 | | |
| Disitamab | RVNPDHGDSYYNQKFKD | 2 |
| CDR-H2 | | |
| Disitamab | NYLFDH | 3 |
| CDR-H3 | | |
| Disitamab | KASQDVGTAVA | 4 |
| CDR-L1 | | |
| Disitamab | WASIRHT | 5 |
| CDR-L2 | | |
| Disitamab | HQFATYT | 6 |
| CDR-L3 | | |
| Disitamab | EVQLVQSGAEVKKPGATVKISCKVSGYTFTDYYIHW | 7 |
| VH domain | VQQAPGKGLEWMGRVNPDHGDSYYNQKFKDKATIT | |
| | ADKSTDTAYMELSSLRSEDTAVYFCARNYLFDHWG | |
| | QGTLVTVSS | |
| Disitamab | DIQMTQSPSSVSASVGDRVTITCKASQDVGTAVAWY | 8 |
| VL domain | QQKPGKAPKLLIYWASIRHTGVPSRFSGSGSGTDFTL | |
| | TISSLQPEDFATYYCHQFATYTFGGGTKVEIK | |
| Disitamab | EVQLVQSGAEVKKPGATVKISCKVSGYTFTDYYIHW | 9 |
| heavy chain | VQQAPGKGLEWMGRVNPDHGDSYYNQKFKDKATIT | |
| sequence 1 | ADKSTDTAYMELSSLRSEDTAVYFCARNYLFDHWG | |
| | QGTLVTVSSASTKGPSVFPLAPSSKSTSGGTAALGCL | |
| | VKDYFPEPVTVSWNSGALTSGVHTFPAVLQSSGLYS | |
| | LSSVVTVPSSSLGTQTYICNVNHKPSNTKVDKKVEPK | |
| | SCDKTHTCPPCPAPELLGGPSVFLFPPKPKDTLMISRT | |
| | PEVTCVVVDVSHEDPEVKFNWYVDGVEVHNAKTKP | |
| | REEQYNSTYRVVSVLTVLHQDWLNGKEYKCKVSNK | |
| | ALPAPIEKTISKAKGQPREPQVYTLPPSREEMTKNQVS | |
| | LTCLVKGFYPSDIAVEWESNGQPENNYKTTPPVLDSD | |
| | GSFFLYSKLTVDKSRWQQGNVFSCSVMHEALHNHY | |
| | TQKSLSLSPG | |
| Disitamab | EVQLVQSGAEVKKPGATVKISCKVSGYTFTDYYIHW | 10 |
| heavy chain | VQQAPGKGLEWMGRVNPDHGDSYYNQKFKDKATIT | |
| sequence 2 | ADKSTDTAYMELSSLRSEDTAVYFCARNYLFDHWG | |
| | QGTLVTVSSASTKGPSVFPLAPSSKSTSGGTAALGCL | |
| | VKDYFPEPVTVSWNSGALTSGVHTFPAVLQSSGLYS | |
| | LSSVVTVPSSSLGTQTYICNVNHKPSNTKVDKKVEPK | |
| | SCDKTHTCPPCPAPELLGGPSVFLFPPKPKDTLMISRT | |
| | PEVTCVVVDVSHEDPEVKFNWYVDGVEVHNAKTKP | |
| | REEQYNSTYRVVSVLTVLHQDWLNGKEYKCKVSNK | |
| | ALPAPIEKTISKAKGQPREPQVYTLPPSREEMTKNQVS | |
| | LTCLVKGFYPSDIAVEWESNGQPENNYKTTPPVLDSD | |
| | GSFFLYSKLTVDKSRWQQGNVFSCSVMHEALHNHY | |
| | TQKSLSLSPGK | |
| Disitamab | DIQMTQSPSSVSASVGDRVTITCKASQDVGTAVAWY | 11 |
| light chain | QQKPGKAPKLLIYWASIRHTGVPSRFSGSGSGTDFTL | |
| | TISSLQPEDFATYYCHQFATYTFGGGTKVEIKRTVAA | |
| | PSVFIFPPSDEQLKSGTASVVCLLNNFYPREAKVQWK | |
| | VDNALQSGNSQESVTEQDSKDSTYSLSSTLTLSKADY | |
| | EKHKVYACEVTHQGLSSPVTKSFNRGEC | |
| Disitamab | ARNYLFDHW | 12 |
| CDR-H3 | | |
| (long) | | |

In some embodiments, the anti-HER2 antibody is disitamab (RC48). See, e.g., U.S. Pat. No. 10,087,260, the contents of which are incorporated herein by reference in its entirety.

In some embodiments, the cytotoxic molecule of an antibody-drug conjugate of the present disclosure comprises a tubulin inhibitor or DNA damaging agent.

In some embodiments, the tubulin inhibitor comprises a dolastatin or derivative thereof, auristatin or derivative thereof, or maytansinoid or derivative thereof. In some embodiments, the tubulin inhibitor comprises monomethyl auristatin E (MMAE), monomethyl auristatin F (MMAF), or auristatin F (AF). In some embodiments, the tubulin inhibitor comprises mertansine (DM1), maytansine (DM3), or ravtansine (DM4). Structures for MMAE and MMAF are provided below. Additional descriptions and examples of tubulin inhibitors may be found, e.g., in Chen, H. et al. (2017) Molecules 22 (8): 1281.

In some embodiments, the DNA damaging agent comprises a calicheamicin, duocarmycin, pyrrolobenzodiazepine (PBD), or SN-38.

In some embodiments, the cytotoxic molecule comprises an amanitin, anthracycline, baccatin, camptothecin, cemadotin, colchicine, colcemid, combretastatin, cryptophycin, discodermolide, docetaxel, doxorubicin, echinomycin, eleutherobin, epothilone, estramustine, lexitropsin, maytansine, methotrexate, netropsin, puromycin, rhizoxins, taxane, tubulysin, or vinca alkaloid.

In some embodiments, the antibody-drug conjugate is represented by formula Ab-(L-U) n, wherein Ab is the anti-HER2 antibody, L is a linker between the cytotoxic molecule and the anti-HER2 antibody, U is the conjugated cytotoxic molecule, and n is an integer from 1 to 8, representing the number of cytotoxic molecules bound to the antibody. For example, in some embodiments, the antibody-drug conjugate used is named RC48-mc-vc-pAB-MMAE, which conforms to the structure of the general formula Ab-(L-U) n, in which RC48 (a humanized anti-HER2 monoclonal antibody) is coupled to MMAE through the linker mc-vc-pAB, and the number of coupling ranges from 1 to 8, including 1, 2, 3, 4, 5, 6, 7, 8 or a combination of antibody-drug conjugates with varying MMAE coupling numbers ranging from 1 to 8. In some embodiments, the number of cytotoxic molecules bound to the antibody is given as an average number of cytotoxic molecules bound to the antibody, e.g., within a given sample, population, or composition. In some embodiments, an average of 4 MMAE molecules are conjugated to the antibody, e.g., via a linker such as mc-vc-pAB.

In some embodiments, the linker is attached to the anti-HER2 antibody via a thiol or amino moiety. In some embodiments, the cytotoxic molecule is conjugated to the antibody through site-directed or undirected conjugation.

In some embodiments, the linker is selected from the group consisting of maleimidocaproyl valine citrulline p-amino-benzyl (mc-vc-pAB), maleimidocaproyl (mc), triglycyl peptide linker, 3-maleimido-propionic acid, Mal-di-EG-OPFP (perfluorophenyl 3-(2-(2-(3-(2,5-dioxo-2,5-dihydro-1H-pyrrol-1-yl) propanamido) ethoxy) ethoxy) propanoate), Mal-di-EG-Osu (2,5-dioxopyrrolidin-1-yl 3-(2-(2-(2,5-dioxo-2,5-dihydro-1H-pyrrol-1-yl) ethoxy) ethoxy) propanoate), Mal-Tri-EG-Osu (2,5-dioxopyrrolidin-1-yl 3-(2-(2-(2-(2,5-dioxo-2,5-dihydro-1H-pyrrol-1-yl) ethoxy) ethoxy) ethoxy) ethoxypropanoate), Mal-Tetra-EG-Osu (2,5-dioxopyrrolidin-1-yl 1-(2,5-dioxo-2,5-dihydro-1H-pyrrol-1-yl)-3-oxo-7,10,13,16-tetraoxa-4-azanonadecan-19-oate), Br-di-EG-Osu (2,5-dioxopyrrolidin-1-yl 3 (2-(2-(2-bromoacetamido) ethoxy) ethoxy) propanoate), Py-ds-prp-Osu (2-5-dioxopyrrolidin-1-yl 3-(pyridine-2-yldisulfanyl) propanoate), Py-ds-Prp-OPEP (perfluorophenyl 3-(pyridine-2-yldisulfanyl) propanoate), Py-ds-dmBut-Osu (2,5-dioxopyrrolidin-1-yl 4-methyl-4-(pyridine-2-yldisulfanyl) pentanoate, Py-ds-dmBut-OPF (perfluorophenyl 4-methyl-4-(pyridine-2-yldisulfanyl) pentanoate), SMCC (N-succinimidyl 4-(maleimidomethyl) cyclohexanecarboxylate), MBS (3-maleimidobenzoic acid N-hydroxysuccinimide ester), SATA (S-(N-succinimidyl)thioacetate), SPDP ((N-succinimidyl 3-(2-pyridyldithio) propionate), and SMPT ((N-succinimidyloxy carbonyl)-1-methyl-1-(2-pyridyldithio) toluene).

In some embodiments, the linker is a linker described in Table 2 below.

TABLE 2
Exemplary linkers for antibody-drug conjugates.

| Abbreviation | Full name | Feature |
|---|---|---|
| mc | Maleimidocaproyl | Non- |
| | | cleavable |
| mc-vc-pAB | Maleimidocaproyl valine citrulline p-amino-benzyl | Cleavable |
| 3-MPA | 3-maleimido-propionic acid | Non- |
| | | cleavable |
| Mal-di-EG- | Perfluorophenyl | Non- |
| OPFP | 3-(2-(2-(3-(2,5-dioxo-2,5-dihydro-1H-pyrrol-1- | cleavable |
| | yl)propanamido)ethoxy) ethoxy)propanoate | |
| Mal-di-EG-Osu | 2,5-dioxopyrrolidin-1-yl | Non- |
| | 3-(2-(2-(2,5-dioxo-2,5-dihydro-1H-pyrrol-1- | cleavable |
| | yl)ethoxy)ethoxy) propanoate | |
| Mal-Tri-EG-Osu | 2,5-dioxopyrrolidin-1-yl | Non- |
| | 3-(2-(2-(2-(2,5-dioxo-2,5-dihydro-1H-pyrrol-1- | cleavable |
| | yl)ethoxy)ethoxy)ethoxy) propanoate | |
| Mal-Tetra-EG- | 2,5-dioxopyrrolidin-1-yl | Non- |
| Osu | 1-(2,5-dioxo-2,5-dihydro-1H-pyrrol-1-yl)-3-oxo- | cleavable |
| | 7,10,13,16-tetraoxa-4-azanonadecan-19-oate | |
| Br-di-EG-Osu | 2,5-dioxopyrrolidin-1-yl | Non- |
| | 3-(2-(2-(2-bromoacetamido)ethoxy)ethoxy)propanoate | cleavable |
| Py-ds-prp-Osu | 2,5-dioxopyrrolidin-1-yl 3-(32escry32e-2- | Reducible |
| | yldisulfanyl)propanoate | |
| Py-ds-Prp-OPFP | Perfluorophenyl 3-(32escry32e-2- | Reducible |
| | yldisulfanyl)propanoate | |
| Py-ds-dmBut- | 2,5-dioxopyrrolidin-1-yl 4-methyl-4-(32escry32e-2- | Reducible |
| Osu | yldisulfanyl) pentanoate | |
| Py-ds-dmBut- | Perfluorophenyl 4-methyl-4-(32escry32e-2- | Reducible |
| OPF | yldisulfanyl)pentanoate | |
| SMCC | N-succinimidyl 4-(maleimidomethyl) | Non- |
| | cyclohexanecarboxylate | cleavable |
| MBS | 3-maleimidobenzoic acid N-hydroxysuccinimide ester | Non- |
| | | cleavable |
| SATA | S-(N-succinimidyl) thioacetate | Non- |
| | | cleavable |
| SPDP | N-succinimidyl 3-(2-pyridyldithio)propionate | Reducible |
| SMPT | (N-succinimidyl carbonyl)-1-methyl-1-(2- | Reducible |
| | pyridyldithio) toluene | |

In some embodiments, the anti-HER2 antibody-drug conjugate is disitamab vedotin (DV; also referred to as disitamab vedotin). Disitamab vedotin (DV hereafter) is an antibody-drug conjugate (ADC) that targets cancers expressing HER2, an oncogenic growth factor receptor which promotes cell proliferation and survival. DV consists of an anti-HER2 monoclonal antibody disitamab (RC48) conjugated with an average of 4 molecules of the tubulin-disrupting anti-mitotic agent monomethyl auristatin E (MMAE) via a cleavable peptide linker. See, e.g., U.S. Pat. No. 10,087,260 and U.S. PG Pub Nos. 2020/0289663 and 2021/0154314, the contents of which are incorporated herein by reference in their entirety. DV has multimodal antitumor mechanisms of action that include direct cytotoxicity of HER2-expressing cancer cells and bystander effect based-cytotoxicity of neighboring cells, both of which are mediated by the intracellular release of MMAE within the targeted cell. Released MMAE can induce immunogenic cell death (ICD), which promotes immune cell recruitment to the tumor. In addition, DV stimulates Fc-gamma receptor mediated antibody-dependent cellular cytotoxicity (ADCC), which can lead to target cell death. DV also inhibits HER2-activated downstream signaling pathways, further blocking cellular growth and proliferation.

### Combination Therapies

In some embodiments, the method comprises administering to the individual an effective amount of an anti-PD-1 antibody and an anti-human epidermal growth factor receptor 2 (HER2) antibody-drug conjugate. In some embodiments, the method further comprises administering one or more additional agents. In some embodiments, the cancer is gastric cancer or GEJC (e.g., gastroesophageal junction adenocarcinoma), and the method further comprises administering one or more additional agents. In some embodiments, the additional agent is an anti-HER2 antibody (e.g., trastuzumab). In some embodiments, the additional agent is a chemotherapy (e.g., oxalipatin and capecitabine, or CAPOX).

### Anti-PD-1 Antibodies

Provided herein is a method of treating cancer, e.g., gastric cancer or gastroesophageal junction cancer (GEJC), endometrial cancer, or breast cancer, wherein the method comprises administering to the individual an effective amount of an anti-PD-1 antibody and an HER2 antibody-drug conjugate. In some embodiments, the HER2 antibody-drug conjugate is disitamab vedotin.

Programmed death-1 (PD-1) is an inhibitory receptor expressed on T-cells that promotes apoptosis of inflammatory T-cells and inhibits apoptosis of anti-inflammatory regulatory T-cells, thereby promoting self-tolerance and preventing autoimmune diseases. To evade immune surveillance, tumor cells often exploit this system by over-expressing PD-L1, the ligand which binds to and activates PD-1. Anti-PD-1 antibodies are used to block the binding of PD-L1 to PD-1 and thereby enhance immune response against cancer cells. Anti-PD-1 antibodies have showed promising results in various cancers via enhancing T cell functions. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116.

In some embodiments, the anti-PD-1 antibody is a human antibody, a humanized antibody, or a chimeric antibody. In some embodiments, the anti-PD-1 antibody is one or more of toripalimab, MDX-1 106 (nivolumab), MK-3475 (pembrolizumab, e.g., Keytruda®), MEDI-0680 (AMP-514), PDR001, REGN2810, MGA-012, JNJ-63723283, BI 754091, BGB-108, BGB-A317, JS-001, STI-A1110, INCSHR-1210, PF-06801591, TSR-042, AM0001, ENUM 244C8, ENUM 388D4, cemiplimab, or dostarlimab. Other examples of anti-PD-1 antibodies include, but are not limited to, MEDI-0680 (AMP-514; AstraZeneca), PDR001 (CAS Registry No. 1859072-53-9; Novartis), REGN2810 (e.g., LIBTAYO® or cemiplimab-rwlc; Regeneron), BGB-108 (BeiGene), BGB-A317 (BeiGene), BI 754091, JS-001 (Shanghai Junshi), STI-A1110 (Sorrento), INCSHR-1210 (Incyte), PF-06801591 (Pfizer), TSR-042 (also known as ANB011; Tesaro/AnaptysBio), AM0001 (ARMO Biosciences), ENUM 244C8 (Enumeral Biomedical Holdings), or ENUM 388D4 (Enumeral Biomedical Holdings).

In some embodiments, the PD-1 antibody comprises one or more of toripalimab, nivolumab, pembrolizumab, cemiplimab, retifanlimab, or dostarlimab. In some embodiments, the anti-PD-1 antibody is toripalimab.

In some embodiments, the PD-1 antibody comprises one or more of toripalimab, nivolumab, pembrolizumab, cemiplimab, retifanlimab, or dostarlimab.

Toripalimab (INN 10820), also known as JS-001, TAB-001, teriprolizumab, Loqtorzi, and Tuoyi, refers to an IgG4 selective, recombinant, humanized monoclonal antibody against programmed death protein 1 (PD-1), which is able to bind to PD-1 and block the interaction with its ligands. Toripalimab is the first monoclonal anti-PD-1 antibody approved by the China NMPA onto the market. It consists of two heavy chains of 452 amino acids, two light chains of 219 amino acids, and includes an N-linked glycosylation site at N302 in each heavy chain. The average molecular weight of toripalimab is 149,670 Daltons. The interaction of toripalimab with PD-1 is mainly attributed to the complementarity determining regions of the heavy chain of the former and the FG loop of the latter; the light chain complementarity determining regions of toripalimab participate mainly in recognizing the epitopes on PD-1. As used herein, “toripalimab” may refer to toripalimab, an antibody comprising the same heavy and/or light chain sequences as toripalimab, or an antibody comprising the same VL and/or VL sequences as toripalimab. In some embodiments, the PD-1 antibody comprises one, two, three, four, five, or six CDR sequences shown in Table 3 below. In some embodiments, the PD-1 antibody comprises one, two, or all three CDR sequences from a VH domain or heavy chain sequence shown in Table 3 below and/or one, two, or all three CDR sequences from a VL domain or light chain sequence shown in Table 3 below. In some embodiments, the PD-1 antibody comprises a VH domain sequence and/or VL domain sequence shown in Table 3 below. In some embodiments, the PD-1 antibody comprises a heavy chain and/or a light chain sequence shown in Table 3 below.

In some embodiments, toripalimab is administered in a pharmaceutical composition comprising toripalimab and one or more pharmaceutically acceptable carriers. The pharmaceutical compositions are typically administered as an intravenous infusion. In some embodiments, toripalimab is administered intravenously.

TABLE 3
Anti-PD-1 antibody sequences

| Description | Amino acid sequence | SEQ ID NO |
|---|---|---|
| Toripalimab | DYEMH | 13 |
| CDR-H1 | | |
| Toripalimab | VIESETGGTAYNQKFKG | 14 |
| CDR-H2 | | |
| Toripalimab | EGITTVATTYYWYFDV | 15 |
| CDR-H3 | | |
| Toripalimab | RSSQSIVHSNGNTYLE | 16 |
| CDR-L1 | | |
| Toripalimab | KVSNRFS | 17 |
| CDR-L2 | | |
| Toripalimab | FQGSHVPLT | 18 |
| CDR-L3 | | |
| Toripalimab | QGQLVQSGAE VKKPGASVKV SCKASGYTFT | 19 |
| VH domain | DYEMHWVRQA PIHGLEWIGV | |
| | IESETGGTAY NQKFKGRVTI TADKSTSTAY | |
| | MELSSLRSED TAVYYCAREG | |
| | ITTVATTYYW YFDVWGQGTT VTVSS | |
| Toripalimab | DVVMTQSPLS LPVTLGQPAS ISCRSSQSIV | 20 |
| VL domain | HSNGNTYLEW YLQKPGQSPQ | |
| | LLIYKVSNRF SGVPDRFSGS GSGTDFTLKI | |
| | SRVEAEDVGV YYCFQGSHVP | |
| | LTFGQGTKLE IKRTV | |
| Toripalimab | QGQLVQSGAE VKKPGASVKV SCKASGYTFT | 21 |
| heavy chain | DYEMHWVRQA PIHGLEWIGV | |
| | IESETGGTAY NQKFKGRVTI TADKSTSTAY | |
| | MELSSLRSED TAVYYCAREG | |
| | ITTVATTYYW YFDVWGQGTT VTVSSASTKG | |
| | PSVFPLAPCS RSTSESTAAL | |
| | GCLVKDYFPE PVTVSWNSGA LTSGVHTFPA | |
| | VLQSSGLYSL SSVVTVPSSS | |
| | LGTKTYTCNV DHKPSNTKVD KRVESKYGPP | |
| | CPPCPAPEFL GGPSVFLFPP | |
| | KPKDTLMISR TPEVTCVVVD VSQEDPEVQF | |
| | NWYVDGVEVH NAKTKPREEQ | |
| | FNSTYRVVSV LTVLHQDWLN GKEYKCKVSN | |
| | KGLPSSIEKT ISKAKGQPRE | |
| | PQVYTLPPSQ EEMTKNQVSL TCLVKGFYPS | |
| | DIAVEWESNG QPENNYKTTP | |
| | PVLDSDGSFF LYSRLTVDKS RWQEGNVFSC | |
| | SVMHEALHNH YTQKSLSLSL | |
| | GK | |
| Toripalimab | DVVMTQSPLS LPVTLGQPAS ISCRSSQSIV | 22 |
| light chain | HSNGNTYLEW YLQKPGQSPQ | |
| | LLIYKVSNRF SGVPDRFSGS GSGTDFTLKI | |
| | SRVEAEDVGV YYCFQGSHVP | |
| | LTFGQGTKLE IKRTVAAPSV FIFPPSDEQL | |
| | KSGTASVVCL LNNFYPREAK | |
| | VQWKVDNALQ SGNSQESVTE QDSKDSTYSL | |
| | SSTLTLSKAD YEKHKVYACE | |
| | VTHQGLSSPV TKSFNRGEC | |

### Chemotherapies

Provided herein is a method of treating cancer, e.g., gastric cancer or gastroesophageal junction cancer (GEJC), e.g., gastroesophageal junction adenocarcinoma, wherein the method comprises administering to the individual an effective amount of an anti-PD-1 antibody and an HER2 antibody-drug conjugate and a chemotherapy. In some embodiments, the anti-PD-1 antibody is toripalimab. In some embodiments, the HER2 antibody-drug conjugate is disitamab vedotin. In some embodiments, the cancer is HER2-positive, HER2-low, or HER2-zero.

In some embodiments, the chemotherapy is oxaliplatin and capecitabine (CAPOX). The combination of oxaliplatin and capecitabine is also known as XELOX, CAPE-OX, or OxCap.

Oxaliplatin is an antineoplastic agent. In some embodiments, oxaliplatin is formulated as a solution. In some embodiments, oxaliplatin is formulated as a powder for solution. Oxaliplatin is also known as Eloxatin. Oxaliplatin has the chemical formula C8H14N204Pt, and a molecular mass of 397.294 g·mol-1. In some embodiments, oxaliplatin is administered intravenously.

Capecitabine is an antineoplastic agent. In some embodiments, oxaliplatin is a solution. In some embodiments, oxaliplatin is a powder for solution. Capecitabine is also known as Xeloda, Xitabin, or Kapetral. Oxaliplatin has the chemical formula C15H22FN306, and a molecular mass of 359.354 g· mol-1. In some embodiments, capecitabine is formulated as a tablet. In some embodiments, capecitabine is administered orally.

In some embodiments, CAPOX is administered in a pharmaceutical composition comprising CAPOX and one or more pharmaceutically acceptable carriers. In some embodiments, oxaliplatin is administered intravenously. In some embodiments, capecitabine is administered orally.

### Anti-HER2 Antibodies

Provided herein is a method of treating cancer, e.g., gastric cancer or gastroesophageal junction cancer (GEJC), e.g., gastroesophageal junction adenocarcinoma, wherein the method comprises administering to the individual an effective amount of an anti-PD-1 antibody and an HER2 antibody-drug conjugate and an anti-HER2 antibody. In some embodiments, the anti-PD-1 antibody is toripalimab. In some embodiments, the HER2 antibody-drug conjugate is disitamab vedotin. In some embodiments, the cancer is HER2-positive.

In some embodiments, the anti-HER2 antibody is trastuzumab. As used herein, “trastuzumab” may refer to trastuzumab, an antibody comprising the same heavy and/or light chain sequences as trastuzumab, or an antibody comprising the same VL and/or VL sequences as trastuzumab. In some embodiments, the anti-HER2 antibody comprises the same six CDR sequences as trastuzumab. In some embodiments, the anti-HER2 antibody comprises the same VH domain sequence and/or VL domain sequence as trastuzumab. In some embodiments, the anti-HER2 antibody comprises the same heavy chain and/or a light chain sequence as trastuzumab.

Trastuzumab is also known as Herceptin. Trastuzumab is a humanized monoclonal antibody that binds to HER2 receptors present on the surface of HER2-positive tumor cells, blocking them from receiving growth signals and flagging them for destruction by the immune system. The average molecular weight of trastuzumab is 149,670 Daltons. In some embodiments, trastuzumab is formulated as a lyophilized powder. In some embodiments, trastuzumab is formulated as a solution for injection. In some embodiments, trastuzumab is administered intravenously.

### Cancers

In some embodiments, the cancer is gastric cancer, gastroesophageal junction cancer (GEJC), breast cancer, endometrial cancer. In some embodiments, the cancer is gastric cancer. In some embodiments, the cancer is gastric adenocarcinoma or gastroesophageal junction adenocarcinoma. In some embodiments, the cancer is endometrial cancer. In some embodiments, the cancer is breast cancer. In some embodiments, the cancer is locally advanced or metastatic. In some embodiments, the cancer is locally advanced or metastatic gastric cancer or GEJC (LA/mGC/GEJC). In some embodiments, the cancer is locally advanced or metastatic endometrial cancer (LA/mEC). In some embodiments, the cancer is a HER2-positive/HER2-low, locally advanced or metastatic gastric cancer. In some embodiments, the cancer is a HER2-positive/HER2-low locally advanced or metastatic gastroesophageal junction cancer. In some embodiments, the cancer is locally advanced or metastatic endometrial cancer (LA/mEC). In some embodiments, the cancer is locally advanced or metastatic breast cancer (LA/mBC). In some embodiments, the cancer is a hormone receptor (HR)-negative, HER2-low expressing, unresectable locally advanced or metastatic breast cancer. In some embodiments, the cancer is a standard therapy progressed HR-positive, HER2-low expressing, unresectable locally advanced or metastatic breast cancer. In some embodiments, the cancer is a HR+HER2-low expressing breast cancer wherein the individual had received at least one line of chemotherapy. In some embodiments, the cancer is a HR-HER2-low expressing breast cancer wherein the individual had received at least one line of chemotherapy.

In some embodiments, cells of the cancer express HER2. In some embodiments, the cancer is a HER2-positive (HER2+) cancer. In some embodiments, cells of the cancer exhibit HER2 gene amplification. In some embodiments, cells of the cancer overexpress HER2, e.g., on their cell surface. Overexpression and/or amplification of HER2 is seen in many malignancies including breast, gastric, ovarian, pancreatic, colorectal, and endometrial cancer (Neve, R. M. et al. (2001) Ann Oncol 12 (Suppl1): S9-S13; Menard, S. et al. (2003) Oncogene 22 (42): 6570-6578; Moasser, M. M. (2007) Oncogene 26 (45): 6469-6487; Iqbal, N. et al. (2014) Mol Biol Int 2014:852748). In some embodiments, cells of the cancer express HER2, wherein the cells have an IHC score of IHC≥1 and/or are ISH positive.

In some embodiments, a sample obtained from the individual comprises cancer cells that express HER2 on their cell surface, as measured by an immunohistochemistry (IHC) assay. IHC assays for HER2 involve semi-quantitative measurements of HER2 cellular expression/overexpression. These are typically done by staining a sample including cancer cells using a primary anti-HER2 antibody, followed by visualization using a labeled secondary antibody and subsequent analysis of HER2 expression, e.g., membrane expression on the surface. Variables factoring into the IHC score (given on a scale from 0 to 3+) can include intensity of staining, location and/or completeness of staining (e.g., surface/membrane expression), and proportion of tumor cells exhibiting staining. Methods and criteria for determining HER2 status by IHC are known in the art and can be found, e.g., in Wolff, A. C. et al. (2013) J Clin Oncol 31 (31): 3997-4013 and Wolff, A. C. et al. (2018) J Clin Oncol 36 (20): 2105-2122. See also the HercepTest™ semi-quantitative IHC test kit.

Evaluation of HER2 by IHC may involve one or more, or all, of the steps of:
1. The entire section is first observed under low magnification to determine whether the staining is satisfactory and whether there is heterogeneity in HER2 expression;
2. Quality control slides are read when evaluating; cytoplasmic and nuclear staining should be negligible, and normal epithelium should not show strong cell membrane staining;
3. Tissue margins and poorly prepared (e.g., obviously extruded) cancer tissue is ignored during evaluation.
4. If the tumor has obvious heterogeneity, the percentage of each scoring level is indicated separately when interpreting.
5. If invasive cancer is the object during evaluation, it is indicated separately if the non-invasive cancer part has overexpressed HER2 (2+ or 3+).
6. If multiple blocks or sections are detected, results are reported separately.

In some embodiments, a sample obtained from the individual comprises cancer cells that exhibit HER2 gene amplification, as measured by an in situ hybridization (ISH) assay (ISH-positive). In some embodiments, a sample obtained from the individual comprises cancer cells that do not exhibit HER2 gene amplification, as measured by an in situ hybridization (ISH) assay (ISH-negative). ISH assays for HER2 gene amplification typically involve measurement of level of hybridization to a HER2-specific probe using microscopy. In some embodiments, cells are stained with dual probes: a HER2-specific probe, and a control probe (hybridizing to, e.g., chromosome 17 or CEP17), such that the ratio of HER2: control signal is indicative of HER2 copy number and/or amplification. Variables factoring into the ISH status can include number of signal copies of HER2, ratio of HER2: control copy number, and formation of HER2 clusters. Methods and criteria for determining HER2 amplification status by ISH are known in the art and can be found, e.g., in Wolff, A. C. et al. (2013) J Clin Oncol 31 (31): 3997-4013 and Wolff, A. C. et al. (2018) J Clin Oncol 36 (20): 2105-2122. Detection of HER2 may be performed by FISH, e.g., using one or more, or all, of the following steps:

(1) Selecting a representative wax block of tumor tissue. Section by professional and technical personnel, the section is complete, smooth, of uniform thickness, without affecting the diagnosis of knife mark wrinkles. (Tissue containing calcified particles and other uncontrollable factors are excluded), section thickness: 4-5 μm;

(2) Tissue section pretreatment using either of the following methods:

### Method 1 (Manual Operation):

a) Immerse in xylene and dewaxed twice, 15 minutes each time, and then immerse in 100% ethanol for 5 minutes at room temperature,
b) Rehydrate in 100% ethanol, 85% ethanol and 70% ethanol for 2 minutes respectively at room temperature, then immerse in deionized water at room temperature for 3 minutes,
c) Treatment with 90˜93° C. deionized water for 20 minutes,
d) 1 ml gastric enzyme storage solution (200 mg/mL) is dissolved in 200 ml 0.01 MHCL to obtain gastric enzyme working solution (1 mg/ml); Soak the tissue section in gastric enzyme working solution and incubate at 37° C. for 15-30 minutes (the time depends on the thickness of the tissue, generally about 20 minutes),
e) After digestion by gastric enzymes, then rinse in deionized water for 5 minutes,
f) Dehydrate respectively in 70% ethanol, 85% ethanol and 100% ethanol for 2 minutes at room temperature,
g) After drying, then perform the following hybrid denaturation.

### Method 1 (Fully Automatic):

a) Soak in xylene for dewaxing twice at room temperature for 15 minutes each, and then immerse in 100% ethanol twice for 5 minutes each,
b) Dry tissue section at room temperature,
c) Initialize the system and select program, fill the reagent according to the instrument algorithm,
d) Place the dry slides tissue face upward on the glass shelf, put it in the reaction tank, cover the reaction tank cover, close the machine cover, and run the selected program;

(3) Hybridization apparatus denatured hybridization using the following steps:
a) Drop 10 μL probe mixture into the slide hybridization area, immediately cover the slide and seal the edge with rubber glue,
b) Prepare hybridization machine, covariance condition: 75° C., 5 minutes, hybridization condition: 37° C., 16 h; (be careful to maintain humidity in hybridization instrument);

(4) Glass slide rinsing (need to avoid light operation) using the following steps:
a) Carefully remove the cover glass slide, place the glass slide in a solution of 0.3% NP-40/2×SSC at 73° C., shake for 1˜3 seconds, wash for 2 minutes,
b) Rinse at room temperature in 70% ethanol for 3 minutes;

(5) Counterstaining using the following steps:
a) Naturally dried glass slides in dark;
b) Drop 10 μL DAPI at the hybridization site and immediately cover the cover glass. Put in the dark for 10 to 20 minutes, then observe the glass slides under fluorescence microscope with appropriate filter group.

Assessment of HER2, e.g., in a FISH section, for example, generated as described above, may be performed using any suitable method known in the art. For example, using one or more, or all of the following steps:
(a) Observe whole FISH section under low magnification to preliminarily determine the test quality (such as the normal cell signals of normal tissues in the specimen) and whether there is heterogeneity in HER2 amplification;
(b) Find at least 2 areas of invasive cancer and count at least 20 invasive cancer cells. FISH is not suitable for microinvasive nidus with too few cells;
(c) IHC sections can be used to determine the areas of invasive cancer that may be amplified; and
(d) Observe HER2 and CEP17 signals through a specific channel filter under high magnification (60× or 100× objective), and calculate the signal count and ratio.

In some embodiments, the sample is a biopsy sample, e.g., from a core needle biopsy. In some embodiments, the sample is from an incisional or excisional surgical procedure. In some embodiments, the sample is a formalin-fixed paraffin embedded (FFPE) tissue block or sample, e.g., with corresponding H&E stain. In some embodiments, the sample comprises unstained slides sectioned from a tissue block, e.g., FFPE tissue block. In some embodiments, the sample is from a primary tumor or metastasis, e.g., from the chest wall, regional lymph node, or a distant organ. In some embodiments, the same sample is used for IHC and ISH assays. In some embodiments, different samples from the same individual are used for IHC and ISH assays.

In some embodiments, cells of the cancer express HER2. In some embodiments, cells of the cancer express HER2, wherein the cells have an IHC score of IHC≥1 (e.g., IHC1+, IHC2+, IHC3+) and/or are ISH-positive.

In some embodiments, the cancer is a HER2-low cancer. In some embodiments, a sample obtained from the individual (e.g., from the cancer of the individual) comprises cancer cells that express HER2 on their cell surface at a level of IHC1+, as measured by IHC assay (IHC1+). In some embodiments, a sample obtained from the individual (e.g., from the cancer of the individual) comprises cancer cells that express HER2 on their cell surface at a level of IHC2+, as measured by IHC assay (IHC2+); and the sample obtained from the individual comprises cancer cells that do not exhibit HER2 gene amplification, as measured by ISH assay (IHC2+/ISH-negative). In some embodiments, a sample obtained from the individual (e.g., from the cancer of the individual) is IHC1+. In some embodiments, a sample obtained from the individual (e.g., from the cancer of the individual) is IHC2+/ISH-negative.

In some embodiments, the cancer is a HER2-positive cancer. In some embodiments, a sample obtained from the individual (e.g., from the cancer of the individual) comprises cancer cells that express HER2 on their cell surface at a level of IHC3+, as measured by IHC assay (IHC3+). In some embodiments, a sample obtained from the individual (e.g., from the cancer of the individual) comprises cancer cells that express HER2 on their cell surface at a level of IHC2+, as measured by IHC assay (IHC2+); and wherein the sample obtained from the individual (e.g., from the cancer of the individual) comprises cancer cells that exhibit HER2 gene amplification, as measured by ISH assay (IHC2+/ISH-positive). In some embodiments, a sample obtained from the individual (e.g., from the cancer of the individual) is IHC3+. In some embodiments, a sample obtained from the individual (e.g., from the cancer of the individual) is IHC3+/ISH+.

In some embodiments, the cancer is HER2-positive LA/mEC. In some embodiments, the cancer is HER2-low LA/mEC. In some embodiments, the cancer is HER2-positive LA/mGC/GEJC. In some embodiments, the cancer is HER2-low LA/mGC/GEJC. In some embodiments, the cancer is HER2-positive LA/mBC. In some embodiments, the cancer is HER2-low LA/mBC.

In some embodiments, the cancer is HER2-low (e.g., IHC2+/ISH-negative or IHC1+) second-line advanced gastric or gastroesophageal junction adenocarcinoma. In some embodiments, the cancer is HER2+ (e.g., IHC2+/ISH+ or IHC3+) third-line or higher advanced gastric or gastroesophageal junction adenocarcinoma. In some embodiments, the cancer is HER2-low (e.g., IHC2+/ISH-negative or IHC1+) second- or third-line advanced gastric or gastroesophageal junction adenocarcinoma. In some embodiments, the cancer is HER2-low LA/mGC/GEJC. In some embodiments, the cancer is HER2+LA/mBC. In some embodiments, the cancer is HER2-low LA/mBC.

In some embodiments, the cancer expresses a low level of HER2, but is classified as a HER2-zero (HER2-0) cancer. In some embodiments, a sample obtained from the individual (e.g., from the cancer of the individual) comprises cancer cells that express HER2 on their cell surface at a level of IHC 0, as measured by IHC assay (IHC 0). In some embodiments, a sample obtained from the individual (e.g., from the cancer of the individual) is IHC 0. In some embodiments, the cancer is HER2-zero (IHC 0) gastric cancer or gastroesophageal junction cancer (GEJC). In some embodiments, the cancer is HER2-zero, and HER2 is not expressed. In some embodiments, the cancer is HER2-zero (IHC 0) LA/m gastric cancer. In some embodiments, the cancer is HER2-zero (IHC 0) LA/m gastroesophageal junction cancer (GEJC).

In some embodiments, the cancer is hormone receptor (HR)-negative (HR−) breast cancer. In some embodiments, the breast cancer is a HR-negative, HER2-low expressing, unresectable locally advanced or metastatic breast cancer. In some embodiments, the cancer is a HR-HER2-low expressing breast cancer wherein the individual had received at least one line of chemotherapy.

In some embodiments, the cancer is HR-positive (HR+) breast cancer. In some embodiments, the breast cancer is a HER2-low expressing, unresectable locally advanced or metastatic breast cancer. In some embodiments, the cancer is a HR+HER2-low expressing breast cancer wherein the individual had received at least one line of chemotherapy.

In some embodiments, the cancer is gastric or gastroesophageal junction cancer, e.g., locally advanced or metastatic gastric cancer or locally advanced or metastatic gastroesophageal junction cancer. In some embodiments, the administration of an anti-PD-1 antibody and the antibody-drug conjugate according to the methods of the present disclosure is a second-line (2L) treatment. In some embodiments, the individual has gastric cancer or gastroesophageal junction cancer, e.g., HER2+ (e.g., IHC2+/ISH+ or IHC3+) third-line or higher advanced gastric or gastroesophageal junction cancer. In some embodiments, the cancer is endometrial cancer, e.g., locally advanced or metastatic endometrial cancer (LA/mEC). In some embodiments, the individual has endometrial cancer, e.g., HER2+ (e.g., IHC2+/ISH+ or IHC3+) second-line advanced endometrial cancer. In some embodiments, the cancer is breast cancer, e.g., locally advanced or metastatic breast cancer (LA/mBC). In some embodiments, the individual has breast cancer, e.g., HER2+ (e.g., IHC2+/ISH+ or IHC3+) second-line (2L) advanced breast cancer.

In some embodiments, the cancer is gastric or gastroesophageal junction cancer, e.g., locally advanced or metastatic gastric cancer or locally advanced or metastatic gastroesophageal junction cancer. In some embodiments, the administration of an anti-PD-1 antibody and the antibody-drug conjugate according to the methods of the present disclosure is a third-line or higher (3L+) treatment. In some embodiments, the individual has gastric cancer or gastroesophageal junction cancer, e.g., HER2+ (e.g., IHC2+/ISH+ or IHC3+) third-line or higher advanced gastric or gastroesophageal junction cancer. In some embodiments, the cancer is endometrial cancer, e.g., locally advanced or metastatic endometrial cancer (LA/mEC). In some embodiments, the administration of an anti-PD-1 antibody and the antibody-drug conjugate according to the methods of the present disclosure is a third-line or higher (3L+) treatment. In some embodiments, the individual has endometrial cancer, e.g., HER2+ (e.g., IHC2+/ISH+ or IHC3+) third-line or higher advanced endometrial cancer. In some embodiments, the cancer is breast cancer, e.g., locally advanced or metastatic breast cancer (LA/mBC). In some embodiments, the administration of an anti-PD-1 antibody and the antibody-drug conjugate according to the methods of the present disclosure is a third-line or higher (3L+) treatment. In some embodiments, the individual has breast cancer, e.g., HER2+ (e.g., IHC2+/ISH+ or IHC3+) third-line or higher advanced breast cancer.

In some embodiments, the cancer is gastric or gastroesophageal junction cancer (e.g., gastroesophageal junction adenocarcinoma), e.g., locally advanced or metastatic gastric cancer or locally advanced or metastatic gastroesophageal junction cancer. In some embodiments, the administration of an anti-PD-1 antibody, the antibody-drug conjugate, and a chemotherapy according to the methods of the present disclosure is a first-line treatment. In some embodiments, the administration of an anti-PD-1 antibody, the antibody-drug conjugate, and an anti-HER2 antibody according to the methods of the present disclosure is a first-line treatment. In some embodiments, the individual has HER2+ (e.g., IHC2+/ISH+ or IHC3+) gastric cancer or gastroesophageal junction cancer, e.g., HER2+ (e.g., IHC2+/ISH+ or IHC3+), and the administration of an anti-PD-1 antibody, the antibody-drug conjugate, and a chemotherapy according to the methods of the present disclosure is a first-line treatment. In some embodiments, the individual has HER2+ (e.g., IHC2+/ISH+ or IHC3+) gastric cancer or gastroesophageal junction cancer, e.g., HER2+ (e.g., IHC2+/ISH+ or IHC3+), and the administration of an anti-PD-1 antibody, the antibody-drug conjugate, and an anti-HER2 antibody according to the methods of the present disclosure is a first-line treatment.

### Individuals to be Treated

In some embodiments, e.g., prior to treatment according to the methods disclosed herein, the individual has been previously treated for the cancer. In some embodiments, the cancer has previously been treated. For example, in some embodiments, the administration of anti-PD-1 antibody and the antibody-drug conjugate according to the methods of the present disclosure is a second-line (2L) treatment or a third-line or higher (3L+) treatment.

In some embodiments, e.g., prior to treatment according to the methods disclosed herein, the individual has experienced progression on or after one or more standard of care therapies. In some embodiments, the individual is intolerant to one or more standard of care therapies. In some embodiments, the individual is unable to receive treatment. In some embodiments, the standard of care is unavailable.

In some embodiments, the individual has a Combined Positive Score (CPS) ≥1. CPS evaluates the number of PD-L1-staining cells (tumor cells, lymphocytes, macrophages) relative to all viable tumor cells. The Combined Positive Score (CPS) is determined by dividing the number of PD-L1 cells (tumor cells, lymphocytes, macrophages) by the total number of viable tumor cells (PD-L1-positive and PD-L1-negative tumor cells), multiplied by 100. The individual or sample from the individual are considered to have PD-L1 expression if CPS ≥1.

In some embodiments, e.g., prior to treatment according to the methods disclosed herein, the individual is at least 18 years old. In some embodiments, e.g., prior to treatment according to the methods disclosed herein, the individual is diagnosed histologically or cytologically with metastatic or locally advanced unresectable HER2-expressing solid tumors. In some embodiments, e.g., prior to treatment according to the methods disclosed herein, the individual has failed first-line or standard treatment (progressive disease or intolerable toxic side effects after treatment) or has no effective treatment methods. In some embodiments, e.g., prior to treatment according to the methods disclosed herein, the individual has at least 1 measurable lesion at baseline according to RECIST 1.1 criteria. In some embodiments, e.g., prior to treatment according to the methods disclosed herein, the individual has only one measurable lesion at baseline, and the lesion area has not received radiation therapy in the past, or there is evidence of significant progression after radiation therapy. In some embodiments, e.g., prior to treatment according to the methods disclosed herein, the individual has an ECOG performance status score of 0 or 1. In some embodiments, one or more of the following apply for the individual receiving treatment according to the methods disclosed herein:
LVEF≥50%, measured by Echocardiogram (ECHO), only measured by cardiac Multigated Radionuclide Angiography (MUGA) when there is no ECHO. Baseline and follow-up measurements remain consistent;
NYHA cardiac function grading<3;
Liver function within 7 days prior to first dose meets the following criteria:
   Total bilirubin≤1.5×ULN (total bilirubin≤3×ULN in subjects with Gilbert's syndrome or liver metastasis);
   Transaminase (ALT/AST)≤2.5×ULN (liver metastasis subjects≤5×ULN);
Renal function within 7 days before first dose: serum muscle ff<1.0×ULN and serum creatinine clearance rate_60 mL/min (calculated according to Cockcroft-Gault formula);
Bone marrow function within 7 days prior to first dose meets the following criteria:
   Hemoglobin≥9.0 g/dL;
   Absolute neutrophil count≥1.5×109/L;
   Platelets count≥100×109/L;
   International Normalized ratio (INR)≤1.5 or partially activated prothrombin time (APTT)≤1.5×ULN

In some embodiments, e.g., prior to treatment according to the methods disclosed herein, the individual has received blood transfusion treatment, the test results after 28 days of transfusion must meet the above criteria. In some embodiments, the expected survival of the individual is at least 3 months.

In some embodiments, e.g., prior to treatment according to the methods disclosed herein, the individual does not have untreated active brain metastasis or meningeal metastasis. In some embodiments, e.g., prior to treatment according to the methods disclosed herein, the individual has brain metastasis, and the brain metastasis has been treated and the condition of the metastasis is stable (brain imaging examination at least 4 weeks before the first dose shows that the lesion is stable and there are no new neurological symptoms, or the neurological symptoms have returned to baseline level), and there is no evidence of new or original brain metastasis enlargement. In some embodiments, e.g., prior to treatment according to the methods disclosed herein, the individual has not received other anti-tumor treatments within 5 half-lives or 2 weeks prior to the first dose (chemotherapy, targeted therapy, biological immunotherapy, traditional Chinese medicine with clear anti-tumor ingredients in the instructions). In some embodiments, e.g., prior to treatment according to the methods disclosed herein, the individual has not received major surgery within 28 days prior to the first dose (such as abdominal, thoracic and other major surgery; excluding diagnostic puncture or peripheral vascular pathway replacement). In some embodiments, e.g., prior to treatment according to the methods disclosed herein, the individual previously received immune checkpoint blockers, T cell co-stimulatory drugs or ADC drugs, including but not limited to PD-1, PD-L1, CTLA4, LAG3 and other immune checkpoint blockers, therapeutic vaccines, T-DM1, etc., excluding non-squamous non-small cell lung cancer and biliary tumors previously treated with PD-1/PD-L1 (excluding toripalimab). In some embodiments, the individual does not require systemic corticosteroids (>10 mg/day of prednisone, or equivalent of other corticosteroids) or immunosuppressant therapy within 14 days before the first dose (excluding inhalation or topical use of hormones, or receiving physiological replacement doses of hormone therapy due to adrenal insufficiency). In some embodiments, e.g., prior to treatment according to the methods disclosed herein, the individual does not have a history of any active autoimmune disease or autoimmune disease, including but not limited to interstitial pneumonia, uveitis, inflammatory bowel disease, hepatitis, pituitary inflammation, vasculitis, systemic lupus erythematosus, etc. (excluding leukorrhea and psoriasis that do not need systematic treatment in recent 2 years, hypothyroidism that can be controlled only by hormone replacement therapy, and type I diabetes patients who only need insulin replacement therapy). In some embodiments, e.g., prior to treatment according to the methods disclosed herein, the individual is not suffering from uncontrolled comorbidities, including but not limited to the following:
Active HBV or HCV infection;
History of HIV infection or AIDS;
Active Tuberculosis;
Active infection or unexplained fever (body temperature≥38.5° C.) occurring during screening and before first dose;
Suffering from systemic diseases without stable control, including diabetes, hypertension, pulmonary fibrosis, acute lung disease, interstitial lung disease, liver cirrhosis, angina pectoris, severe arrhythmia, etc.

In some embodiments, e.g., prior to treatment according to the methods disclosed herein, the individual does not have a prior history of interstitial pneumonia requiring steroid treatment, or are currently in the stage of interstitial pneumonia, or are suspected of having interstitial pneumonia. In some embodiments, the individual does not have a history of allogeneic bone marrow or organ transplantation. In some embodiments, e.g., prior to treatment according to the methods disclosed herein, the individual does not have hypersensitivity or delayed anaphylaxis to certain components or similar drugs of disitamab vedotin and toripalimab injection.

In some embodiments, e.g., prior to treatment according to the methods disclosed herein, the individual has not been previously treated for the cancer. In some embodiments, the cancer has not previously been treated. In some embodiments, the administration of an anti-PD-1 antibody, a chemotherapy, and the antibody-drug conjugate according to the methods of the present disclosure is a first (1L) treatment. In some embodiments, the administration of an anti-PD-1 antibody, an anti-HER2 antibody, and the antibody-drug conjugate according to the methods of the present disclosure is a first (1L) treatment.

In some embodiments, e.g., prior to treatment according to the methods disclosed herein, the individual has gastric cancer or gastroesophageal junction cancer (GEJC), and one or more of the following apply for the individual receiving treatment according to the methods disclosed herein:
1. Bone marrow function:
   Hemoglobin ≥9 g/dL;
   Absolute neutrophil count (ANC) ≥1.5×10⁹/L;
   Platelet ≥100×10⁹/L;
2. Liver function (based on the normal value of the clinical trial center):
   Serum total bilirubin≤1.5 times the upper limit of normal (ULN);
   Without liver metastasis: ALT and AST≤2.5×ULN, with liver metastases: ALT and AST≤5×ULN;
3. Renal function (based on the normal value of the clinical trial center):
   Serum creatinine≤1.5×ULN or creatinine clearance (CrCl) ≥50 mL/min according to Cockcroft-Gault formula or 24-hour urine CrCl≥60 rmL/min;
4. Coagulation function: International Normalized ratio (INR), activated partial thromboplastin time (APTT) and prothrombin time (PT) are all≤1.5 times ULN;
5. Heart function:
   NYHA cardiac function grading<3;
   Left ventricular ejection fraction ≥50%;
   Screening period QTcF interval≤470 ms (based on the average of 3 screening electrocardiograms).

In some embodiments, e.g., prior to treatment according to the methods disclosed herein, the individual has gastric cancer or gastroesophageal junction cancer (GEJC), and one or more of the following apply for the individual receiving treatment has not previously received systemic chemotherapy for locally advanced or metastatic gastric cancer. In some embodiments, the GEJC is gastroesophageal junction cancer. In some embodiments, e.g., prior to treatment according to the methods disclosed herein, the individual has histologically and/or cytologically confirmed locally advanced or metastatic gastric cancer that is inoperable. In some embodiments, e.g., prior to treatment according to the methods disclosed herein, the individual does not have progressive disease/recurrence for more than 6 months after the end of neoadjuvant/adjuvant therapy. In some embodiments, e.g., prior to treatment according to the methods disclosed herein, the individual has confirmed expression status of PD-L1 (CPS≥1 or CPS <1). In some embodiments, e.g., prior to treatment according to the methods disclosed herein, the individual has confirmed expression status of HER2: IHC 1+, 2+ (to determine FISH status) or 3+ or IHC 0. In some embodiments, e.g., prior to treatment according to the methods disclosed herein, the individual has at least one measurable lesion according to RECISTv1.1 standard.

### Administration

In some embodiments, the antibody-drug conjugate is administered to the individual at a dose of 1.5 mg/kg, 2.0 mg/kg or 2.5 mg/kg. In some embodiments, the antibody-drug conjugate is administered to the individual at a dose of 2.0 mg/kg or 2.5 mg/kg. In some embodiments, the antibody-drug conjugate is administered to the individual at a dose of 1.5 mg/kg. In some embodiments, the antibody-drug conjugate is administered to the individual at a dose of 2.0 mg/kg. In some embodiments, the antibody-drug conjugate is administered to the individual at a dose of 2.5 mg/kg. In some embodiments, the antibody-drug conjugate is administered intravenously to the individual.

In some embodiments, the antibody-drug conjugate is administered to the individual at a dose of 1.5 mg/kg to 2.5 mg/kg. In some embodiments, the antibody-drug conjugate is administered to the individual at a dose of 2.0 mg/kg to 2.5 mg/kg. In some embodiments, the antibody-drug conjugate is administered to the individual at a dose of 1.5 mg/kg. In some embodiments, the antibody-drug conjugate is administered to the individual at a dose of 1.6 mg/kg. In some embodiments, the antibody-drug conjugate is administered to the individual at a dose of 1.7 mg/kg. In some embodiments, the antibody-drug conjugate is administered to the individual at a dose of 1.8 mg/kg. In some embodiments, the antibody-drug conjugate is administered to the individual at a dose of 1.9 mg/kg. In some embodiments, the antibody-drug conjugate is administered to the individual at a dose of 2.0 mg/kg. In some embodiments, the antibody-drug conjugate is administered to the individual at a dose of 2.1 mg/kg. In some embodiments, the antibody-drug conjugate is administered to the individual at a dose of 2.2 mg/kg. In some embodiments, the antibody-drug conjugate is administered to the individual at a dose of 2.3 mg/kg. In some embodiments, the antibody-drug conjugate is administered to the individual at a dose of 2.4 mg/kg. In some embodiments, the antibody-drug conjugate is administered to the individual at a dose of 2.5 mg/kg.

The concentration of a protein-based drug substance can be estimated using various methods that typically reference a comparative standard. Methods and reference standards evolve over time to be more precise and accurate in determining protein concentrations. In some embodiments, the dose is measured using the BSA-based Extinction Coefficient (EC) method. The BSA-based Extinction Coefficient (EC) method is a UV spectrophotometric method for determining the protein concentration using the sample's absorbance and an extinction coefficient (EC). Exemplary protocols are provided in Examples 3 and 4. Briefly, the absorbance of the sample at 280 nm with scatter-correction at 320 nm is read over multiple pathlengths, producing a slope (Abs/mm) which is then used to calculate protein concentration using Beer-Lambert's law and a product-specific extinction coefficient of 1.07 (mg/ml)⁻¹·cm⁻¹.

In some embodiments, the antibody-drug conjugate is administered to the individual at a dose of 1.5 mg/kg, measured using the BSA-based EC method. In other embodiments, the dose contains the same mole-quantity of the drug as 1.5 mg/kg measured using the BSA-based EC method, but is measured using another method and therefore expressed differently, for example, as 1.1 mg/kg dose as measured using a disitamab vedotin-based EC method. In some embodiments, the antibody-drug conjugate is administered to the individual at a dose of 2.0 mg/kg, measured using the BSA-based EC method. In other embodiments, the dose contains the same mole-quantity of the drug as 2.0 mg/kg measured using the BSA-based EC method, but is measured using another method and therefore expressed differently, for example, as 1.5 mg/kg dose as measured using a disitamab vedotin-based EC method. In some embodiments, the antibody-drug conjugate is administered to the individual at a dose of 2.5 mg/kg, measured using the BSA-based EC method. In other embodiments, the dose contains the same mole-quantity of the drug as 2.5 mg/kg measured using the BSA-based EC method, but is measured using another method and therefore expressed differently, for example, as 1.9 mg/kg dose as measured using a disitamab vedotin-based EC method. Briefly, in the disitamab vedotin-based EC method, the absorbance of the sample at 280 nm with scatter-correction at 320 nm is read over multiple pathlengths, producing a slope (Abs/mm) which is then used to calculate protein concentration using Beer-Lambert's law and a different extinction coefficient.

In some embodiments, the antibody-drug conjugate is administered to the individual every 2 weeks or every 14 days (Q2W). In some embodiments, the antibody-drug conjugate is disitamab vedotin, and DV is administered to the individual intravenously (IV) at a dose of 2.0 mg/kg or 2.5 mg/kg, every 2 weeks or every 14 days (Q2W). In some embodiments, the antibody-drug conjugate is disitamab vedotin, and DV is administered to the individual intravenously (IV) at a dose of 2.0 mg/kg or 2.5 mg/kg, every 2 weeks or every 14 days (Q2W) on the same day of each cycle.

In some embodiments, the anti-PD-1 antibody is administered to the individual at a dose of 3.0 mg/kg. In some embodiments, the anti-PD-1 antibody is administered intravenously to the individual. In some embodiments, the anti-PD-1 antibody is administered to the individual every 2 weeks or every 14 days (Q2W). In some embodiments, 3.0 mg/kg of the anti-PD-1 antibody is intravenously administered to the individual every 2 weeks or every 14 days (Q2W). In some embodiments, 3.0 mg/kg of the anti-PD-1 antibody is intravenously administered to the individual every 2 weeks or every 14 days (Q2W) on the same day of each cycle. In some embodiments, the anti-PD-1 antibody is toripalimab. In some embodiments, the anti-PD-1 antibody is toripalimab, and toripalimab is administered to the individual at a dose of 3.0 mg/kg. In some embodiments, the anti-PD-1 antibody is toripalimab, and toripalimab is administered intravenously to the individual at a dose of 3.0 mg/kg, once every 2 weeks, for about 60 minutes.

In some embodiments, the antibody-drug conjugate is disitamab vedotin, and DV is administered to the individual intravenously (IV) at a dose of 2.0 mg/kg or 2.5 mg/kg, every 2 weeks or every 14 days (Q2W), and 3.0 mg/kg of the anti-PD-1 antibody is intravenously administered to the individual every 2 weeks or every 14 days (Q2W). In some embodiments, the antibody-drug conjugate is disitamab vedotin, and DV is administered to the individual intravenously (IV) at a dose of 2.0 mg/kg or 2.5 mg/kg, every 2 weeks or every 14 days (Q2W); the anti-PD-1 antibody is toripalimab, and 3.0 mg/kg of toripalimab is administered intravenously to the individual every 2 weeks or every 14 days (Q2W). In some embodiments, the antibody-drug conjugate is disitamab vedotin, and DV is administered to the individual intravenously (IV) at a dose of 2.0 mg/kg, every 2 weeks or every 14 days (Q2W); the anti-PD-1 antibody is toripalimab, and 3.0 mg/kg of toripalimab is administered intravenously to the individual every 2 weeks or every 14 days (Q2W) on the same day of each cycle. In some embodiments, the antibody-drug conjugate is disitamab vedotin, and DV is administered to the individual intravenously (IV) at a dose of 2.5 mg/kg, every 2 weeks or every 14 days (Q2W); the anti-PD-1 antibody is toripalimab, and 3.0 mg/kg of toripalimab is administered intravenously to the individual every 2 weeks or every 14 days (Q2W) on the same day of each cycle.

In some embodiments, the antibody-drug conjugate is administered sequentially with the anti-PD-1 antibody. In some embodiments, the antibody-drug conjugate is administered prior to the anti-PD-1 antibody. In some embodiments, the antibody-drug conjugate is administered prior to the anti-PD-1 antibody, and wherein the interval between administration of the antibody-drug conjugate and the anti-PD-1 antibody is 30 to 60 minutes.

In some embodiments, the method comprises intravenously administering disitamab vedotin to the individual at 2.5 mg/kg and intravenously administering toripalimab to the individual at 3.0 mg/kg, every 2 weeks or every 14 days, on the same day of each cycle, and the dose of disitamab vedotin is measured using the bovine serum albumin (BSA)-based Extinction Coefficient (EC) method. In some embodiments, the method comprises intravenously administering disitamab vedotin to the individual at 2.5 mg/kg and intravenously administering toripalimab to the individual at 3.0 mg/kg, every 2 weeks or every 14 days, on the same day of each cycle, wherein the antibody-drug conjugate is administered prior to the anti-PD-1 antibody, and wherein the interval between administration of the antibody-drug conjugate and the anti-PD-1 antibody is 30 to 60 minutes. In some embodiments, the method comprises intravenously administering disitamab vedotin to the individual at 2.5 mg/kg and intravenously administering toripalimab to the individual at 3.0 mg/kg, every 2 weeks or every 14 days, on the same day of each cycle, wherein the disitamab vedotin is administered prior to toripalimab, the interval between administration is 30 to 60 minutes, and the dose of disitamab vedotin is measured using the bovine serum albumin (BSA)-based Extinction Coefficient (EC) method.

In some embodiments, the administration of the anti-PD-1 antibody and the antibody-drug conjugate according to the methods of the present disclosure is a second-line (2L) treatment. In some embodiments, the administration of the anti-PD-1 antibody and the antibody-drug conjugate according to the methods of the present disclosure is a third-line or higher (3L+) treatment.

In some embodiments, the method further comprises administration of one or more tertiary agents. In some embodiments, the method comprises administering to the individual an effective amount of an anti-PD-1 antibody, an anti-human epidermal growth factor receptor 2 (HER2) antibody-drug conjugate, and a chemotherapy. In some embodiments, the chemotherapy comprises oxaliplatin and capecitabine (CAPOX). In some embodiments, the anti-PD-1 antibody is toripalimab. In some embodiments, the HER2 antibody-drug conjugate is disitamab vedotin.

In some embodiments, the method comprises administering disitamab vedotin, toripalimab, oxaliplatin, and capecitabine in a 6-week or 42-day cycle. In some embodiments, the method comprises intravenously administering oxaliplatin to the individual at 100 mg/m².

In some embodiments, the method comprises intravenously administering oxaliplatin to the individual Q3W. In some embodiments, the method comprises intravenously administering oxaliplatin to the individual 100 mg/m² Q3W. In some embodiments, the method comprises intravenously administering oxaliplatin to the individual 100 mg/m² Q3W on day 1 of the dosing cycle. In some embodiments, the method comprises orally administering capecitabine to the individual at 750 mg/m². In some embodiments, the method comprises orally administering capecitabine to the individual Q3W. In some embodiments, the method comprises orally administering capecitabine to the individual 750 mg/m² Q3W. In some embodiments, the method comprises orally administering capecitabine to the individual on days 1 to 14 of a dosing cycle 750 mg/m² Q3W. In some embodiments, the capecitabine is administered to the individual once on day 1 of a dosing cycle. In some embodiments, the capecitabine is administered to the individual twice on days 2-14 of a dosing cycle. In some embodiments, the method comprises intravenously administering the anti-PD-1 antibody to the individual at 3 mg/kg Q2W. In some embodiments, the method comprises intravenously administering the HER2 antibody-drug conjugate to the individual at 2.5 mg/kg Q2W. In some embodiments, the method comprises intravenously administering the HER2 antibody-drug conjugate to the individual at 2.0 mg/kg Q2W. In some embodiments, one dosing cycle is 6 weeks or 42 days. In some embodiments, disitamab vedotin, toripalimab, oxaliplatin, and capecitabine are administered in a 6-week or 42-day cycle; wherein disitamab vedotin, toripalimab, oxaliplatin, and capecitabine are administered on day 1 of a 6-week or 42-day cycle; wherein disitamab vedotin is administered at a dose of 2.5 mg/kg intravenously once every 2 weeks (Q2W); wherein toripalimab is intravenously administered at a dose of 3 mg/kg once every 2 weeks (Q2W); wherein oxaliplatin is administered at a dose of 100 mg/m² intravenously once every 3 weeks or every 21 days (Q3W); wherein the capecitabine is administered at a dose of 750 mg/m² orally once on days 1, 15, 22, and 36 of the 6-week or 42-day cycle; wherein the capecitabine is administered at a dose of 750 mg/m² orally twice daily on days 2-14 and 23-35 of the 6-week or 42-day cycle; and wherein the dose of disitamab vedotin is measured using the bovine serum albumin (BSA)-based Extinction Coefficient (EC) method. In some embodiments, disitamab vedotin, toripalimab, oxaliplatin, and capecitabine are administered in a 6-week or 42-day cycle; wherein disitamab vedotin, toripalimab, oxaliplatin, and capecitabine are administered on day 1 of a 6-week or 42-day cycle; wherein disitamab vedotin is administered at a dose of 2.0 mg/kg intravenously once every 2 weeks (Q2W); wherein toripalimab is intravenously administered at a dose of 3 mg/kg once every 2 weeks (Q2W); wherein oxaliplatin is administered at a dose of 100 mg/m² intravenously once every 3 weeks or every 21 days (Q3W); wherein the capecitabine is administered at a dose of 750 mg/m² orally once on days 1, 15, 22, and 36 of the 6-week or 42-day cycle; wherein the capecitabine is administered at a dose of 750 mg/m² orally twice daily on days 2-14 and 23-35 of the 6-week or 42-day cycle; and wherein the dose of disitamab vedotin is measured using the bovine serum albumin (BSA)-based Extinction Coefficient (EC) method. In some embodiments, the individual is HER2-low. In some embodiments, the individual is HER2-positive. In some embodiments, the individual is HER2-0. In some embodiments, the cancer is a HER2-positive, HER2-low, or HER2-zero locally advanced or metastatic gastric cancer. In some embodiments, the cancer is a HER2-positive, HER2-low, or HER2-zero locally advanced or metastatic gastroesophageal junction adenocarcinoma.

In some embodiments, the method comprises administering disitamab vedotin, toripalimab, oxaliplatin, and capecitabine in a 6-week or 42-day cycle. In some embodiments, the method comprises intravenously administering oxaliplatin to the individual at 100 mg/m². In some embodiments, the method comprises intravenously administering oxaliplatin to the individual Q3W. In some embodiments, the method comprises intravenously administering oxaliplatin to the individual 100 mg/m² Q3W. In some embodiments, the method comprises intravenously administering oxaliplatin to the individual 100 mg/m² Q3W on day 1 of the dosing cycle. In some embodiments, the method comprises orally administering capecitabine to the individual at 1000 mg/m². In some embodiments, the method comprises orally administering capecitabine to the individual Q3W. In some embodiments, the method comprises orally administering capecitabine to the individual 1000 mg/m² Q3W. In some embodiments, the method comprises orally administering capecitabine to the individual on days 1 to 14 of a dosing cycle 1000 mg/m² Q3W. In some embodiments, the capecitabine is administered to the individual once on day 1 of a dosing cycle. In some embodiments, the capecitabine is administered to the individual twice on days 2-14 of a dosing cycle. In some embodiments, the method comprises intravenously administering the anti-PD-1 antibody to the individual at 3 mg/kg Q2W. In some embodiments, the method comprises intravenously administering the HER2 antibody-drug conjugate to the individual at 2.5 mg/kg Q2W. In some embodiments, the method comprises intravenously administering the HER2 antibody-drug conjugate to the individual at 2.5 mg/kg Q2W, and the individual is HER2-low. In some embodiments, the method comprises intravenously administering the HER2 antibody-drug conjugate to the individual at 2.0 mg/kg Q2W. In some embodiments, disitamab vedotin, toripalimab, oxaliplatin, and capecitabine are administered in a 6-week or 42-day cycle; wherein disitamab vedotin, toripalimab, oxaliplatin, and capecitabine are administered on day 1 of a 6-week or 42-day cycle; wherein disitamab vedotin is administered at a dose of 2.5 mg/kg intravenously once every 2 weeks (Q2W); wherein toripalimab is intravenously administered at a dose of 3 mg/kg once every 2 weeks (Q2W); wherein oxaliplatin is administered at a dose of 100 mg/m² intravenously once every 3 weeks or every 21 days (Q3W); wherein the capecitabine is administered at a dose of 1000 mg/m² orally once on days 1, 15, 22, and 36 of the 6-week or 42-day cycle; wherein the capecitabine is administered at a dose of 1000 mg/m² orally twice daily on days 2-14 and 23-35 of the 6-week or 42-day cycle; and wherein the dose of disitamab vedotin is measured using the bovine serum albumin (BSA)-based Extinction Coefficient (EC) method. In some embodiments, disitamab vedotin, toripalimab, oxaliplatin, and capecitabine are administered in a 6-week or 42-day cycle; wherein disitamab vedotin, toripalimab, oxaliplatin, and capecitabine are administered on day 1 of a 6-week or 42-day cycle; wherein disitamab vedotin is administered at a dose of 2.0 mg/kg intravenously once every 2 weeks (Q2W); wherein toripalimab is intravenously administered at a dose of 3 mg/kg once every 2 weeks (Q2W); wherein oxaliplatin is administered at a dose of 100 mg/m² intravenously once every 3 weeks or every 21 days (Q3W); wherein the capecitabine is administered at a dose of 1000 mg/m² orally once on days 1, 15, 22, and 36 of the 6-week or 42-day cycle; wherein the capecitabine is administered at a dose of 1000 mg/m² orally twice daily on days 2-14 and 23-35 of the 6-week or 42-day cycle; and wherein the dose of disitamab vedotin is measured using the bovine serum albumin (BSA)-based Extinction Coefficient (EC) method. In some embodiments, one dosing cycle is 6 weeks or 42 days. In some embodiments, the individual is HER2-low. In some embodiments, the individual is HER2-positive. In some embodiments, the individual is HER2-0. In some embodiments, the cancer is a HER2-positive, HER2-low, or HER2-zero locally advanced or metastatic gastric cancer. In some embodiments, the cancer is a HER2-positive, HER2-low, or HER2-zero locally advanced or metastatic gastroesophageal junction adenocarcinoma.

In some embodiments, the anti-PD-1 antibody, HER2 antibody-drug conjugate and chemotherapy are administered sequentially. In some embodiments, the HER2 antibody-drug conjugate is administered first, followed by anti-PD-1 antibody, followed by the chemotherapy. In some embodiments, the HER2 antibody-drug conjugate is infused over between about 30 minutes to about 90 minutes, preferably 60 minutes. In some embodiments, the anti-PD-1 antibody is infused over about 60 minutes or 30 minutes. In some embodiments, the interval between the HER2 antibody-drug conjugate is between about 30 minutes to about 60 minutes. In some embodiments, the chemotherapy is oxaliplatin and capecitabine (CAPOX). In some embodiments, the start of oxaliplatin infusion is about 60 minutes after the end of the anti-PD-1 antibody infusion. In some embodiments, the oxaliplatin is infused over between about 3 hours to about 4 hours. In some embodiments, the capecitabine is administered orally. In some embodiments, the capecitabine is taken orally on day 1 of the dosing cycle. In some embodiments, the capecitabine is taken once orally on day 1 of the dosing cycle. In some embodiments, the capecitabine is taken twice orally from day 2 to day 14 of the dosing cycle. In some embodiments, the interval between two oral administrations of capecitabine is between about 10 hours to about 12 hours. In some embodiments, the capecitabine is administered once in the evening and once in the morning of the same day.

In some embodiments, the administration of the anti-PD-1 antibody, the chemotherapy and the antibody-drug conjugate according to the methods of the present disclosure is a first-line (1L) treatment. In some embodiments, the individual has locally advanced or metastatic gastric cancer; or a locally advanced or metastatic gastroesophageal junction cancer, e.g., gastroesophageal junction adenocarcinoma.

In some embodiments, the method further comprises administration of one or more secondary agents. In some embodiments, the method comprises administering to the individual an effective amount of an anti-PD-1 antibody, an anti-human epidermal growth factor receptor 2 (HER2) antibody-drug conjugate, and an anti-HER2 antibody. In some embodiments, the anti-HER2 antibody is trastuzumab.

In some embodiments, the method comprises administering disitamab vedotin, toripalimab, and trastuzumab in a 6-week or 42-day cycle. In some embodiments, the method comprises intravenously administering the anti-HER2 antibody to the individual at an initial loading dose of 8 mg/kg. In some embodiments, the method comprises intravenously administering the anti-HER2 antibody to the individual at a dose of 6 mg/kg. In some embodiments, the method comprises intravenously administering anti-HER2 antibody to the individual Q3W. In some embodiments, the method comprises intravenously administering oxaliplatin to the individual 6 mg/kg Q3W. In some embodiments, the method comprises intravenously administering the anti-PD-1 antibody to the individual at 3 mg/kg Q2W. In some embodiments, the method comprises intravenously administering the HER2 antibody-drug conjugate to the individual at 2.5 mg/kg Q2W. In some embodiments, one dosing cycle is 6 weeks or 42 days. In some embodiments, the individual is HER2-positive. In some embodiments, the anti-HER2 antibody is trastuzumab. In some embodiments, disitamab vedotin, toripalimab, and trastuzumab are administered in a 6-week or 42-day cycle; wherein disitamab vedotin, toripalimab, and trastuzumab are administered on day 1 of a 6-week or 42-day cycle; wherein disitamab vedotin is administered at a dose of 2.5 mg/kg intravenously once every 2 weeks (Q2W); wherein toripalimab is administered at a dose of 3 mg/kg intravenously once every 2 weeks (Q2W); wherein trastuzumab is administered at an initial dose of 8 mg/kg on day 1 of the 6-week or 42-day cycle; wherein trastuzumab is administered at a dose of about 6 mg/kg every 3 weeks (Q3W) starting about week 3 of the 6-week or 42-day cycle; and wherein the dose of disitamab vedotin is measured using the bovine serum albumin (BSA)-based Extinction Coefficient (EC) method. In some embodiments, the individual is HER2-positive. In some embodiments, the cancer is a HER2-positive, locally advanced or metastatic gastric cancer. In some embodiments, the cancer is a HER2-positive locally advanced or metastatic gastroesophageal junction adenocarcinoma.

In some embodiments, the anti-PD-1 antibody, the HER2 antibody-drug conjugate and the anti-HER2 antibody are administered sequentially. In some embodiments, the HER2 antibody-drug conjugate is administered first, followed by anti-PD-1 antibody, followed by the anti-HER2 antibody. In some embodiments, the HER2 antibody-drug conjugate is infused over between about 30 minutes to about 90 minutes, preferably 60 minutes. In some embodiments, the anti-PD-1 antibody is infused over about 60 minutes or 30 minutes. In some embodiments, the interval between the HER2 antibody-drug conjugate is between about 30 minutes to about 60 minutes. In some embodiments, the anti-HER2 antibody is trastuzumab. In some embodiments, the start of anti-HER2 antibody infusion is about 60 minutes after the end of the anti-PD-1 antibody infusion. In some embodiments, the anti-HER2 antibody is infused over between about 90 minutes or 30 minutes. In some embodiments, the anti-HER2 antibody is trastuzumab.

In some embodiments, the administration of the anti-PD-1 antibody, the anti-HER2 antibody and the antibody-drug conjugate according to the methods of the present disclosure is a first-line (1L) treatment. In some embodiments, the individual has locally advanced or metastatic gastric cancer; or a locally advanced or metastatic gastroesophageal junction cancer, e.g., gastroesophageal junction adenocarcinoma.

In some embodiments, administration of the anti-PD-1 antibody and the antibody-drug conjugate to an individual with gastric cancer or GEJC results in an overall survival (OS) of 14 months or longer.

In some embodiments, administration of the anti-PD-1 antibody and the antibody-drug conjugate to an individual with gastric cancer or GEJC results in progression-free survival (PFS) of 5 months or longer.

In some embodiments, the administration of the anti-PD-1 antibody and the antibody-drug conjugate to an individual with breast cancer results in progression-free survival (PFS) of 9 months or longer. In some embodiments, the breast cancer is hormone receptor (HR)+HER2-low expressing breast cancer, and the method comprises intravenously administering disitamab vedotin to the individual at 2.5 mg/kg and intravenously administering toripalimab to the individual at 3.0 mg/kg, every 2 weeks or every 14 days, on the same day of each cycle.

In some embodiments, the administration of the anti-PD-1 antibody and the antibody-drug conjugate to an individual with breast cancer results in progression-free survival (PFS) of 4 months or longer. In some embodiments, the breast cancer is hormone receptor (HR)+HER2-low expressing breast cancer, and the method comprises intravenously administering disitamab vedotin to the individual at 2.0 mg/kg and intravenously administering toripalimab to the individual at 3.0 mg/kg, every 2 weeks or every 14 days, on the same day of each cycle.

In some embodiments, administration of the anti-PD-1 antibody, the antibody-drug conjugate, and the chemotherapy to a population of individuals with HER2-positive gastric cancer or GEJC results in an overall response rate of 73% or greater.

In some embodiments, administration of the anti-PD-1 antibody, the antibody-drug conjugate, and the chemotherapy to a population of individuals with HER2-low gastric cancer or GEJC results in an overall response rate of 67% or greater.

In some embodiments, administration of the anti-PD-1 antibody, the antibody-drug conjugate, and the anti-HER2 antibody to a population of individuals with HER2-positive gastric cancer or GEJC results in an overall response rate of 94% or greater.

In some embodiments, administration of the anti-PD-1 antibody and the antibody-drug conjugate results in a complete response (CR) or partial response (PR) in the individual. In some embodiments, administration of the anti-PD-1 antibody, the HER2 antibody, and the antibody-drug conjugate results in a complete response (CR) or partial response (PR) in the individual. In some embodiments, administration of the anti-PD-1 antibody, a chemotherapy, and the antibody-drug conjugate results in a complete response (CR) or partial response (PR) in the individual. In some embodiments, response to treatment according to the methods disclosed herein is evaluated using RECIST v1.1 criteria. Exemplary criteria are provided below.

Response Evaluation Criteria for Solid Tumors (RECIST) Version 1.1
(Eisenhauer, E. A. et al. (2009) Eur J Cancer 45(2): 228-247)

| Term | Definition |
|---|---|
| Complete Response (CR) | Disappearance of all target lesions. Any pathological lymph nodes (whether |
| | target or non-target) must have reduction in short axis to <10 mm |
| | Cannot have previously met criteria for PD |
| | Confirmation required with repeat scans at least 4 weeks after first |
| | documentation of response |
| Partial Response (PR) | A ≥30% decrease in the sum of diameters of target lesions (longest for |
| | non-nodal target lesions and the short axes for nodal target lesions), taking as |
| | reference the baseline sum diameters |
| | Cannot have previously met criteria for PD |
| | Confirmation required with repeat scans at least 4 weeks after first |
| | documentation of response |
| Stable Disease (SD) | Neither sufficient shrinkage to qualify for PR nor sufficient increase to qualify |
| | for PD, taking as reference the smallest sum diameters while on study |
| | Cannot have previously met criteria for PD |
| | Confirmation is not required |
| Progressive Disease (PD) | A ≥20% relative increase in the sum of diameters of target lesions, taking as |
| | reference the smallest sum on study (including baseline sum if that is the |
| | smallest on study). In addition, the sum must also demonstrate an absolute |
| | increase ≥5 mm |
| | The appearance of 1 or more new lesions is also considered progression |
| | Confirmation is not required unless equivocal |

In some embodiments, the individual is a human.

### III. Kits and Articles of Manufacture

In another aspect, an article of manufacture or kit is provided which comprises an anti-HER2 antibody-drug conjugate as described herein. The article of manufacture or kit may further comprise instructions for use of the antibody-drug conjugate in the methods of the present disclosure, e.g., for administering an effective amount of an anti-PD-1 antibody and the antibody-drug conjugate to an individual in need thereof according to any one of the methods disclosed herein. In some embodiments, the article of manufacture or kit further comprises toripalimab. In some embodiments, the article of manufacture or kit further comprises toripalimab and oxaliplatin and capecitabine (CAPOX). In some embodiments, the article of manufacture or kit further comprises toripalimab and trastuzumab. Thus, in certain embodiments, the article of manufacture or kit comprises instructions for the use of treating or preventing progression of cancer in an individual, e.g., according to any one of the methods disclosed herein.

The article of manufacture or kit may further comprise a container. Suitable containers include, for example, bottles, vials (e.g., dual chamber vials), syringes (such as single or dual chamber syringes) and test tubes. The container may be formed from a variety of materials such as glass or plastic. The container holds the formulation.

The article of manufacture or kit may further comprise a label or a package insert, which is on or associated with the container, may indicate directions for reconstitution and/or use of the formulation. The label or package insert may further indicate that the formulation is useful or intended for intravenous or other modes of administration. The container holding the formulation may be a single-use vial or a multi-use vial, which allows for repeat administrations of the reconstituted formulation. The article of manufacture or kit may further comprise a second container comprising a suitable diluent. The article of manufacture or kit may further include other materials desirable from a commercial, therapeutic, and user standpoint, including other buffers, diluents, filters, needles, syringes, and package inserts with instructions for use.

In a specific embodiment, the present invention provides kits for a single dose-administration unit. Such kits comprise a container of an aqueous formulation of therapeutic antibody, including both single or multi-chambered pre-filled syringes. Exemplary pre-filled syringes are available from Vetter GmbH, Ravensburg, Germany.

The article of manufacture or kit herein optionally further comprises a container comprising a second medicament, e.g., toripalimab. The article of manufacture or kit herein optionally further comprises a container comprising a third medicament. In some embodiments, the third medicament is an anti-HER2 antibody, e.g., trastuzumab. In some embodiments, the third medicament is a chemotherapy, e.g., oxaliplatin and capecitabine (CAPOX).

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Phase I Clinical Study Evaluating the Safety, Tolerability, Pharmacokinetics and Initial Efficacy of Disitamab Vedotin in Combination with Toripalimab in Treatment of Patients with HER2-Expressing Locally Advanced or Metastatic Solid Tumors

This example describes a phase I clinical study evaluating the safety, tolerability, pharmacokinetics and initial efficacy of disitamab vedotin in combination with toripalimab in treatment of patients with HER2-expressing locally advanced or metastatic solid tumors.

An estimated 90-192 participants are to be enrolled. Interim analysis of study results from 56 subject is described in Example 2.

### Design

This example describes an open-label, phase I clinical study to evaluate the efficacy and safety of disitamab vedotin in combination with toripalimab in subjects with HER2-expressing solid tumors.

The study includes a dose escalation phase and a dose extension phase. The preset disitamab vedotin dose groups in the dose escalation phase are 2.0 mg/kg Q2W and 2.5 mg/kg Q2W; the preset toripalimab dose group in the dose escalation phase is 3 mg/kg Q2W. The dose escalation uses a 3+3 design.

At first, three qualified subjects will be enrolled. If dose limiting toxicity (DLT) during the DLT evaluation period is not observed in the three subjects in the 2.0 mg/Kg dose group, they can be escalated to the next dose group. If ⅓ of the subjects (1 case) in the 2.0 mg/kg dose group experience DLT, 3 additional subjects need to be added for further observation. If no DLT occurs in the three additional subjects, they can continue to escalate to the next dose group. If one or more of the three additional subjects experience DLT, the dose escalation will be stopped and the study will be withdrawn to the previous cohort. (If the current group is 2.0 mg/kg, it is reduced to 1.5 mg/kg). If the dose is escalated to the 2.5 mg/kg cohort and the subjects don't meet the DLT standard within 28 days of the first dose, the dose escalation will not be continued to observe DLT and MTD.

At the end of the dose escalation phase, the investigator will determine the recommended Phase II dose (RP2D) for each cohort based on the available safety, tolerability, PK and efficacy, and apply RP2D to enter the dose extension phase.

Her2-expressing solid tumors will be included in the dose escalation phase. The HER2 expression is defined as follows (conforming to one of the following):

$$HER 2 IHC 3 + , 2 + , 1 + ;$$

HER2 gene amplification (ISH+: HER2/CEP17 Rate >2), If the subject is breast cancer, refer to the criteria of breast cancer gene amplification.

The dose extension phase will include 6 cohorts of solid tumors, and the HER2 expression criteria are consistent with the dose escalation phase. Cohort 1 and Cohort 6 are shown below:
Cohort 1: First-line (i.e., line one therapy) treatment progresses for HER2-expressing gastric/gastroesophageal junction cancer (GC/GEJ);
Cohort 2: HR-negative, HER2-low expressing, unresectable locally advanced or metastatic breast cancer
Cohort 3: Standard therapy progressed HR-positive, HER2-low expressing, unresectable locally advanced or metastatic breast cancer
Cohort 6: Standard treatment progresses in other solid tumors, such as endometrial cancer, with HER2 expression.

Subjects who have previously received HER2-targeted therapy such as trastuzumab in the dose escalation or extension phase should provide tumor tissue samples collected after the failure of HER2-targeted therapy to the research center for the purpose of determining HER2 expression status. All subjects are required to submit slides during the screening period for the HER2 status review by the research center laboratory (if the previous HER2 test is for the research center laboratory and the investigator is approved, the HER2 status review is not required).

DLT definition: Within 28 days after the first dose, the investigator determines that the following toxicity may or must be related to disitamab vedotin and/or toripalimab occurred in the subject:
Grade 4 neutropenia lasts for more than 48 hours; . . . .
Febrile Neutropenia (neutrophil absolute value<1000/mm3 accompanied by fever, body temperature greater than 38.37 or sustained fever, body temperature continuously higher than 38° C. for more than 1 hour);
Grade 3 neutropenia with evidence of infection;
Grade 3 thrombocytopenia with bleeding tendency;
Grade 4 thrombocytopenia;
Non-hematological toxicity of grade 3 or above after supportive treatment, except for nausea, vomiting, and alopecia;
Neurotoxicity ≥Grade 2, persisting without remission until next dose;
Cardiotoxicity grade ≥2;
An increase in alanine aminotransferase (ALT) or aspartate aminotransferase (AST) of grade 3 or above, or an increase in ALT/AST of grade 2 or above accompanied by an increase in bilirubin of grade 2 or above, except for the following:
   a) Transient is not defined as DLT according to the judgment of the researcher (such as the duration of less than 7 days);
   b) Subjects with the baseline elevation of ALT or AST at Grade 2 and the elevation below 10 times the upper limit of normal range (ULN) after treatment are not defined as DLT.

If subjects experience DLT during the observation period of DLT, the investigator can determine whether to accept subsequent study drug treatment, reduce or discontinue dose according to the dose adjustment principles specified in the protocol (dose adjustment is not allowed for toripalimab).

All subjects will receive treatment with disitamab vedotin in combination with toripalimab until progressive disease, death, intolerable toxicity, or other termination criteria specified in the protocol are met, whichever occurs first. But the maximum duration of treatment with toripalimab is 24 months.

During the study, the researchers will use RECIST 1.1 for tumor evaluation, tumor imaging evaluation will be conducted every 6 weeks (±7 days) starting from the first dose, and every 12 weeks (±7 days) after 48 weeks until progressive disease, death, intolerable toxicity, or other termination criteria specified in the protocol are met, whichever occurs first.

After the first judgment of progressive disease based on the RECIST 1.1 standard by researchers, if the subject is clinically stable and does not experience intolerable toxicity during treatment, the subject is allowed to continue receiving toripalimab treatment until the progressive disease is confirmed. Clinical stability is defined as: stable ECOG score, no unacceptable toxicity associated with toripalimab treatment, no rapid progressive disease requiring other salvage treatments, and no emergency requiring emergency medical intervention due to progressive disease (such as central nervous system metastasis, tumor compression of the airway leading to dyspnea or spinal cord compression, etc.).

Collecting PK and ADA blood samples to evaluate pharmacokinetic and immunogenicity during the study: Cohorts 1 and 6 adopt a sparse blood sampling design, wherein the peak and valley concentrations of disitamab vedotin and MMAE will be collected. PK and ADA samples will be collected from at least 10 subjects in each cohort.

The experimental drugs include disitamab vedotin and toripalimab. disitamab vedotin is to be administered at a dose of 2.0 mg/kg or 2.5 mg/kg or 1.5 mg/kg by intravenous drip, once every two weeks. Toripalimab is to be administered at a dose of 3.0 mg/kg by intravenous drip, once every 2 weeks, for approximately 60 minutes.

During the study, on the day of treatment with the combination of disitamab vedotin and toripalimab, disitamab vedotin should be administered by infusion followed by intravenous drip of toripalimab at intervals of 30 to 60 minutes.

### Objectives and Outcomes

In this study, the primary objective is to evaluate the safety of disitamab vedotin combined with toripalimab in subjects with HER2-expressing locally advanced or metastatic solid tumors. The primary outcomes include:
Dose escalation phase: dose-limiting toxicity, DLT;
Dose extension phase: The objective response rate (ORR) and duration of response (DOR) determined by researchers based on the RECIST 1.1 standard.

The secondary objective is to evaluate the efficacy of disitamab vedotin combined with toripalimab in subjects with HER2-expressing locally advanced or metastatic solid tumors. The secondary outcomes include:
The 6-month and 12-month progression free survival rate (PFSR) and clinical benefit rate (CBR; defined as CR, PR, or SD>24 weeks) determined by researchers based on the RECIST 1.1 standard;
Overall survival rate (OS rate) at 6 and 12 months;
The frequency and severity of adverse events (AE) (NCICTCAE5.0); Changes in vital signs, physical examination, electrocardiogram, safety laboratory indicators;
Correlation between biomarkers (HER2IHC, HER2 amplification, HER2 mutation. (limited to non-small cell lung cancer subjects), etc.) and clinical efficacy parameters (ORR, CBR, PFSR, etc.);
PK parameters of disitamab vedotin and toripalimab;
Frequency and titer of anti-disitamab vedotin antibody (ADA); Frequency of neutralizing anti-disitamab vedotin antibody (NADA) in ADA-positive subjects; and.
Frequency and titer of anti-toripalimab antibody (ADA); Frequency of neutralizing anti-toripalimab antibody (NADA) in ADA-positive subjects.

### Inclusion/Exclusion Criteria

Subjects are eligible to be included in the study only if all the following criteria are met:
The subjects can understand the informed consent form, voluntarily participate and sign the informed consent form;
The subjects are ≥18 years old on the date of signing the informed consent form, regardless of gender;
Diagnosed histologically or cytologically with metastatic or locally advanced unresectable HER2-expressing solid tumors;
Patients who have failed first-line (e.g., line one therapy) or standard treatment (progressive disease or intolerable toxic side effects after treatment) or have no effective treatment methods;
At least 1 measurable lesion at baseline according to RECIST 1.1 criteria. If the subject has only one measurable lesion at baseline, the lesion area must have not received radiation therapy in the past, or there is evidence of significant progression after radiation therapy (limited to dose extension phase);
Having an ECOG performance status score of 0 or 1;
LVEF≥50%, measured by Echocardiogram (ECHO), only measured by cardiac Multigated Radionuclide Angiography (MUGA) when there is no ECHO. Baseline and follow-up measurements remain consistent;
NYHA cardiac function grading<3;
Liver function within 7 days prior to first dose meets the following criteria:
   Total bilirubin≤1.5×ULN (total bilirubin≤3×ULN in subjects with Gilbert's syndrome or liver metastasis);
   Transaminase (ALT/AST)≤2.5×ULN (liver metastasis subjects≤5×ULN);
Renal function within 7 days before first dose: serum muscle ff<1.0×ULN and serum creatinine clearance rate ≥60 mL/min (calculated according to Cockcroft-Gault formula);
Bone marrow function within 7 days prior to first dose meets the following criteria:
   Hemoglobin≥9.0 g/dL;
   Absolute neutrophil count≥1.5×109/L;
   Platelets count≥100×109/L;
   International Normalized ratio (INR)≤1.5 or partially activated prothrombin time (APTT)≤1.5×ULN;
If the subjects have received blood transfusion treatment, the test results after 28 days of transfusion must meet the above criteria;
Expected survival≥3 months;
Female subjects with fertility or male subjects whose partners with fertility agree to use highly effective contraception from 7 days before the first dose until 24 weeks after the last dose. Female subjects with fertility must have a negative serum pregnancy test within 7 days prior to the first dose, and subjects with false positive results who are confirmed not to be pregnant are eligible to participate in the study;
The subjects are capable and willing to comply with the visit, treatment plan, laboratory examination, and other research related procedures stipulated in the research protocol.

Subjects who meet any of the following criteria must be excluded from this study plan:
Subjects with untreated active brain metastasis or meningeal metastasis; If the subject's brain metastasis has been treated and the condition of the metastasis is stable (brain imaging examination at least 4 weeks before the first dose shows that the lesion is stable and there are no new neurological symptoms, or the neurological symptoms have returned to baseline level), and there is no evidence of new or original brain metastasis enlargement, admission is allowed;
Participated in any other interventional clinical trials within 28 days before the first dose;
Received other anti-tumor treatments within 5 half-lives or 2 weeks prior to the first dose (Chemotherapy, targeted therapy, biological immunotherapy, traditional Chinese medicine with clear anti-tumor ingredients in the instructions);
Received major surgery within 28 days prior to the first dose (such as abdominal, thoracic and other major surgery; excluding diagnostic puncture or peripheral vascular pathway replacement);
Received radical radiotherapy within 3 months before the first dose; Allow for palliative radiotherapy 2 weeks before dose, with radiation doses that meet local palliative treatment standards and radiation coverage less than 30% of the bone marrow area;
Previously received immune checkpoint blockers, T cell co-stimulatory drugs or ADC drugs, including but not limited to PD-1, PD-L1, CTLA4, LAG3 and other immune checkpoint blockers, therapeutic vaccines, T-DM1, etc., excluding non-squamous non-small cell lung cancer and biliary tumors previously treated with PD-1/PD-L1 (excluding toripalimab);
Subjects need to receive systemic corticosteroids (>10 mg/day of prednisone, or equivalent of other corticosteroids) or immunosuppressant therapy within 14 days before the first dose; Excluding inhalation or topical use of hormones, or receiving physiological replacement doses of hormone therapy due to adrenal insufficiency;
Received live vaccine (including attenuated live vaccine) within 28 days before the first dose;
Having history of any active autoimmune disease or autoimmune disease, including but not limited to interstitial pneumonia, uveitis, inflammatory bowel disease, hepatitis, pituitary inflammation, vasculitis, systemic lupus erythematosus, etc. (leukorrhea and psoriasis that do not need systematic treatment in recent 2 years, hypothyroidism that can be controlled only by hormone replacement therapy, and type I diabetes patients who only need insulin replacement therapy can be included);
Suffering from uncontrolled comorbidities, including but not limited to the following:
   Active HBV or HCV infection;
   History of HIV infection or AIDS;
   Active Tuberculosis;
   Active infection or unexplained fever (body temperature>38.5° C.) occurring during screening and before first dose;
   Suffering from systemic diseases without stable control, including diabetes, hypertension, pulmonary fibrosis, acute lung disease, interstitial lung disease, liver cirrhosis, angina pectoris, severe arrhythmia, etc.;
Subjects who have a prior history of interstitial pneumonia requiring steroid treatment, or are currently in the stage of interstitial pneumonia, or are suspected of having interstitial pneumonia;
Toxicity associated with previous anti-tumor treatments did not return to ≤WCTCAE1 grade, except for alopecia;
History of allogeneic bone marrow or organ transplantation;
Hypersensitivity or delayed anaphylaxis to certain components or similar drugs of disitamab vedotin and toripalimab injection;
Pregnant and/or lactating women;
Other conditions considered by the investigator that may affect the safety or compliance to the drugs in this study, including but not limited to moderate to excessive pleural/ascites/pericardial effusion, difficult-to-correct pleural/ascites/pericardial effusion, and psychiatric disorders, etc.

### Termination of Treatment

Criteria for completion of study treatment:
Progressive disease (including clinical progression), unless the subject meets the criteria for continued treatment after the progressive disease specified in the protocol (limited to continued use of toripalimab);
Death;
The whole study is over.

Criteria for early termination of study treatment:
The subjects are still unable to tolerate toxicity after dose adjustment according to the protocol;
The subjects or their legal representative requests termination of treatment;
The researchers considered that the study treatment should be terminated from the perspective of the best benefit to the subjects;
Pregnancy of subjects during the study period;
Subjects have poor compliance or have received new anti-tumor treatment during the study, making it difficult to comply with the study protocol;
Subject loss of follow-up;
Early study termination.

### Sample Size Calculation and Statistical Analysis

### Sample Size Calculation

The study is divided into dose escalation phase and dose extension phase. The dose escalation phase uses a 3+3 design, according to the safety evaluation results of previous studies, and it is expected to evaluate two cohorts (dose groups) with a sample size of 6-12 cases. The dose extension phase will explore six cohorts, each of which is expected to require 14-30 subjects (including those in the dose escalation phase). A total of 90-192 subjects are expected to this treatment.

### Statistical Analysis

The results of the study mainly used descriptive statistical methods. The measurement data lists the number of people, mean, standard deviation, median, maximum, and minimum. List the frequency and percentage of count data and level data.

### Safety Analysis

All subjects who have used the study drug at least once will be included in the safety analysis (i.e., the safety set). The safety evaluation includes adverse events, DLT, laboratory tests, vital signs, electrocardiogram, left ventricular ejection fraction, ECOG, and immunogenicity. Adverse events are graded according to NCI CTCAE 5.0. Encode adverse events using the International Medical Terminology Dictionary (MedDRA). Summarize the number and frequency of adverse events based on the classification of human system organs and corresponding preferred terminology. DLT adopts list. Compare changes in laboratory examination results, vital signs, electrocardiogram, and ECOG before and after treatment. Display a list of clinically significant outliers. Descriptive statistics are used for the incidence and titer of anti-drug antibodies (ADA) to toripalimab, and explore the potential correlation between immunogenicity and safety.

### Efficacy Analysis

The efficacy analysis defined the evaluable population based on tumor efficacy as all subjects who had used the study drug and had measurable lesions at baseline. Calculate ORR, DCR and CBR respectively, and calculate the 95% exact confidence interval by Clopper-Pearson. For time to event variables including DOR, PFS and OS, if the data permit, using Kaplan-Meier to estimate the median value and 95% confidence interval, PFS rate and OS rate at 6 months, 12 months and the time point of interest.

### Pharmacokinetic Analysis

Descriptively analyze the blood concentration of each detected component at different time points, and plot average and individual c-t curves and semi-logarithmic c-t curves by groups. Plot box diagram of peak and valley concentrations. Calculate PK parameters by noncompartmental analysis, and descriptively analyze each parameter by groups.

### Immunogenicity Analysis

Analyzing the number and percentage of RC48 and/or toripalimab and neutralizing antibody positive subjects at each time point by group.

### Example 2: Interim Analysis of Phase I Clinical Study Results of RC-48 Combined with Toripalimab in the Treatment of HER2 Expressing Locally Advanced or Metastatic Solid Tumors

Antibody-drug conjugate (ADC) significantly improved objective response rates and survival outcomes in patients with solid tumors such as HER2-positive or low-expressing gastric/gastroesophageal junction (G/GEJ) adenocarcinoma. Preclinical data suggest that RC-48 and PD-1 inhibitors have synergistic antitumor effects. To evaluate the safety and efficacy of RC48 in combination with the PD-1 antibody toripalimab in patients with HER2-expressing advanced gastric cancer and other solid tumors, a multi-center, Phase I clinical trial (NCT04280341) was conducted. The current Example describes the results of the clinical trial, the detailed protocol of which is described in Example 1.

### Methods

A multicenter, Phase I clinical trial (NCT04280341), consisting of two phases of dose escalation and dose expansion. The study population was advanced gastric cancer or other solid tumors with HER2 expression (IHC≥1 or ISH positive) that had failed first-line (i.e., line one therapy) or standard therapy. The dose escalation phase of this study adopted a “3+3” design, and the preset RC48 dose groups were 2.0 mg/kg Q2W and 2.5 mg/kg Q2W. The default dose group for toripalimab is 3 mg/kg Q2W. The primary endpoint was objective response rate (ORR).

### Results

A total of 56 patients entered the study and received at least one combination, including 30 patients with stomach cancer and 26 patients with other solid tumors. Median age 58 years (range: 22-75 years); More than half of the patients (30 cases, 53.6%) received at least second-line (e.g., line two therapy) of treatment; HER2 immunohistochemical tests (IHC) were 3+, 2+ and 1+ in 16 (28.6%), 30 (53.6%) and 10 (17.9%) patients, respectively. CPS≥1 in 12 cases (21.4%), CPS <1 in 16 cases (28.6%). There were no dose-limiting toxic effects in the dose escalation phase. Combined with safety and efficacy, it was decided to use RC48 2.5 mg/kg+toripalimab 3.0 mg/kg Q2W as the recommended dose for phase II. The most common treatment-related adverse events (TRAE) were white blood cell count decreased (60.7%), neutrophil count decreased (58.9%), AST increased (46.4%), and ALT increased (44.6%). Among the grade 3-5 TRAE were neutrophil count decreased (21.4%) and white blood cell count decreased (14.3%). As of Feb. 22, 2023, 52 patients had completed efficacy evaluation. In patients with gastric cancer receiving RC48 2.5 mg/kg+toripalimab 3 mg/kg (n=22), the ORR was 50.0% (95% CI 28.2, 71.8) and the median PFS was 5.13 months (95% CI 1.35, 7.75). The median OS was 14.1 months (95% CI 6.2, NR). Excellent results were also observed in other solid tumors treated with the RC48+toripalimab combination, including breast cancer (ORR=38.5%, 5/13) and endometrial cancer (ORR=100%, 1/1).

Overall, RC48 combined with toripalimab showed good safety and excellent efficacy in the treatment of HER2-expressing advanced gastric cancer and other solid tumors.

### Example 3: Bovine Serum Albumin (BSA)-Based Extinction Coefficient (EC) Method

This example describes a method used to determine the concentration of disitamab vedotin using a bovine serum albumin-based (BSA-based) UV spectrophotometric method that determines the protein concentration using the absorbance of the sample and an extinction coefficient (EC).

The Lowry colorimetric method was previously used to determine disitamab vedotin concentration using BSA as the reference standard. Alternatively, UV spectrophotometric methods were also previously developed to determine the protein concentration using the absorbance of the sample and an extinction coefficient. The BSA-based extinction coefficient method used in the present application uses an EC value that was established mathematically to align the Lowry colorimetric and UV spectrophotometric methods, such that both yield the same estimated measurements of disitamab vedotin protein concentration.

Briefly, the sample is transferred to a vessel, and the absorbance at 280 nm with scatter-correction at 320 nm is read over multiple pathlengths using a SoloVPE system. This produces a slope (Abs/mm) which is then used to calculate protein concentration using Beer's law and a product-specific extinction coefficient.

### Calculations and Data Analysis:

The concentration will be calculated by the SoloVPE software, and results can be included in a report generated by the software. Alternatively, calculate the protein concentration using the following equation:

$$Concentration = \frac{{m}_{280}}{ε} \times \frac{10 mm}{1 cm}$$

$$Where : {m}_{280} = slope at 280 nm ( {mm}^{- 1} )$$

$$ε =$$

Calculate the average concentration from the duplicate or triplicate measurements to three decimal places.
For lyophilized samples: Calculate drug product content for lyophilized product vials using the following equation:

$$Drug product content = Concentration ( mg / mL ) \times Final Reconstitution Volume ( mL / vial )$$

### Example 4: Concentration Calculation of Disitamb Vedotin

### Introduction to Methods

This describes the method for a universal protein concentration assay, wherein the protein has the maximum UV absorption at 280 nm. Measure the absorbance value of the sample at this wavelength, and the absorbance value at 280 nm is proportional to the protein concentration, which is calculated according to the Lambert-Beer law.

### Main Equipment

UV-Vis spectrophotometer PE model: Lambda 365

### Analysis Process

The sample was diluted to 0.5±0.1 mg/ml with ultrapure water (the dilution multiple was determined according to the labeled sample concentration, and the dilution multiple≤10 each time), and the final volume of the sample after dilution ≥1 ml. Two sample solutions were prepared in parallel, labeled A and B respectively, diluted and then measured the absorbance of A and B solutions at 260 nm, 280 nm, 320 nm, 325 nm, 330 nm, 335 nm, 340 nm, 345 nm, 350 nm, and each solution was measured twice in parallel.

### Data Analysis

### Concentration Calculation

The formula for calculating protein concentration of the sample solution is listed below, and the average protein concentration of the two parallel solutions is used as the final value.

$$protein concentration ( mg / ml ) = \frac{{A}_{280}}{1.07} \times sample dilution multiple$$

$$Where : 1.07 = the extinction coefficient value ( {( mg / ml )}^{- 1} {cm}^{- 1} ) of disitamab vedotin ;$$

$${A}_{280} = the average absorbance of the sample solution at 280 nm ;$$

### System Suitability

1) the CV values of absorbance values were measured in parallel≤2.0%
2) the Blank absorbance value A280≤0.001

### Standard Limit

The protein concentration of sample is 9.0˜11.0 mg/ml.

### Example 5: Interim Results of a Phase I Clinical Study to Evaluate the Safety, Tolerability, Pharmacokinetic Characteristics, and Preliminary Efficacy of RC48-ADC in Combination with Toripalimab for the Treatment of Patients with Locally Advanced or Metastatic Solid Tumors Expressing HER2

Antibody-drug conjugate (ADC) significantly improved objective response rates and survival outcomes in patients with solid tumors such as HER2-positive or low-expressing breast cancer. Preclinical data suggest that RC-48 and PD-1 inhibitors have synergistic antitumor effects. To evaluate the safety and efficacy of RC48 in combination with the PD-1 antibody toripalimab in patients with HER2-expressing metastatic breast cancer and other solid tumors, a multi-center, Phase I clinical trial (NCT04280341) was conducted. The current Example describes the results of the clinical trial, the detailed protocol of which is described in Example 1.

In brief, the study design is as follows:
Patients who have failed first-line or standard treatment (disease progression after treatment or intolerable treatment toxicity) or have no effective treatment options.
The definition of HER2 expression is as follows (meeting one of the following):

$$HER 2 IHC 3 + , 2 + , 1 +$$

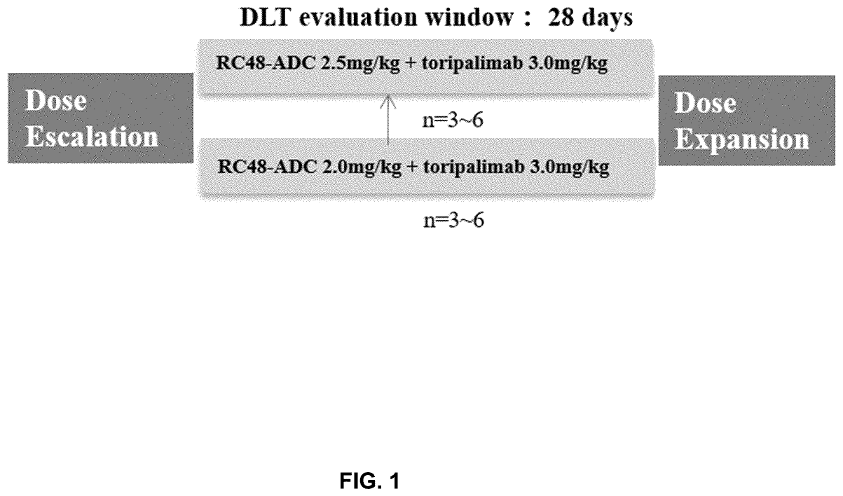

HER2 gene amplification (FISH+: HER2/CEP17 ratio >2), if the subject has breast cancer, the criteria for breast cancer gene amplification are referred to)
The dosing interval is Q2W (every two weeks), until disease progression, death, intolerable toxicity, or reaching other criteria for study treatment discontinuation as stipulated by the protocol. The dose limiting toxicity (DLT) evaluation window is 28 days. This study consists of two phases of dose escalation and dose expansion (see, FIG. 1).

Efficacy Evaluation: Tumor imaging assessments will be conducted once every 6 weeks (+7 days) starting from the first administration of the drug, and after 48 weeks, the assessments will be conducted once every 12 weeks (+7 days), until disease progression, death, intolerable toxicity, or reaching other criteria for study treatment discontinuation as stipulated by the protocol, whichever occurs first.

### Interim Results: Efficacy Data Analysis

In the breast cancer population of the RC48-C013 study, the efficacy and safety of RC48-ADC (2.5 mg/kg, Q2W) combined with toripalimab (3 mg/kg, Q2W) for the treatment of metastatic breast cancer were explored.

In the treatment of HR+HER2-low expressing breast cancer that had received at least one line of chemotherapy with RC48-ADC (2.5 mg/kg, Q2W) combined with toripalimab (3 mg/kg, Q2W), a total of 7 cases were enrolled, with 5 cases being endocrine-resistant subjects. The confirmed ORR and median PFS of the combined therapy were 42.9% and 9.6 months (95% CI: 1.4, NE), respectively.

In the treatment of HR+HER2-low expressing breast cancer that had received at least one line of chemotherapy with RC48-ADC (2.0 mg/kg, Q2W) combined with toripalimab (3 mg/kg, Q2W), a total of 7 cases were enrolled, and the confirmed ORR and median PFS were 42.9% and 4.21 months (95% CI: 2.6, NE), respectively.

In the treatment of HR-HER2-low expressing breast cancer that had received at least one line of chemotherapy with RC48-ADC (2.0 mg/kg, Q2W) combined with toripalimab (3 mg/kg, Q2W), a total of 4 cases were enrolled. As of the data cutoff, 3 cases had not reached a PFS event. To date, there has been 1 confirmed CR, 1 confirmed PR, 1 SD, and 1 PD.

### Interim Results: Safety Data Analysis

In the RC48-C013 study, at a dosage of 2.0 mg/kg, 8 breast cancer patients were available for safety analysis, with insufficient exposure, the median exposure time was (RC48: 13.2 weeks, JS001: 8.9 weeks). At a dosage of 2.5 mg/kg, there were 14 breast cancer patients available for safety analysis, with a median exposure time (RC48: 17.8 weeks, JS001: 15.5 weeks).

Ninety-five percent of patients in this study experienced at least one TEAE (Treatment-Emergent Adverse Event), with the most common AE (Adverse Event) being abnormal liver function tests, mainly increased AST (64%) and ALT (59%), all classified as CTCAE grades 1-2. One case of grade 3 liver function abnormality occurred in the 2.0 mg/kg dose group.

Common AEs included hematological abnormalities, mainly a decrease in white blood cell count and neutrophil count, both above 50%. Other common AEs included fatigue, hypoesthesia, alopecia, decreased appetite, pruritus, and nausea.

Overall, the incidence rate in the 2.0 mg/kg dose group was lower than that in the 2.5 mg/kg dose group. Compared with the previous safety data of RC48, the incidence of pruritus, abdominal pain, and abdominal distension in the 2.0 mg/kg dose group increased, while other AEs were comparable to or lower than those of monotherapy with RC48. Due to the limited sample size of the 2.0 mg/kg dose group, the safety assessment based on this data is not yet sufficient.

There were 5 patients with drug-related SAEs (Serious Adverse Events), 4 of whom were in the 2.5 mg/kg dose group, with 2 patients experiencing immune-related pneumonitis, and other SAEs including pulmonary inflammation, decreased white blood cell count, diabetic ketoacidosis, decreased neutrophil count, and sudden death. In the 2.0 mg/kg dose group, 1 patient had an SAE of abnormal liver function.

### Example 6: a Randomized, Open-Label, Multicenter Phase II/III Clinical Study Evaluating the Efficacy and Safety of RC48-ADC in Combination with Toripalimab and Chemotherapy or RC48-ADC in Combination with Toripalimab and Trastuzumab in First-Line Treatment of Patients with HER2-Expressing Locally Advanced or Metastatic Gastric Cancer (Including Adenocarcinoma of the Gastroesophageal Junction)

This example describes a phase II/III clinical study evaluating the efficacy and safety of RC48-ADC in combination with toripalimab and chemotherapy or RC48-ADC in combination with toripalimab and trastuzumab in first-line treatment of patients with HER2-expressing locally advanced or metastatic gastric cancer (including adenocarcinoma of the gastroesophageal junction).

A total of 170-201 patients are planned to be enrolled. Interim analysis of study results from 109 subjects is described in Example 7.

### Design

This clinical study is a randomized, open-label, parallel controlled, multicenter clinical study with PD-L1 Combined Positive Score (CPS)>1 or combined positive score <1, HER2-expressing locally advanced or metastatic gastric cancer (including adenocarcinoma of the gastroesophageal junction) who have not previously received systemic chemotherapy for locally advanced or metastatic gastric cancer (including adenocarcinoma of the gastroesophageal junction). The clinical study is mainly divided into two parts:

Part I: Phase II study phase

The Phase II study is designed to evaluate the preliminary validity of three cohorts and four combination treatment regimens, and eligible subjects in each cohort are randomly assigned to the trial or control group based on CPS level (CPS ≥1 or CPS <1). The cohorts and treatment groups are as follows:

### 1) Cohort 1 (HER2 Positive):

Cohort 1 set up three dosing regimens, subjects are randomly assigned to one of the following regiments in 1:1:1 for study dosing, a total of about 51 subjects are enrolled.
   Trial group 1: RC48-ADC+toripalimab+Chemotherapy (CAPOX: Capecitabine+Oxaliplatin):

The overall regimen of combined dosing every 6 weeks (42 days) as a treatment cycle; The timing of the first dosing (if simultaneous dosing occurs on the same day) is as follows: RC48-ADC-toripalimab-oxaliplatin-capecitabine (the infusion time of RC48-ADC is 30-90 minutes (recommend 60 minutes), the first intravenous infusion of toripalimab is completed in about 60 minutes, if the first infusion is well tolerated, the second infusion time can be shortened to 30 minutes. The interval between the two medications is 30-60 minutes; Intravenous oxaliplatin infusion is started at least 60 minutes after the completion of the toripalimab infusion, and the intravenous infusion of oxaliplatin should be completed within 3-4 hours. Capecitabine should be taken orally once on the first day of the night, twice a day from the second day to the 14th day, and the interval between two oral dosing is recommended within 10-12 hours, i.e., once in the morning and once in the evening, and once in the morning on the 15th day).

Specific dosing are as follows:
RC48-ADC: 2.5 mg/kg, intravenous infusion, Q2W;
Toripalimab: 3 mg/kg, intravenous infusion, Q2W;
CAPOX: Oxaliplatin: 100 mg/m², intravenous infusion, dayl, Q3W; Capecitabine:

750 mg/m², taken orally, day 1-14, Q3W.

Trial group 2: RC48-ADC+toripalimab+trastuzumab:

The overall regimen of combination dosing was every 6 weeks (42 days) as a treatment cycle; Dosing timing (if dosing is simultaneous on the same day): RC48-ADC-toripalimab-trastuzumab (the infusion time of RC48-ADC is 30-90 minutes (recommend 60 minutes), the first intravenous infusion of toripalimab is completed in about 60 minutes, if the first infusion is well tolerated, the second infusion time can be shortened to 30 minutes. The interval between the two medications is 30-60 minutes; Intravenous infusion of trastuzumab is started within 60 minutes of the completion of toripalimab infusion, and the first infusion time is about 90 minutes, which can be changed to 30 minutes later if the patient is well tolerated at the first infusion.

Specific dosing are as follows:
RC48-ADC: 2.5 mg/kg, intravenous infusion, Q2W;
Toripalimab: 3 mg/kg, intravenous infusion, Q2W;
Trastuzumab: Initial load dose of 8 mg/kg, intravenous infusion, followed by 6 mg/kg, Q3W.
   Control group: toripalimab+trastuzumab+CAPOX:
The overall regimen of combination dosing was every 6 weeks (42 days) as a treatment cycle; Dosing timing (if dosing is simultaneous on the same day): toripalimab-trastuzumab-CAPOX (intravenous infusion of trastuzumab is started within 60 minutes after the completion of toripalimab infusion, the first infusion time is about 90 minutes, if the patient is well tolerated at the first infusion, the subsequent can be changed to 30 minutes, and oxaliplatin intravenous infusion is started within 60 minutes after the completion of trastuzumab infusion. Intravenous infusion is completed within 3-4 hours; Capecitabine should be taken orally once on the first day of the night, twice a day from the second day to the 14th day, and the interval between two oral dosing is recommended within 10-12 hours, i.e., once in the morning and once in the evening, and once in the morning on the 15th day).

Specific dosing are as follows:
Toripalimab: 3 mg/kg, intravenous infusion, Q2W;
Trastuzumab: Initial load dose of 8 mg/kg, intravenous infusion, followed by 6 mg/kg, Q3W;
CAPOX: Oxaliplatin: 130 mg/m², intravenous infusion, day 1, Q3W; Capecitabine: 1000 mg/m², take orally, day 1-14, Q3W.

### 2) Cohort 2 (HER2-Low):

Cohort 2 set up three dosing regimens, subjects are randomly assigned to one of the following regiments in 1:1 for study dosing, a total of 90-105 subjects are enrolled (including 30-45 expect to be enrolled under the current regimen and about 60 enrolled under the previous regimen).

### Trial Group 1: RC48-ADC+Toripalimab+CAPOX:

The overall regimen of combined administration every 6 weeks (42 days) as a treatment cycle; First dosing sequence (if simultaneous dosing on the same day): RC48-ADC-toripalimab-oxaliplatin-capecitabine (dosing interval and dosing time are the same as that of trial group 1 in the Cohort 1).

Specific dosing are as follows:
RC48-ADC: 2.5 mg/kg, intravenous infusion, Q2W;
Toripalimab: 3 mg/kg, intravenous infusion, Q2W;
CAPOX: Oxaliplatin: 100 mg/m², intravenous infusion, day 1, Q3W; Capecitabine: 750 mg/m², take orally, day 1-14, Q3W.

### Trial Group 2: RC48-ADC+Toripalimab+CAPOX:

The overall regimen of combined administration every 6 weeks (42 days) as a treatment cycle; First dosing sequence (if simultaneous dosing on the same day): RC48-ADC-toripalimab-oxaliplatin-capecitabine (dosing interval and dosing time are the same as that of trial group 1 in the Cohort 1).

Specific dosing are as follows:
RC48-ADC: 2.0 mg/kg, intravenous infusion, Q2W;
Toripalimab: 3 mg/kg, intravenous infusion, Q2W;
CAPOX: Oxaliplatin: 100 mg/m², intravenous infusion, day 1, Q3W; Capecitabine: 750 mg/m², take orally, day 1-14, Q3W.

### Control Group: Toripalimab+CAPOX:

The overall regimen of combined administration every 6 weeks (42 days) as a treatment cycle; First dosing sequence (if simultaneous dosing on the same day): toripalimab-oxaliplatin-capecitabine (intravenous infusion of oxaliplatin is started within 60 minutes after the completion of toripalimab infusion, and oxaliplatin intravenous infusion can be completed within 3-4 hours; Capecitabine should be taken orally once on the first day of the night, twice a day from the second day to the 14th day, and the interval between two oral dosing is recommended within 10-12 hours, i.e., once in the morning and once in the evening, and once in the morning on the 15th day).

Specific dosing are as follows:
Toripalimab: 3 mg/kg, intravenous infusion, Q2W;
CAPOX: Oxaliplatin: 130 mg/m², intravenous infusion, dayl, Q3W; Capecitabine: 1000 mg/m², take orally, day 1-14, Q3W.

### 3) Cohort 3 HER2-0 (IHC 0):

Cohort 3 set up three dosing regimens, subjects are randomly assigned to one of the following regiments in 1:1 for study dosing, a total of 30-45 subjects are enrolled.

### Trial Group 1: RC48-ADC+Toripalimab+CAPOX:

The overall regimen of combined administration every 6 weeks (42 days) as a treatment cycle; First dosing sequence (if simultaneous dosing on the same day): RC48-ADC-toripalimab-oxaliplatin-capecitabine (dosing interval and dosing time are the same as that of trial group 1 in the Cohort 1).

Specific dosing are as follows:
RC48-ADC: 2.5 mg/kg, intravenous infusion, Q2W;
Toripalimab: 3 mg/kg, intravenous infusion, Q2W;
CAPOX: Oxaliplatin: 100 mg/m², intravenous infusion, day 1, Q3W; Capecitabine: 750 mg/m², take orally, day 1-14, Q3W.

### Trial Group 2: RC48-ADC+Toripalimab+CAPOX:

The overall regimen of combined administration every 6 weeks (42 days) as a treatment cycle; First dosing sequence (if simultaneous dosing on the same day): RC48-ADC-toripalimab-oxaliplatin-capecitabine (dosing interval and dosing time are the same as that of trial group 1 in the Cohort 1).

Specific dosing are as follows:
RC48-ADC: 2.0 mg/kg, intravenous infusion, Q2W;
Toripalimab: 3 mg/kg, intravenous infusion, Q2W;
CAPOX: Oxaliplatin: 100 mg/m², intravenous infusion, day 1, Q3W; Capecitabine: 750 mg/m², take orally, day 1-14, Q3W.

### Control Group: Toripalimab+CAPOX:

The overall regimen of combined administration every 6 weeks (42 days) as a treatment cycle; First dosing sequence (if simultaneous dosing on the same day): toripalimab-oxaliplatin-capecitabine (intravenous infusion of oxaliplatin is started within 60 minutes after the completion of toripalimab infusion, and oxaliplatin intravenous infusion can be completed within 3-4 hours; Capecitabine should be taken orally once on the first day of the night, twice a day from the second day to the 14th day, and the interval between two oral dosing is recommended within 10-12 hours, i.e., once in the morning and once in the evening, and once in the morning on the 15th day).

Specific dosing are as follows:
Toripalimab: 3 mg/kg, intravenous infusion, Q2W;
CAPOX: Oxaliplatin: 130 mg/m², intravenous infusion, day 1, Q3W; Capecitabine: 1000 mg/m², take orally, day 1-14, Q3W.

Participants in these three cohorts continued medication until intolerable toxicity, progressive disease, initiation of a new antitumor therapy, loss of follow-up, withdrawal of informed consent, or death, or sponsor's decision to terminate the study. Among them, Oxaliplatin was only administered in the first 3 treatment cycles for 6 times of Q3W study.

To evaluate the safety of participants during the study while collect the adverse events and drug combinations. For drug-related SAE, follow-up until recovery, remission, or no longer changed by the investigator. Participants baseline in this study must have measurable lesions as defined by the RECIST v1.1 criteria, and tumor evaluations are performed every 6 weeks (+7 days) during the study until progressive disease, loss of follow-up, withdrawal of informed consent, or death. The tumor evaluation time was not affected by dose adjustment or interruption. Evaluating the efficacy of the subjects' tumors, and after treatment, all subjects will have survival follow-up every 3 months until death, loss of follow-up, withdrawal of informed consent, or sponsor's decision to terminate the study.

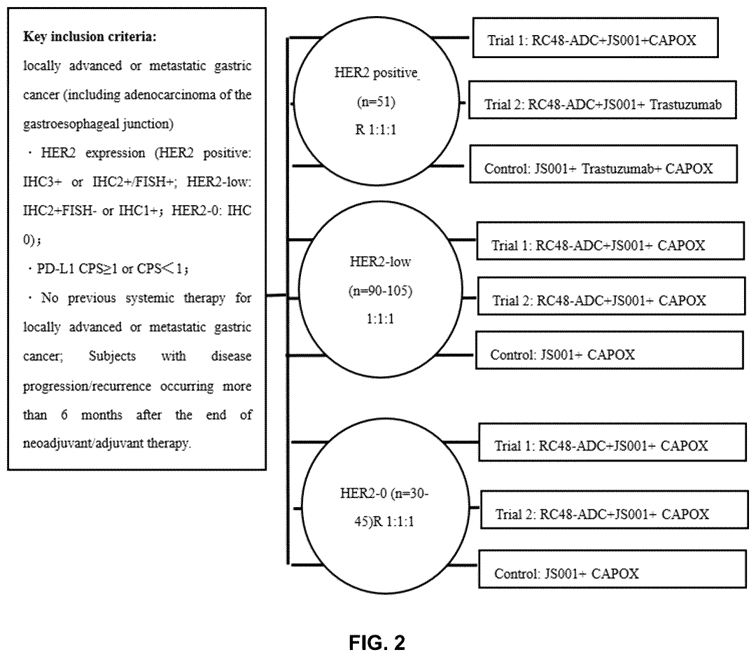

The overall design of the clinical study is shown in FIG. 2.

Based on the comprehensive consideration of the safety and efficacy clinical data of the Phase II exploratory study, further adjustments are made to the dosing schedule, dosage, frequency and cycle of dosing of the Phase III.

### Part II: Phase III Study Phase

The Phase III study phase will set up a central laboratory to test HER2 immunohistochemistry and PD-L1 in all subjects who signed informed consent.

The clinical study divides two study cohorts: Cohort1 (HER2+) and Cohort2 (HER2-low). Eligible subjects in each cohort were selected according to CPS level (CPS ≥5 or CPS <5) stratified and randomly assigned to trial group or control group in 1:1.

Settings are as follows:

### 1) Cohort 1 (HER2 Positive):

### Trial Group: RC48-ADC+Toripalimab+CAPOX OR RC48-ADC+Toripalimab+Trastuzumab

### \textcircled{1} RC48-ADC+Toripalimab+CAPOX:

The overall regimen of combined administration every 6 weeks (42 days) as a treatment cycle; First dosing sequence (if simultaneous dosing on the same day): RC48-ADC-toripalimab-oxaliplatin-capecitabine, the recommended dosing interval is no more than 30 minutes or as determined by the investigator.

Specific dosing are as follows:
RC48-ADC: 2.5 mg/kg, intravenous infusion, Q2W;
Toripalimab: 3 mg/kg, intravenous infusion, Q2W;
CAPOX: Oxaliplatin: 100 mg/m², intravenous infusion, dayl, Q3W; Capecitabine: 1000 mg/m², take orally, day 1-14, Q3W.

### \textcircled{2} RC48-ADC+Toripalimab+Trastuzumab:

The overall regimen of combined administration every 6 weeks (42 days) as a treatment cycle; Dosing sequence (if simultaneous dosing on the same day): trastuzumab-RC48-ADC-toripalimab, the recommended dosing interval is no more than 30 minutes or as determined by the investigator.

Specific dosing are as follows:
RC48-ADC: 2.5 mg/kg, intravenous infusion, Q2W;
Toripalimab: 3 mg/kg, intravenous infusion, Q2W;
Trastuzumab: Initial load dose of 8 mg/kg, intravenous infusion, followed by 6 mg/kg,
Q3W.

### Control Group: Trastuzumab+CAPOX

The dosing regimen was every 6 weeks (42 days) as a treatment cycle.

Specific dosing are as follows:
Trastuzumab: Initial load dose of 8 mg/kg, intravenous infusion, followed by 6 mg/kg, Q3W;
CAPOX: Oxaliplatin: 130 mg/m², intravenous infusion, day 1, Q3W; Capecitabine: 1000 mg/m², take orally, day 1-14, Q3W.

### 2) Cohort 2 (HER2-Low):

### Trial Group: RC48-ADC+Toripalimab+CAPOX

The dosing regimen was every 6 weeks (42 days) as a treatment cycle; Dosing sequence (if simultaneous dosing on the same day): RC48-ADC-toripalimab-Oxaliplatin-Capecitabine, the recommended dosing interval is no more than 30 minutes or as determined by the investigator.

Specific dosing are as follows:
RC48-ADC: 2.5 mg/kg, intravenous infusion, Q2W;
Toripalimab: 3 mg/kg, intravenous infusion, Q2W;
CAPOX: Oxaliplatin: 100 mg/m², intravenous infusion, dayl, Q3W; Capecitabine: 1000 mg/m², take orally, day 1-14, Q3W.

### Control Group: CAPOX

The dosing regimen was every 6 weeks (42 days) as a treatment cycle; Dosing sequence: Oxaliplatin-Capecitabine, the recommended dosing interval is no more than 30 minutes or as determined by the investigator.

Specific dosing are as follows:
CAPOX: Oxaliplatin: 100 mg/m², intravenous infusion, day 1, Q3W; Capecitabine: 1000 mg/m², take orally, day 1-14, Q3W.

After the dose and the dosing regimen in above two cohorts is 4-6 cycles of maintenance therapy (or at the discretion of the investigator), allow the choice of options based on the clinician's judgment and the patient's actual situation to ensure maximum clinical benefit for the subjects.

To evaluate the safety of participants during the study while collect the adverse events and drug combinations. For drug-related SAE, follow-up until recovery, remission, or no longer changed by the investigator. Participants baseline in this study must have measurable lesions as defined by the RECIST v1.1 criteria, and tumor evaluations are performed every 6 weeks (+7 days) during the study until progressive disease, loss of follow-up, withdrawal of informed consent, or death. The tumor evaluation time was not affected by dose adjustment or interruption.

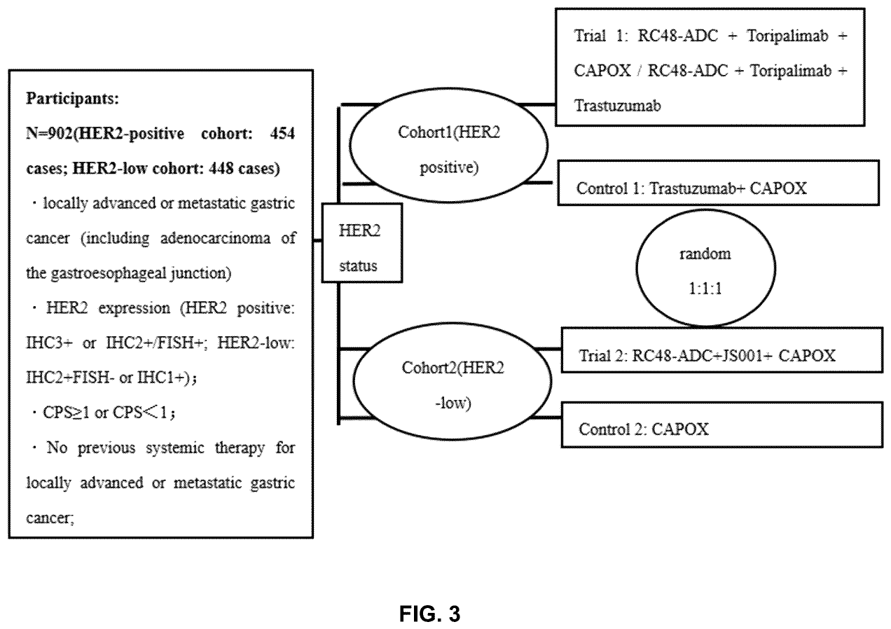

The overall design of the clinical study is shown in FIG. 3.

PK analysis and ADA analysis were performed in the study. During the study, the experimental group used a sparse PK blood collection design to detect the concentration of RC48-ADC total antibody, conjugated antibody, free MMAE, and trastuzumab for injection in blood samples, and analyze the immunogenicity of RC48-ADC.

### Objectives

### Part I: Phase II Study Phase

The primary objective of the study is to evaluate the preliminary validity of RC48-ADC in combination with toripalimab and chemotherapy or RC48-ADC in combination with toripalimab and trastuzumab in first-line treatment of patients with HER2-expressing locally advanced or metastatic gastric cancer (including adenocarcinoma of the gastroesophageal junction). The primary endpoint is objective response rate (ORR) (CPS ≥1, CPS ≥5, overall population); (investigator evaluation).

Secondary objectives of the study include:
To evaluate the safety of RC48-ADC in combination with toripalimab and chemotherapy or RC48-ADC in combination with toripalimab and trastuzumab in first-line treatment of patients with HER2-expressing locally advanced or metastatic gastric cancer (including adenocarcinoma of the gastroesophageal junction);
To evaluate the efficacy of RC48-ADC in combination with toripalimab and chemotherapy or RC48-ADC in combination with toripalimab and trastuzumab in first-line treatment of patients with HER2-expressing locally advanced or metastatic gastric cancer (including adenocarcinoma of the gastroesophageal junction);
To evaluate the pharmacokinetic characteristics of RC48-ADC in combination with toripalimab and chemotherapy or RC48-ADC in combination with toripalimab and trastuzumab in first-line treatment of patients with HER2-expressing locally advanced or metastatic gastric cancer (including adenocarcinoma of the gastroesophageal junction);
To evaluate the immunogenicity of RC48-ADC in combination with toripalimab and chemotherapy or RC48-ADC in combination with toripalimab and trastuzumab in first-line treatment of patients with HER2-expressing locally advanced or metastatic gastric cancer (including adenocarcinoma of the gastroesophageal junction).

Secondary safety endpoints of the study include:
Adverse events (AE);
Vital signs, physical examination, laboratory examination, electrocardiogram, echocardiography

Secondary efficacy endpoints of the study include:
Overall survival (OS) (CPS ≥1, CPS ≥5, overall population);
Progression-free survival (PFS) (CPS ≥1, CPS ≥5, overall population) (investigator evaluation);
Duration of response (DOR) (CPS ≥1, CPS ≥5, overall population) (investigator evaluation);
Disease control rate (DCR) (CPS ≥1, CPS ≥5, overall population) (investigator evaluation).

Secondary pharmacokinetic endpoints of the study include:
Peak and valley concentrations of total antibody, binding antibody and free MMAE of RC48-ADC.
Peak and valley concentrations of trastuzumab.

Secondary immunogenicity endpoints of the study include:
Positive incidence, time of occurrence, duration, and titer of anti-drug and/or neutralizing antibodies of against disitamab vedotin for injection.

### Part II: Phase III Study Phase

The primary objective of this study is to evaluate the efficacy of RC48-ADC in combination with toripalimab and chemotherapy or RC48-ADC in combination with toripalimab and trastuzumab in first-line treatment of patients with HER2-expressing locally advanced or metastatic gastric cancer (including adenocarcinoma of the gastroesophageal junction). The primary endpoint of the study is progression-free survival (PFS, CPS ≥5) (IRC assessment).

Secondary objectives of the study include:
To evaluate the safety of RC48-ADC in combination with toripalimab and chemotherapy or RC48-ADC in combination with toripalimab and trastuzumab in first-line treatment of patients with HER2-expressing locally advanced or metastatic gastric cancer (including adenocarcinoma of the gastroesophageal junction);
To evaluate the pharmacokinetic characteristics and immunogenicity of RC48-ADC in combination with toripalimab and chemotherapy or RC48-ADC in combination with toripalimab and trastuzumab in first-line treatment of patients with HER2-expressing locally advanced or metastatic gastric cancer (including adenocarcinoma of the gastroesophageal junction).

Secondary efficacy endpoints of the study include:
Objective response rate (ORR) (CPS ≥1, CPS ≥5, overall population); (investigator evaluation);
Overall survival (OS) (CPS ≥1, CPS ≥5, overall population);
Progression-free survival (PFS) (CPS ≥1, CPS ≥5, overall population) (investigator evaluation);
Duration of response (DOR) (CPS ≥1, CPS ≥5, overall population) (investigator evaluation);
Disease control rate (DCR) (CPS ≥1, CPS ≥5, overall population) (investigator evaluation).

Secondary safety endpoints of the study include:
Adverse events (AE);
Vital signs, physical examination, laboratory examination, electrocardiogram, echocardiography.

Secondary pharmacokinetic endpoints of the study include:
Pharmacokinetic of total antibody, binding antibody and free MMAE of RC48-ADC.

Secondary immunogenicity endpoints of the study include:
Positive incidence, time of occurrence, duration, and titer of anti-drug and/or neutralizing antibodies against disitamab vedotin for injection.

### Participants

HER2 expression or HER2 non-expression (HER2 expression includes: HER2-positive: IHC3+ or IHC2+/FISH+ and HER2− low expression: IHC2+/FISH− or IHC1+; HER2 is not expressed: HER2-0: IHC 0) locally advanced or metastatic gastric cancer (including adenocarcinoma of the gastroesophageal junction) and PD-L1 CPS ≥1 or CPS <1.

### Inclusion Criteria

Inclusion criteria for enrollment in the study are as follows:

### General Condition

1. Voluntarily participate and sign the informed consent form;

2. Ages 18-75 (including 18 and 75);

3. Expected survival ≥12 weeks;

4. Having an ECOG performance status score of 0 or 1;

5. Female subjects should be surgically sterilized, postmenopausal, or agree to use at least one medically acceptable method of contraception (e.g., intrauterine device, contraceptives, or condoms) during study treatment and for 6 months after the end of the study treatment period, and must have a negative blood pregnancy test or false positive blood pregnancy tests excluded by the investigator within 7 days prior to study entry, and must be non-lactating. Male subjects should agree to use at least one medically approved contraceptive during the treatment and within 6 months after the treatment;
6. Willing and able to comply with trial and follow-up procedures;

### Adequate Organ Function

7. Bone marrow function:
   Hemoglobin ≥9 g/dL;
   Absolute neutrophil count (ANC) ≥1.5×10⁹/L;
   Platelet ≥100×109/L;
8. Liver function (based on the normal value of the clinical trial center):
   Serum total bilirubin≤1.5 times the upper limit of normal (ULN);
   Without liver metastasis: ALT and AST≤2.5×ULN, with liver metastases: ALT and AST≤5×ULN;
9. Renal function (based on the normal value of the clinical trial center):
   Serum creatinine≤1.5×ULN or creatinine clearance (CrCl) ≥50 mL/min according to Cockcroft-Gault formula or 24-hour urine CrCl≥60 rmL/min;
10. Coagulation function: International Normalized ratio (INR), activated partial thromboplastin time (APTT) and prothrombin time (PT) are all≤1.5 times ULN;
11. Heart function:
   NYHA cardiac function grading<3;
   Left ventricular ejection fraction ≥50%;
   Screening period QTcF interval≤470 ms (based on the average of 3 screening electrocardiograms);

### Tumor Related

12. Histologically and/or cytologically confirmed locally advanced or metastatic gastric cancer that is inoperable (including adenocarcinoma of the gastroesophageal junction);
13. Had not previously received systemic chemotherapy for locally advanced or metastatic gastric cancer; No progressive disease/recurrence for more than 6 months after the end of neoadjuvant/adjuvant therapy;
14. Confirm the expression status of PD-L1 (CPS≥1 or CPS<1);
15. Confirm the expression status of HER2: IHC 1+, 2+ (to determine FISH status) or 3+ or IHC 0;
16. There is at least one measurable lesion according to RECISTv1.1 standard.

### Exclusion Criteria

Subjects who meet any of the following criteria must be excluded from this study plan:
1. Central nervous system (CNS) metastases and/or carcinomatous meningitis and subjects who have undergone treatment for brain metastases may be eligible, provided that the subject has been in stable condition for at least 3 months, no radiographic evidence of progressive disease within 4 weeks prior to the first dose, all neurological symptoms have returned to baseline levels, and no new or growth of brain metastasis, and the subject has discontinued radiation, surgery or steroid therapy at least 28 days prior to the first dose. This does not include subjects with carcinomatous meningitis, who are not eligible even if clinically stable;
2. Toxicity from previous antitumor therapy has not returned to CTCAE (version 5.0) level 0-1 (except for alopecia, pigmentation, or other conditions that the investigator believes do not increase the subject's risk of medication);
3. History of major surgery that has not fully recovered within 4 weeks prior to dosing;
4. Received live vaccine within 4 weeks prior to dose administration or plan on receiving any vaccine during the study (except for Vaccination against COVID-19);
5. Pleural effusion and ascites with symptoms or requiring symptomatic treatment;
6. Serum virology (based on normal reference ranges of the clinical trial site):
   Subjects with positive HBsAg and positive HBV DNA copy number;
   Positive HCVAb (only subject who has a negative HCV RNA in PCR test will be eligible);
   Positive HIVAb.
7. Serious arterial/venous thrombotic events or cardiovascular and cerebrovascular accidents within 1 year prior to dose administration, such as deep venous thrombosis (excluding asymptomatic intermuscular venous thrombosis not requiring special treatment), pulmonary embolism, cerebral infarction, cerebral hemorrhage, myocardial infarction, etc., except for asymptomatic lacunar infarction not requiring clinical intervention;
8. Tumor lesions have tendency to bleed (such as active ulcer tumor lesions and positive fecal occult blood test, history of hematemesis or black stool within 2 months before signing the informed consent, and risk of massive gastrointestinal bleeding by the investigator) or have received blood transfusion treatment 4 weeks before dosing;
9. Active or progressive infection requiring systemic treatment, such as active tuberculosis;
10. Subjects who have uncontrolled systemic diseases, including diabetes mellitus, hypertension, cirrhosis, pneumonia, obstructive pulmonary disease, etc., as judged by the investigator;
11. Active autoimmune diseases requiring systemic therapy (such as the use of disease-modifying drugs, corticosteroids, or immunosuppressive drugs) within 2 years prior to dose administration, and replacement therapies (e.g., thyroxine, insulin, or physiological replacement of glucocorticoids due to renal or pituitary deficiency) are allowed;
12. History of other malignant tumors within 5 years prior to dose administration, but except for:
   Malignancies that can be cured after treatment (including but not limited to adequately treated thyroid cancer, cervical carcinoma in situ, basal or squamous cell skin cancer, or radical treatment of ductal carcinoma in situ of the breast);
13. Received radiotherapy or Chinese medicine treatment within 2 weeks (Chinese medicine treatment with clear anti-tumor indications in the instructions);
14. Received allogeneic hematopoietic stem cell transplantation;
15. Previous treatment with a PD-(L) 1 inhibitor or any other ADC drugs;
16. Known allergy to PD-(L) 1 inhibitors and any other ADC drugs and their components;
17. Pregnant or lactating women;
18. Any other disease, metabolic disorder, or abnormal findings upon physical examination or laboratory examination that makes the subject unsuitable for receiving the investigational drug, affects the interpretation of study outcomes, or poses risks to patient safety, as determined by the investigator;
19. Subject is assessed to have insufficient adherence to this clinical study, or subjects who have other factors that the investigator deems unsuitable for participation in the study.

### Sample Size Calculation

### Part I: Phase II Study Phase

171-201 participants are planned to be enrolled in this phase.

There are a total of about 51 patients in cohort 1 (about 17 in each of the three treatment groups), 90-105 patients in cohort 2 (including 30-45 patients expected to be enrolled under the current regimen (10-15 in each of the three treatment groups), and about 60 patients in cohort 3 (10-15 in each of the three treatment groups), and 30-45 patients in cohort 3 (10-15 in each of the three treatment groups).

### Part II: Phase III Study Phase

The primary endpoint of the study was PFS (IRC assessment) in patients with CPS ≥5. Based on the following parameter assumptions, R calculated that at least 540 patients with CPS ≥5 were enrolled in this study, patients with CPS ≥5 are expected to account for approximately 60% of the total patients, this results in the planned enrollment of 902 subjects (454 in Cohort 1, 227 in each group and 448 in Cohort 2, 224 in each group). The sample size of this stage will refer to the efficacy results of the Phase II study phase.

Cohort 1 (HER2 positive): According to a previous published papers (see, Janjigian Y Y, Ajani J A, Moehler M, et al. Ann Oncol, 2021, 32 (5): S1283-S1346; Zhang X, Wang J, Wang G, et al. Annals of Oncology, 2023, 34: S1319; Janjigian Y Y, Kawazoe A, Bai Y, et al. Annals of Oncology, 2023, 34: S851-S852; Rha S Y, Wyrwicz L, Yanez Weber P E, et al. 2023; Xu J, Jiang H, Pan Y, et al. Annals of Oncology, 2021, 32: S1331; Moehler M H, Kato K, Arkenau H T, et al. 2023), the median PFS (IRC) of the CPS ≥5 patients control group was 8 months, assuming that the HR of the PFS (IRC) in the trial group and the control group was 0.65, the enrollment period was 48 months, and the follow-up period after enrollment of the last patient was 20 months, with an annual drop-out rate of about 10%. Alpha was 0.05 on both sides and the power was 90%. Using Log-rank test, participants with Cohort 1 CPS ≥5 had to observe 227 PFS endpoint events and 272 participants are expected to be enrolled (1:1 enrollment, 136 in each group).

Cohort 2 (HER2-low): According to previous published paper (see, Janjigian Y Y, Ajani J A, Moehler M, et al. Ann Oncol, 2021, 32 (5): S1283-S1346; Zhang X, Wang J, Wang G, et al. Annals of Oncology, 2023, 34: S1319; Janjigian Y Y, Kawazoe A, Bai Y, et al. Annals of Oncology, 2023, 34: S851-S852; Rha S Y, Wyrwicz L, Yanez Weber P E, et al. 2023; Xu J, Jiang H, Pan Y, et al. Annals of Oncology, 2021, 32: S1331; Moehler M H, Kato K, Arkenau H T, et al. 2023), the median PFS (IRC) of the CPS ≥5 patients control group was 7.5 months, assuming that the HR of the PFS in the trial group and the control group was 0.65, other parameters are the same as Cohort 1. Using Log-rank test, participants with Cohort 2 CPS ≥5 had to observe 227 PFS endpoint events and 268 participants are expected to be enrolled (1:1 enrollment, 134 in each group).

### Statistical Analysis

Statistical analysis refers to the Statistical Analysis Plan (SAP) of the study. Before the database is locked, statisticians will discuss with the main researchers and sponsors, and finalize according to the data characteristics. This plan only describes the basic statistical analysis content.

In this study, statistical analysis considering the end point of survival was conducted separately after the completion of the corresponding part of the follow-up, and the statistical analysis report was submitted in stages.

Unless otherwise noted, all statistical tests in the study were bilateral, with an overall Class I error level of 0.05. Unless special instructions, descriptive statistics of continuous variables will mainly include case number, mean, standard deviation, median, minimum and maximum, and categorical variables perform descriptive statistics using frequency and percentage. Event-time variables using Kaplan-Meier method to calculate the median and quartile of each group and their 95% confidence intervals, and to draw survival curves. At the same time, event incidence and deletion rate at the end of the study were provided. Comparisons between treatment groups used a log-rank test that corrected for random stratified factors, along with HR estimates and confidence intervals for the COX proportional hazards regression model.

### Part I: Phase II Study Phase

In the trial phase, descriptive statistical analysis such as safety, efficacy and PK were performed according to the treatment plan.

### Part II: Phase III Study Phase

The trial phase is the main scheme design, and the two target populations (Cohort) are independent from each other and do not share a. Statistical tests were conducted at the bilateral test level of 0.05 respectively. Within each Cohort there was one primary endpoint IRC assessment of PFS (IRC, CPS≥5), another part of the secondary endpoints OS (CPS ≥5, CPS≥1, overall population) and PFS (IRC, CPS ≥1, overall population), ORR (IRC, CPS ≥5)) Statistical tests were performed. In order to control their overall Class 1 error rates, the primary endpoint PFS (IRC, CPS ≥5) were tested at the bilateral 0.05 test level, and the secondary endpoint were statistically tested if and only if the invalid hypothesis of the primary endpoint PFS (IRC, CPS ≥5) was rejected, and all unused a were passed to the secondary endpoint. Secondary endpoints were tested in a preset fixed order, only when the analysis results of one secondary endpoint supported the rejection of the invalid hypothesis can the next endpoint be tested statistically. In addition, the invalidity interim analysis of each cohort was analyzed as non-binding, so it did not affect the overall Class 1 error.

### Example 7: Interim Analysis of Phase III Clinical Study Evaluating the Efficacy and Safety of RC48-ADC in Combination with Toripalimab and Chemotherapy or RC48-ADC in Combination with Toripalimab and Trastuzumab in First-Line Treatment of Patients with HER2-Expressing Locally Advanced or Metastatic Gastric Cancer (Including Adenocarcinoma of the Gastroesophageal Junction)

The current Example describes the results of a Phase III clinical trial study evaluating the efficacy and safety of RC48-ADC in combination with toripalimab and chemotherapy or RC48-ADC in combination with toripalimab and trastuzumab, the detailed protocol of which is described in Example 6.

Table 4 shows interim safety data for all grades of Common Terminology Criteria for Adverse Events (CTCAE) and treatment related adverse events (TRAE) Grade ≥3. Fewer cases of some Grade ≥3 adverse events, e.g., reduced neutrophils, anemia, reduced platelet count, were observed for treatment 1 and 2 compared to the control group.

TABLE 4
HER2 positive cohort: All grades of CTCAE and TRAE Grade ≥3

| | | | Treatment group 2: | | Control group: | |
|---|---|---|---|---|---|---|
| | Treatment group 1: | | DV + toripalimab + | | toripalimab + | |
| | DV + toripalimab + | | trastuzumab | | trastuzumab + | |
| | CAPOX (n = 18) | | (n = 17) | | CAPOX (n = 17) | |
| | all grades | grade ≥3 | all grades | grade ≥3 | all grades | grade ≥3 |
| | n, (%) | n, (%) | n, (%) | n, (%) | n, (%) | n, (%) |
| Diarrhea | 16 (88%) | 2(11%) | 4 (23%) | 0 | 4 (23%) | 0 |
| Reduced neutrophil count | 12 (66%) | 5(27%) | 11 (64%) | 4 (23%) | 7 (41%) | 0 |
| Reduced white blood cell count | 9 (50%) | 1(5%) | 8 (47%) | 0 | 3 (17%) | 0 |
| Reduced platelet count | 9 (50%) | 1(5%) | 3 (17%) | 0 | 10 (58%) | 33 (17%) |
| Nausea | 12 (66%) | 1(5%) | 3 (17%) | 0 | 10 (58%) | 0 |
| Anemia | 11 (61%) | 0 | 12 (70%) | 0 | 12 (70%) | 22 (11%) |
| Elevated lipase | 6 (33%) | 1 (5%) | 3 (17%) | 0 | 3 (17%) | 0 |
| Elevated amylase | 3 (16%) | 1 (5%) | 3 (17%) | 0 | 3 (17%) | 0 |
| Decreased appetite | 2 (11%) | 0 | 1 (5%) | 0 | 2 (11%) | 0 |
| Peripheral Sensory Neuropathy | 4 (36.3%) | 0 | 2 (11%) | 1 (5%) | 1(5%) | 0 |
| Infusion Reaction | 0 | 0 | 6 (35%) | 1 (5%) | 2 (11%) | 0 |
| Intestinal Obstruction | 0 | 0 | 2 (11%) | 1 (5%) | 1(5%) | 0 |
| Cholecystitis | 0 | 0 | 0 | 1 (5%) | 0 | 0 |

Table 5 shows interim safety all grades of Common Terminology Criteria for Adverse Events (CTCAE) and treatment related adverse events (TRAE) Grade ≥3.

TABLE 5
HER2 low cohort: All grades of CTCAE and TRAE Grade ≥3

| | Treatment group: | | | | Control group: | | | |
|---|---|---|---|---|---|---|---|---|
| | DV + toripalimab + | | | | toripalimab + | | | |
| | CAPOX | | | | CAPOX | | | |
| | (n = 29) | | | | (n = 24) | | | |
| | all grades | | grade ≥3 | | all grades | | grade ≥3 | |
| | n, (%) | | n, (%) | | n, (%) | | n, (%) | |
| Diarrhea | 19 | (65%) | 4 | (13%) | 6 | (25%) | 0 | |
| Reduced | 18 | (62%) | 9 | (31%) | 15 | (62%) | 4 | (16%) |
| neutrophil | | | | | | | | |
| count | | | | | | | | |
| Reduced | 19 | (65%) | 2 | (6%) | 12 | (50%) | 2 | (8%) |
| white blood | | | | | | | | |
| cell count | | | | | | | | |
| Reduced | 14 | (48%) | 11 | (3%) | 15 | (62%) | 3 | (12%) |

| platelet count | | | | | | | |
|---|---|---|---|---|---|---|---|
| Nausea | 18 | (62%) | 0 | | 7 | (29%) | 0 |
| Anemia | 16 | (55%) | 11 | (3%) | 12 | (50%) | 0 |
| Elevated lipase | 8 | (27%) | 0 | | 4 | (16%) | 0 |
| Elevated | 5 | (17%) | 0 | | 5 | (20%) | 0 |
| amylase | | | | | | | |
| Decreased | 8 | (27%) | 0 | | 4 | (16%) | 0 |
| appetite | | | | | | | |
| Peripheral | 4 | (13%) | 0 | | 2 | (8%) | 0 |
| Sensory | | | | | | | |
| Neuropathy | | | | | | | |

| | | | | | |
|---|---|---|---|---|---|
| Infusion | 0 | 0 | 1 | (4%) | 0 |
| Reaction | | | | | |

| | | | | | | |
|---|---|---|---|---|---|---|
| Intestinal | 1 | (3%) | 11 | (3%) | 0 | 0 |

| | | | | |
|---|---|---|---|---|
| Obstruction | | | | |
| Cholecystitis | 0 | 0 | 0 | 0 |

As of Jun. 19, 2024, 95 patients were enrolled, with 48 patients in the HER2-positive cohort and 47 patients in the HER2-low cohort.

Tables 6 and 7 shows the overall response rate (ORR) and disease control rate (DCR) for each treatment group in each of the HER2-low and HER2-positive cohorts. For HER2-positive patients, ORR and DCR are comparably favorable between the two treatment groups compared to the control group, with some improvement over control observed in Treatment 2 (DV+toripalimab+trastuzumab) in terms of ORR. For HER2-low patients, addition of DV to the treatment combination with toripalimab+CAPOX resulted in unexpected improvement in the percentage of ORR.

Overall, disitamab vedotin (RC48) combined with toripalimab and CAPOX showed good safety and excellent efficacy in the treatment of HER2-expressing locally advanced or metastatic gastric cancer, especially for HER2-low locally advanced or metastatic gastric cancer.

TABLE 6
ORR and DCR for HER2-positive cohort, by treatment

| | HER2-POSITIVE | | |
|---|---|---|---|
| | TREATMENT1: | TREATMENT2: | |
| | Disitamab | Disitamab | CONTROL: |
| | vedotin + | vedotin + | Toripalimab + |
| | toripalimab + | toripalimab + | trastuzumab + |
| | CAPOX | trastuzumab | CAPOX |
| | (N = 15) | (N = 17) | (N = 16) |
| ORR (%) | 73.3% (11/15) | 94.1% (16/17) | 81.3% (13/16) |
| DCR (%) | 100% | 100% | 93.8% |

TABLE 7
ORR and DCR for HER2-low cohort, by treatment

HER2-LOW

| | TREATMENT1: | CONTROL: |
|---|---|---|
| | Disitamab vedotin + | Toripalimab + |
| | toripalimab + CAPOX | CAPOX |
| | (N = 24) | (N = 23) |
| ORR (%) | 66.7% (16/24) | 43.5% (10/23) |
| DCR (%) | 100% | 100% |

1. A method for treating or preventing progression of cancer in an individual, comprising administering to the individual an effective amount of an anti-PD-1 antibody and an anti-human epidermal growth factor receptor 2 (HER2) antibody-drug conjugate that comprises an anti-HER2 antibody and a cytotoxic molecule; wherein the anti-HER2 antibody comprises a heavy chain comprising a heavy chain variable (VH) domain and a light chain comprising a light chain variable (VL) domain; wherein
(a) the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence NYLFDH (SEQ ID NO:3), or
(b) the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence ARNYLFDHW (SEQ ID NO:12);
wherein the VL domain comprises a CDR-L1 comprising the amino acid sequence KASQDVGTAVA (SEQ ID NO:4), a CDR-L2 comprising the amino acid sequence WASIRHT (SEQ ID NO:5), and a CDR-L3 comprising the amino acid sequence HQFATYT (SEQ ID NO:6), wherein the cancer is gastric cancer, gastroesophageal junction cancer (GEJC), breast cancer, or endometrial cancer.

2. (canceled)

3. (canceled)

4. The method of claim 1, wherein the heavy chain comprises the amino acid sequence of SEQ ID NO:9 or SEQ ID NO: 10 and the light chain comprises the amino acid sequence of SEQ ID NO: 11.

5. (canceled)

6. The method of claim 1, wherein the cytotoxic molecule comprises a tubulin inhibitor or DNA damaging agent.

7. The method of claim 6, wherein the tubulin inhibitor comprises a dolastatin or derivative thereof, auristatin or derivative thereof, or maytansinoid or derivative thereof.

8. The method of claim 7, wherein the tubulin inhibitor comprises monomethyl auristatin E (MMAE), monomethyl auristatin F (MMAF), or auristatin F (AF).

9. (canceled)

10. (canceled)

11. (canceled)

12. The method of claim 1, wherein the antibody-drug conjugate is represented by formula Ab-(L-U) n, wherein Ab is the anti-HER2 antibody, L is a linker between the cytotoxic molecule and the anti-HER2 antibody, U is the conjugated cytotoxic molecule, and n is an integer from 1 to 8, representing the number of cytotoxic molecules bound to the antibody.

13. (canceled)

14. (canceled)

15. The method of claim 1, wherein the antibody-drug conjugate is disitamab vedotin.

16. The method of claim 1, wherein (a) the anti-PD-1 antibody is toripalimab; (b) wherein the antibody comprises a heavy chain comprising the amino acid sequence of SEQ ID NO:21 and a light chain comprising the amino acid sequence of SEQ ID NO:22; or (c) wherein the antibody comprises a VH domain comprising the amino acid sequence of SEQ ID NO: 19 and a VL domain comprises the amino acid sequence of SEQ ID NO:20.

17. (canceled)

18. (canceled)

19. (canceled)

20. (canceled)

21. (canceled)

22. The method of claim 1, wherein the cancer is gastric cancer or gastroesophageal junction cancer (GEJC).

23. (canceled)

24. (canceled)

25. The method of claim 1, wherein the cancer is breast cancer.

26. (canceled)

27. (canceled)

28. (canceled)

29. (canceled)

30. (canceled)

31. (canceled)

32. (canceled)

33. (canceled)

34. (canceled)

35. (canceled)

36. The method of claim 1, wherein the cancer is a hormone receptor (HR)-negative, HER2-low expressing, unresectable locally advanced or metastatic breast cancer; a standard therapy progressed HR-positive, HER2-low expressing, unresectable locally advanced or metastatic breast cancer; a HR+HER2-low expressing breast cancer wherein the individual had received at least one line of chemotherapy; or a HR-HER2-low expressing breast cancer wherein the individual had received at least one line of chemotherapy.

37. (canceled)

38. The method of claim 1, wherein the antibody-drug conjugate is administered to the individual at a dose of 2.0 mg/kg.

39. (canceled)

40. The method of claim 38, wherein the dose is measured using the bovine serum albumin (BSA)-based Extinction Coefficient (EC) method.

41. (canceled)

42. The method of claim 1, wherein the antibody-drug conjugate is administered to the individual every 2 weeks or every 14 days.

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. The method of claim 1, wherein the administration of the anti-PD-1 antibody and the antibody-drug conjugate to an individual with breast cancer results in progression-free survival (PFS) of 4 months or longer.

56. The method of claim 55, wherein the breast cancer is hormone receptor (HR)+HER2-low expressing breast cancer, and the method comprises intravenously administering disitamab vedotin to the individual at 2.0 mg/kg and intravenously administering toripalimab to the individual at 3.0 mg/kg, every 2 weeks or every 14 days, on the same day of each cycle.

57. The method of claim 22, wherein the method further comprises administering a chemotherapy.

58. (canceled)

59. (canceled)

60. (canceled)

61. (canceled)

62. (canceled)

63. (canceled)

64. (canceled)

65. (canceled)

66. (canceled)

67. (canceled)

68. The method of claim 57, wherein the cancer is a HER2-positive, HER2-low, or HER2-zero locally advanced or metastatic gastric cancer; or a HER2-positive, HER2-low, or HER2-zero locally advanced or metastatic gastroesophageal junction adenocarcinoma.

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. A composition comprising an anti-HER2 antibody-drug conjugate for use in a method of treating or preventing progression of cancer in an individual, wherein the method comprises administering an effective amount of an anti-PD-1 antibody and the anti-HER2 antibody-drug conjugate according to the method of any one of claims 1-56 and 88.

90. (canceled)

91. (canceled)

92. (canceled)

93. (canceled)

94. (canceled)

95. (canceled)

96. (canceled)

97. (canceled)

98. (canceled)

99. A kit comprising an anti-PD-1 antibody and an anti-HER2 antibody-drug conjugate that comprises an anti-HER2 antibody and a cytotoxic molecule; wherein the anti-HER2 antibody comprises a heavy chain comprising a heavy chain variable (VH) domain and a light chain comprising a light chain variable (VL) domain; wherein
(i) the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence NYLFDH (SEQ ID NO:3), or
(ii) the VH domain comprises a CDR-H1 comprising the amino acid sequence DYYIH (SEQ ID NO:1), a CDR-H2 comprising the amino acid sequence RVNPDHGDSYYNQKFKD (SEQ ID NO:2), and a CDR-H3 comprising the amino acid sequence ARNYLFDHW (SEQ ID NO:12);
wherein the VL domain comprises a CDR-L1 comprising the amino acid sequence KASQDVGTAVA (SEQ ID NO:4), a CDR-L2 comprising the amino acid sequence WASIRHT (SEQ ID NO:5), and a CDR-L3 comprising the amino acid sequence HQFATYT (SEQ ID NO:6).

100. (canceled)

101. (canceled)

102. (canceled)

103. (canceled)

104. (canceled)

105. (canceled)

106. (canceled)

107. (canceled)

108. (canceled)

109. (canceled)


SEQUENCE LISTING (22 sequences)

SEQ ID NO: 1 - protein, 5 aa, synthetic construct
DYYIH

SEQ ID NO: 2 - protein, 17 aa, synthetic construct
RVNPDHGDSYYNQKFKD

SEQ ID NO: 3 - protein, 6 aa, synthetic construct
NYLFDH

SEQ ID NO: 4 - protein, 11 aa, synthetic construct
KASQDVGTAVA

SEQ ID NO: 5 - protein, 7 aa, synthetic construct
WASIRHT

SEQ ID NO: 6 - protein, 7 aa, synthetic construct
HQFATYT

SEQ ID NO: 7 - protein, 115 aa, synthetic construct
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSS

SEQ ID NO: 8 - protein, 105 aa, synthetic construct
DIQMTQSPSSVSASVGDRVTITCKASQDVGTAVAWYQQKPGKAPKLLIYWASIRHTGVPSRFSGSGSGTDFTLTISSLQPEDFATYYCHQFATYTFGGGTKVEIK

SEQ ID NO: 9 - protein, 444 aa, synthetic construct
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 445 aa, synthetic construct
EVQLVQSGAEVKKPGATVKISCKVSGYTFTDYYIHWVQQAPGKGLEWMGRVNPDHGDSYYNQKFKDKATITADKSTDTAYMELSSLRSEDTAVYFCARNYLFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 212 aa, synthetic construct
DIQMTQSPSSVSASVGDRVTITCKASQDVGTAVAWYQQKPGKAPKLLIYWASIRHTGVPSRFSGSGSGTDFTLTISSLQPEDFATYYCHQFATYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 9 aa, synthetic construct
ARNYLFDHW

SEQ ID NO: 13 - protein, 5 aa, synthetic construct
DYEMH

SEQ ID NO: 14 - protein, 17 aa, synthetic construct
VIESETGGTAYNQKFKG

SEQ ID NO: 15 - protein, 16 aa, synthetic construct
EGITTVATTYYWYFDV

SEQ ID NO: 16 - protein, 16 aa, synthetic construct
RSSQSIVHSNGNTYLE

SEQ ID NO: 17 - protein, 7 aa, synthetic construct
KVSNRFS

SEQ ID NO: 18 - protein, 9 aa, synthetic construct
FQGSHVPLT

SEQ ID NO: 19 - protein, 125 aa, synthetic construct [sequence omitted]

SEQ ID NO: 20 - protein, 115 aa, synthetic construct
DVVMTQSPLSLPVTLGQPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIKRTV

SEQ ID NO: 21 - protein, 452 aa, synthetic construct
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 219 aa, synthetic construct
DVVMTQSPLSLPVTLGQPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC